(19) United States
(12) Patent Application Publication
(10) Pub. No.: US 2024/0019378 A1
(43) Pub. Date: Jan. 18, 2024

(54) SYSTEMS AND METHODS FOR DETECTING FOODBORNE PATHOGENS BY ANALYZING SPECTRAL DATA

(71) Applicant: Hyperspectral Corp., Alexandria, VA (US)

(72) Inventors: Matthew Theurer, Alexandria, VA (US); Sarah Rachel Hernandez, Austin, TX (US)

(73) Assignee: Hyperspectral Corp., Alexandria, VA (US)

(21) Appl. No.: 18/346,749

(22) Filed: Jul. 3, 2023

Related U.S. Application Data
(60) Provisional application No. 63/367,717, filed on Jul. 5, 2022.

(51) Int. Cl. G01N 21/78; G01N 21/31; G01N 33/02

(52) U.S. Cl. CPC G01N 21/78; G01N 21/31; G01N 33/02; G01N 2201/129

(57) ABSTRACT

An example method includes receiving a first set of values based on a set of intensity measurements. The set of intensity measurements may be obtained by a light intensity measuring apparatus that measured intensities of light that passed through a sample of a food processing byproduct. A second set of values based on the first set of values may be generated. A set of trained decision trees may be applied to the second set of values to obtain a result. Based on the result, either a positive foodborne pathogen detection or a negative foodborne pathogen detection for a foodborne pathogen in the sample of the food processing byproduct may be determined. A foodborne pathogen detection notification that indicates either the positive foodborne pathogen detection or the negative foodborne pathogen detection for the foodborne pathogen in the sample of the food processing byproduct may be generated and provided.

### CROSS-REFERENCE TO RELATED APPLICATIONS

This application claims priority to U.S. Provisional Patent Application No. 63/367,717, filed on Jul. 5, 2022, and entitled “SYSTEMS AND METHODS FOR USING A MULTI-MODEL ML SYSTEM WITH HYPERSPECTRAL DATA USING TIME-BASED WAVELENGTH SPECTRAL DATA”, and is related to co-pending application U.S. patent application Ser. No. 18/173,050, filed on Feb. 22, 2023 and entitled “SYSTEMS AND METHODS FOR DETECTING PATHOGENS USING SPECTROMETER SCANS” and co-pending application U.S. patent application Ser. No. 18/173,035, filed on Feb. 22, 2023 and entitled “SYSTEMS AND METHODS FOR DETECTING FOODBORNE PATHOGENS USING SPECTRAL ANALYSIS”, each of which is incorporated in its entirety herein by reference.

### FIELD OF THE INVENTION(S)

Embodiments of the present invention(s) are generally related to detecting foodborne pathogens by analyzing spectral data, and in particular to detecting foodborne pathogens by analyzing spectral data from spectrometer scans of food processing byproducts.

### BACKGROUND

Foodborne illnesses may be caused by consuming food or beverages that are contaminated by pathogens such as bacteria, toxins produced by bacteria, viruses, parasites, chemicals, foreign material (e.g., metal shavings) and/or the like. The United States Food and Drug Administration (U.S. FDA) estimates that there are approximately 48 million cases of foodborne illness each year in the United States. The U.S. FDA further estimates that 1 in 6 Americans are affected by foodborne illnesses, resulting in 128,000 hospitalizations and 3,000 deaths.

Food or beverages (collectively, food) may be contaminated during any stage in the supply chain (e.g., in the field, while undergoing processing at food production or processing facilities (collectively, food processing facilities), or during shipping or transport). However, the contamination may not be discovered until after people are sickened from consuming the food. Unfortunately, government agencies, such as the U.S. FDA, often declare an outbreak of a foodborne illness and issue recalls of the food suspected of causing the outbreak only after a number of people are sickened.

In addition to the deleterious effects on individual health, there are economic costs to recalls. For example, a food producer or processor (collectively, a food processor) may voluntarily or be required to recall numerous lots of food or entire production runs. Such recalls may sicken many and may tarnish the brand of the food processor, leading to consumer distrust reduced sales, and large costs for product recalls, legal defense, and damage control.

### SUMMARY

In some aspects, the techniques described herein relate to a non-transitory computer-readable medium including executable instructions, the executable instructions being executable by one or more processors to perform a method, the method including: receiving a first set of values, the first set of values based on a set of intensity measurements for a set of wavelengths of light, the set of intensity measurements for the set of wavelengths of light obtained by an apparatus configured to generate light, detect the light that has passed through at least a portion of a sample of a food processing byproduct, and measure intensities of the light to obtain the set of intensity measurements for the set of wavelengths of light; generating a second set of values based on the first set of values; applying a set of trained decision trees to the second set of values to obtain a result, the set of trained decision trees trained on a set of training samples, a first subset of training samples of the set of training samples containing a foodborne pathogen at a first concentration and a second subset of training samples of the set of training samples containing the foodborne pathogen at a second concentration different from the first concentration; based on the result, determining either a positive foodborne pathogen detection or a negative foodborne pathogen detection for the foodborne pathogen in the sample of the food processing byproduct; generating a foodborne pathogen detection notification that indicates either the positive foodborne pathogen detection or the negative foodborne pathogen detection for the foodborne pathogen in the sample of the food processing byproduct; and providing the foodborne pathogen detection notification.

In some aspects, the techniques described herein relate to a non-transitory computer-readable medium, the method further including based on the result, determining an approximate concentration or an approximate range of concentrations for the foodborne pathogen in the sample of the food processing byproduct, and wherein the foodborne pathogen detection notification further indicates the approximate concentration or the approximate range of concentrations for the foodborne pathogen in the sample of the food processing byproduct.

In some aspects, the techniques described herein relate to a non-transitory computer-readable medium wherein applying the set of trained decision trees to the second set of values to obtain the result further obtains a confidence value for the foodborne pathogen in the sample of the food processing byproduct, and wherein the foodborne pathogen detection notification further indicates the confidence value for the foodborne pathogen in the sample of the food processing byproduct.

In some aspects, the techniques described herein relate to a non-transitory computer-readable medium wherein the set of trained decision trees is a first set of trained decision trees, the set of training samples is a first set of training samples, the foodborne pathogen is a first foodborne pathogen, the result is a first result, the positive foodborne pathogen detection is a first positive foodborne pathogen detection, the negative foodborne pathogen detection is a first negative foodborne pathogen detection, and the foodborne pathogen detection notification is a first foodborne pathogen detection notification, the method further including: applying a second set of trained decision trees to the second set of values to obtain a second result, the second set of trained decision trees trained on a second set of training samples, a third subset of training samples of the second set of training samples containing a second foodborne pathogen at a third concentration and a fourth subset of training samples of the second set of training samples containing the second foodborne pathogen at a fourth concentration different from the third concentration, the second foodborne pathogen different from the first foodborne pathogen; based on the second result, determining either a second positive foodborne pathogen detection or a second negative foodborne pathogen detection for the second foodborne pathogen in the sample of the food processing byproduct; generating a second foodborne pathogen detection notification indicating either the second positive foodborne pathogen detection or the second negative foodborne pathogen detection for the second foodborne pathogen in the sample of the food processing byproduct; and providing the second foodborne pathogen detection notification.

In some aspects, the techniques described herein relate to a non-transitory computer-readable medium wherein the set of intensity measurements for the set of wavelengths of light is a first set of intensity measurements for the set of wavelengths of light, the result is a first result, the method further including: receiving at least one third set of values, the at least one third set of values based on at least one second set of intensity measurements for the set of wavelengths of light, the at least one second set of intensity measurements for the set of wavelengths of light obtained by the apparatus; generating at least one fourth set of values based on the at least one third set of values; and applying the set of trained decision trees to the at least one fourth set of values to obtain at least one second result, wherein based on the first result, determining either the positive foodborne pathogen detection or the negative foodborne pathogen detection for the foodborne pathogen in the sample of the food processing byproduct includes based on the first result and the at least one second result, determining either the positive foodborne pathogen detection or the negative foodborne pathogen detection for the foodborne pathogen in the sample of the food processing byproduct.

In some aspects, the techniques described herein relate to a non-transitory computer-readable medium wherein generating the second set of values based on the first set of values includes normalizing each value in the second set of values to be between zero, inclusive, and one, inclusive.

In some aspects, the techniques described herein relate to a non-transitory computer-readable medium, the method further including training a set of decision trees on the set of training samples to obtain the set of trained decision trees.

In some aspects, the techniques described herein relate to a non-transitory computer-readable medium wherein at least some training samples of the set of training samples correspond to a particular food processing facility, a region that includes multiple food processing facilities, or one or more classes of food processing facilities.

In some aspects, the techniques described herein relate to a non-transitory computer-readable medium wherein values in the first set of values are one of absorbance values and transmittance values.

In some aspects, the techniques described herein relate to a non-transitory computer-readable medium wherein the sample of the food processing byproduct is mixed with a reagent.

In some aspects, the techniques described herein relate to a non-transitory computer-readable medium wherein the result indicates the positive foodborne pathogen detection if the result meets or exceeds a threshold.

In some aspects, the techniques described herein relate to a non-transitory computer-readable medium wherein the set of wavelengths of light includes wavelengths of light in ultraviolet, visible, and infrared spectrums.

In some aspects, the techniques described herein relate to a non-transitory computer-readable medium wherein the set of wavelengths of light includes wavelengths of light ranging from approximately 300 nanometers to approximately 1100 nanometers.

In some aspects, the techniques described herein relate to a system including at least one processor and memory containing executable instructions, the executable instructions being executable by the at least one processor to: receive a first set of values, the first set of values based on a set of intensity measurements for a set of wavelengths of light, the set of intensity measurements for the set of wavelengths of light obtained by an apparatus configured to generate light, detect the light that has passed through at least a portion of a sample of a food processing byproduct, and measure intensities of the light to obtain the set of intensity measurements for the set of wavelengths of light; generate a second set of values based on the first set of values; apply a set of trained decision trees to the second set of values to obtain a result, the set of trained decision trees trained on a set of training samples, a first subset of training samples containing a foodborne pathogen at a first concentration and a second subset of training samples containing the foodborne pathogen at a second concentration different from the first concentration; based on the result, determine either a positive foodborne pathogen detection or a negative foodborne pathogen detection for the foodborne pathogen in the sample of the food processing byproduct; generate a foodborne pathogen detection notification that indicates either the positive foodborne pathogen detection or the negative foodborne pathogen detection for the foodborne pathogen in the sample of the food processing byproduct; and provide the foodborne pathogen detection notification.

In some aspects, the techniques described herein relate to a system, the executable instructions being further executable by the at least one processor to based on the result, determine an approximate concentration or an approximate range of concentrations for the foodborne pathogen in the sample of the food processing byproduct, and wherein the foodborne pathogen detection notification further indicates the approximate concentration or the approximate range of concentrations for the foodborne pathogen in the sample of the food processing byproduct.

In some aspects, the techniques described herein relate to a system wherein the executable instructions to apply the set of trained decision trees to the second set of values to obtain the result include executable instructions to obtains a confidence value for the foodborne pathogen in the sample of the food processing byproduct, and wherein the foodborne pathogen detection notification further indicates the confidence value for the foodborne pathogen in the sample of the food processing byproduct.

In some aspects, the techniques described herein relate to a system wherein the set of trained decision trees is a first set of trained decision trees, the set of training samples is a first set of training samples, the foodborne pathogen is a first foodborne pathogen, the result is a first result, the positive foodborne pathogen detection is a first positive foodborne pathogen detection, the negative foodborne pathogen detection is a first negative foodborne pathogen detection, and the foodborne pathogen detection notification is a first foodborne pathogen detection notification, and the executable instructions being further executable by the at least one processor to: apply a second set of trained decision trees to the second set of values to obtain a second result, the second set of trained decision trees trained on a second set of training samples, a third subset of training samples containing a second foodborne pathogen at a third concentration and a fourth subset of training samples containing the second foodborne pathogen at a fourth concentration different from the third concentration, the second foodborne pathogen different from the first foodborne pathogen; based on the second result, determine either a second positive foodborne pathogen detection or a second negative foodborne pathogen detection for the second foodborne pathogen in the sample of the food processing byproduct; generate a second foodborne pathogen detection notification indicating either the second positive foodborne pathogen detection or the second negative foodborne pathogen detection for the second foodborne pathogen in the sample of the food processing byproduct; and provide the second foodborne pathogen detection notification.

In some aspects, the techniques described herein relate to a system wherein the executable instructions being executable by the at least one processor to generate the second set of values based on the first set of values include executable instructions being executable by the at least one processor to normalize each value in the second set of values to be between zero, inclusive, and one, inclusive.

In some aspects, the techniques described herein relate to a system, the executable instructions being further executable by the at least one processor to train a set of decision trees on the set of training samples to obtain the set of trained decision trees.

In some aspects, the techniques described herein relate to a system wherein at least some training samples of the set of training samples correspond to a particular food processing facility, a region that includes multiple food processing facilities, or one or more classes of food processing facilities.

In some aspects, the techniques described herein relate to a system wherein values in the first set of values are one of absorbance values and transmittance values.

In some aspects, the techniques described herein relate to a system wherein the sample of the food processing byproduct is mixed with a reagent.

In some aspects, the techniques described herein relate to a system wherein the result indicates the positive foodborne pathogen detection if the result meets or exceeds a threshold.

In some aspects, the techniques described herein relate to a system wherein the set of wavelengths of light includes wavelengths of light in ultraviolet, visible, and infrared spectrums.

In some aspects, the techniques described herein relate to a method including: receiving a first set of values, the first set of values based on a set of intensity measurements for a set of wavelengths of light, the set of intensity measurements for the set of wavelengths of light obtained by an apparatus configured to generate light, detect the light that has passed through at least a portion of a sample of a food processing byproduct, and measure intensities of the light to obtain the set of intensity measurements for the set of wavelengths of light; generating a second set of values based on the first set of values; applying a set of trained decision trees to the second set of values to obtain a result, the set of trained decision trees trained on a set of training samples, a first subset of training samples containing a foodborne pathogen at a first concentration and a second subset of training samples containing the foodborne pathogen at a second concentration different from the first concentration; based on the result, determining either a positive foodborne pathogen detection or a negative foodborne pathogen detection for the foodborne pathogen in the sample of the food processing byproduct; generating a foodborne pathogen detection notification indicating either the positive foodborne pathogen detection or the negative foodborne pathogen detection for the foodborne pathogen in the sample of the food processing byproduct; and providing the foodborne pathogen detection notification.

### BRIEF DESCRIPTION OF THE DRAWINGS

FIG. 1 depicts an example foodborne pathogen detection environment in some embodiments.

FIG. 2A depicts an example food processing apparatus, an example computing device, and an example light intensity measuring apparatus in some embodiments.

FIG. 2B depicts another example food processing apparatus, an example computing device, and an example light intensity measuring apparatus in some embodiments.

FIG. 3A is a block diagram of components of an example computing device in some embodiments.

FIG. 3B is a block diagram of components of a foodborne pathogen detection system in some embodiments.

FIG. 4 is a flowchart showing a method for detecting foodborne pathogens in some embodiments.

FIG. 5 is a flowchart showing a method for training sets of decision trees for detecting foodborne pathogens in some embodiments.

FIG. 6 depicts a graph of absorption for a set of wavelengths for multiple instances of light that passed through multiple samples containing different concentrations of E. coli and multiple samples that do not contain E. coli in some embodiments.

FIG. 7A depicts a confusion matrix for results of applications of a set of trained decision trees to multiple sets of values based on intensity measurements of multiple instances of light that has passed through multiple testing samples containing E. coli at seven different concentrations and multiple testing samples that do not contain E. coli in some embodiments.

FIG. 7B depicts a confusion matrix for results of applications of a set of trained decision trees to multiple sets of values based on intensity measurements of multiple instances of light that has passed through multiple testing samples containing E. coli at four different concentrations and multiple testing samples that do not contain E. coli in some embodiments.

FIG. 8 depicts a confusion matrix for results of applications of a set of trained decision trees to multiple sets of values based on intensity measurements of multiple instances of light that has passed through multiple testing samples containing E. coli at seven different concentrations and multiple testing samples that do not contain E. coli in some embodiments.

FIG. 9 depicts a graph of absorption for a set of wavelengths for multiple instances of light that passed through multiple samples containing different concentrations of microspheres in some embodiments.

FIGS. 10A and 10B depict confusion matrices for results of applications of two sets of trained decision trees to multiple sets of values based on intensity measurements of multiple instances of light that has passed through samples containing 1000 nanometer microspheres or samples that do not contain microspheres in some embodiments.

FIG. 11 depicts a confusion matrix for results of applications of a set of trained decision trees to multiple sets of values based on intensity measurements of multiple instances of light that has passed through samples containing 500 nanometer microspheres or samples that do not contain microspheres in some embodiments.

FIGS. 12A and 12B depict confusion matrices for results of applications of two sets of trained decision trees to multiple sets of values based on intensity measurements of multiple instances of light that has passed through samples containing 50 nanometer microspheres or samples that do not contain microspheres in some embodiments.

FIG. 13 depicts a confusion matrix for results of applications of a set of trained decision trees to multiple sets of values based on intensity measurements of multiple instances of light that has passed through samples containing green microspheres at ten different concentrations or samples that do not contain microspheres in some embodiments.

FIG. 14A depicts a confusion matrix for results of applications of a set of trained decision trees to multiple sets of values based on intensity measurements of multiple instances of light that has passed through samples containing green microspheres, samples containing red microspheres, or samples that do not contain microspheres in some embodiments.

FIG. 14B depicts a confusion matrix for results of applications of a set of trained decision trees to multiple sets of values based on intensity measurements of multiple instances of light that has passed through samples containing green microspheres, samples containing red microspheres, samples containing a mixture of red microspheres and green microspheres, or samples that do not contain microspheres in some embodiments.

FIG. 15 depicts an example vortex spectrometer in some embodiments.

FIG. 16A depicts an example coronagraph scheme including a concentric circular surface relief grating with rectangular grooves with depth h and a periodicity of A.

FIG. 16B includes images of amplitude and phase caused by the vortex mask in some embodiments.

FIG. 16C depicts an example of a vortex mask which can be seen as a polarization FQ-PM.

FIG. 17A depicts an example simplified spectrometer optical path in some embodiments.

FIG. 17B depicts another example simplified spectrometer optical path in some embodiments.

FIG. 17C is another example of an optical path of a spectrometer in some embodiments.

FIG. 18A depicts a measurement of the aperture of an entrance aperture as being 6 mm in one example.

FIG. 18B depicts a measurement of an optical beam received and reflected by a deformable mirror in some embodiments.

FIG. 19 depicts the irradiance at the entrance to the vortex mask as 34 micrometers in one example.

FIG. 20A depicts a field modulus (amplitude) after the vortex mask in some embodiments.

FIG. 20B depicts a field phase (radians) after the vortex mask in some embodiments.

FIG. 21A depicts an example interior irradiance of a lyot stop in one example.

FIG. 21B is a graph indicating a 10-3 contrast for a lyot stop radius of 1.25 mm in one example.

FIG. 22 is a flowchart for identifying pathogens from spectrometer data in some embodiments.

FIG. 23A depicts a test spectra in one example.

FIG. 23B depicts a reference spectra in one example.

FIG. 23C depicts the mean value of the dark noise in one example.

FIG. 23D depicts a test spectra of dark noise corrected in one example.

FIG. 23E depicts a reference spectra of dark noise corrected in one example.

FIG. 24A depicts an example test spectra including spectra normalization averaged over instances.

FIG. 24B depicts an example reference spectra including spectra normalization averaged over instances.

FIG. 24C depicts a test spectra with spectra normalization for the first sample, all instances.

FIG. 24D depicts an example reference spectra including spectra normalization for the first sample, all instances.

FIG. 25A depicts an example test spectra including spectra normalization averaged over instances.

FIG. 25B depicts an example reference spectra including spectra normalization averaged over instances.

FIG. 26A depicts an example test spectra of positive (infection) results with background suppression.

FIG. 26B depicts an example test spectra of negative (infection) results with background suppression.

FIG. 26C depicts an example test spectra of positive (infection) results with background suppression.

FIG. 26D depicts an example test spectra of negative (infection) results with background suppression.

FIG. 27A depicts a negative result scalogram conversion after wavelet correlation.

FIG. 27B depicts a positive result scalogram conversion after wavelet correlation.

FIG. 27C depicts a difference between the positive and negative result scalogram conversion depicting the difference and indicating the signature of infection.

FIG. 28 depicts examples of lucky imaging in some embodiments.

FIG. 29 depicts a system for spectral reconstruction based on scattered light caused by a diversifier in some embodiments.

FIG. 30 depicts a system for spectral reconstruction based on scattered light caused by a diversifier but without an external light source in some embodiments.

FIG. 31 is an example of a speckle pattern in some embodiments.

FIG. 32 depicts an example optical vortex meta-surface.

FIG. 33 depicts an optical vortex plasmonic matrix that may be utilized rather than the diffuser, speckle pattern, or optical vortex meta-surface.

FIG. 34 is a flowchart for creating filtering for spectral reconstruction in some embodiments.

FIG. 35 depicts an example architecture of the neural network.

FIG. 36 is an example spectra taken from the data collected from the spectrometer.

FIG. 37 is a spectra that was reconstructed by the neural network.

FIG. 38 depicts an example mapping of an optical vortex plasmonic array to a non-integer topological charge lₘₙ.

FIG. 39 is an example graphic of light shining on a non-integer vortex array waveplate and an example resulting image in some embodiments.

FIG. 40 depicts an example deep neural network and example configuration in some embodiments.

FIG. 41 includes comparisons of actual spectra plotted against reconstructed spectra in testing in some embodiments.

FIG. 42 depicts a block diagram of an example digital device in some embodiments.

Throughout the drawings, like reference numerals will be understood to refer to like parts, components, and structures.

### DETAILED DESCRIPTION

A government agency such as the U.S. FDA may not declare a foodborne illness outbreak until after a large number of persons have been sickened. Before declaring the outbreak, the government agency may have to perform an investigation to determine the food that is causing the outbreak, which may be difficult to do and/or take significant time. If the government agency is able to determine the food, testing for foodborne pathogens has to be performed to identify the particular foodborne pathogen responsible for the foodborne illnesses. The investigation and testing may take a large amount of time, during which more persons may be affected by the contaminated food. One reason for the large amount of time is that it may take approximately 48 hours to approximately 72 hours to obtain test results confirming a foodborne pathogen.

In various embodiments, systems and methods discussed herein may enable early detection of foodborne pathogens during food production or processing (collectively, food processing) at food processing facilities. The systems may utilize light intensity measuring apparatuses, which may be or include spectrometers or spectrophotometers, to scan water used or produced by food processing apparatuses. The light intensity measuring apparatuses may transmit the spectrometer scans to a foodborne pathogen detection system that utilizes machine learning (ML) and/or artificial intelligence (AI) models to detect evidence of foodborne pathogens in the spectrometer scans. The foodborne pathogen detection system may provide results to personnel working in the food processing facilities. In the event of a positive detection of a foodborne pathogen, the personnel may stop food processing and start remedial measures, such as cleaning food processing equipment, discarding contaminated food, and/or performing additional testing or detection.

Such early detection of foodborne pathogens allows food processors to identify contaminated food prior to shipping the food out to wholesalers, distributors, retailers, and/or consumers. This early detection may save food processors the costs of recalling food, which may be significant. In addition, early detection may prevent or reduce the occurrence of foodborne illness outbreaks, which may prevent or reduce illnesses, hospitalizations, and deaths.

In various embodiments, the systems and methods described herein are applicable to detect a wide variety of foodborne pathogens that cause foodborne illnesses. Such foodborne pathogens include norovirus, salmonella (non-typhoidal), Clostridium perfringens, campylobacter, Staphylococcus aureus, Toxoplasma gondii, Escherichia coli (E. coli), Clostridium botulinum, cryptosporidium, Cyclospora, hepatitis A virus, shigella, Yersinia, and Listeria monocytogenes (listeria), among many others. The foodborne pathogen detection systems may train one or more ML and/or AI models for each foodborne pathogen. Upon receiving spectral data from light intensity measuring apparatuses, the foodborne pathogen detection systems may apply the trained machine learning and/or AI models to the spectral data. In this way, the foodborne pathogen detection systems may be able to detect multiple foodborne pathogens from spectral data of a single sample of a food processing byproduct. One advantage of some embodiments of the systems and methods described herein is that they may decrease the Limit of Detection (LOD) from the Classical Limit of Detection (cLOD), that is limited by physics, to the machine learning limit of detection (mlLOD) that may be one to two orders of magnitude lower than the cLOD.

In various embodiments, the light intensity measuring apparatuses may be or include spectrometers or other spectral analysis technology, such as commercially available spectrometers or customized UV/VIS/NIR/MWIR/LWIR sensors that are capable of communicating with the foodborne pathogen detection system or are couplable to digital devices capable of communicating with the foodborne pathogen detection system. Food processors may widely deploy the light intensity measuring apparatuses at food processing facilities to detect foodborne pathogens in their food processing. The foodborne pathogen detection systems and associated methods described herein, because they provide more accurate results more quickly and economically than other systems and methods, are broadly applicable to any location where food is processed, such as farms, food processing facilities, packaging facilities, distributors, restaurants, grocery stores, homes, and other locations. Accordingly, the foodborne pathogen detection systems and associated methods described herein may provide significant benefits to farmers, food processors, distributors, restaurant operators, grocery store operators, households, consumers, and others (e.g., any entity in the farm to fork supply chain).

The foodborne pathogen detection systems and associated methods, due to the ability to perform rapid and continuous testing of foods, also allow for food processors to quarantine food that may be contaminated by foodborne pathogens prior to shipping out such food. For example, a food processor, upon detection of a foodborne pathogen during a particular food processing run, may be able to quarantine food processed during that run or food processed after the last “clean” test prior to shipping out that food. The food processor may then test the food (e.g., using laboratory tests) to confirm the presence of foodborne pathogens. The food processor may also be able to clean food processing equipment and/or parts of the food processing facility to prevent or reduce contamination of further food. The food processor may then retest food processing byproducts and/or equipment for contamination. As a result, the food processor may confirm that the machinery and/or byproducts are “clean” (e.g., without detected foodborne pathogens) before returning to food processing.

Accordingly, food processors may be able to reduce economic costs associated with foodborne illness outbreaks. Furthermore, effects on individual health and/or public health may be avoided or reduced by the deployment of the foodborne pathogen detection systems and associated methods described herein.

The foodborne pathogen detection systems and associated methods described herein may also aid food processors in complying with food safety laws and regulations, such as those promulgated by government agencies such as the U.S. FDA.

The foodborne pathogen detection systems and associated methods may also be utilized to detect pathogens and/or contaminants that may affect water quality. Accordingly, the foodborne pathogen detection systems and associated methods described herein may also aid community water systems and/or other water suppliers with complying with water quality standards, such as those promulgated by government agencies such as the U.S. Environmental Protection Agency.

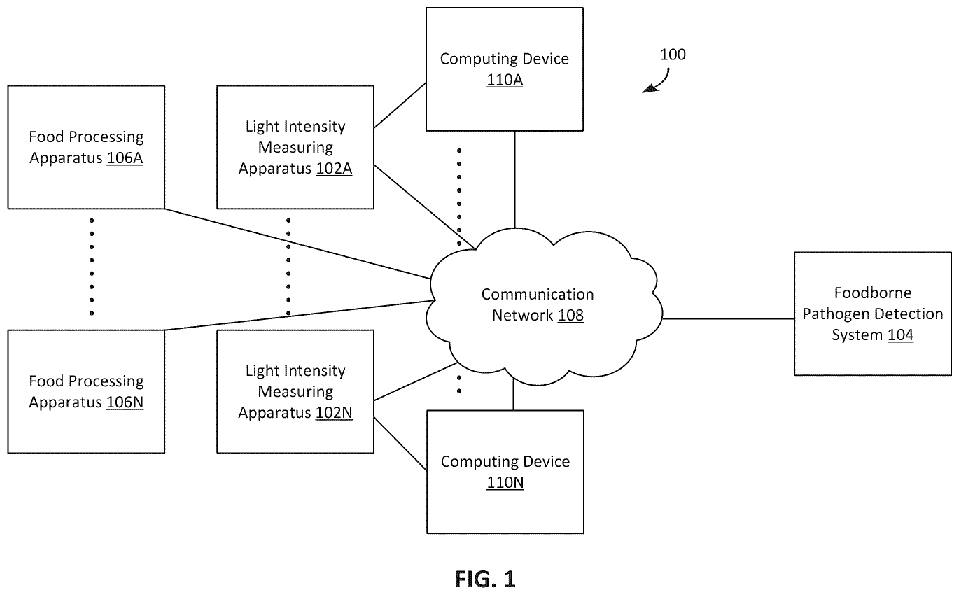

FIG. 1 depicts an example foodborne pathogen detection environment 100 in some environments. The foodborne pathogen detection environment 100 includes food processing apparatuses 106A to 106N (referred to herein as a food processing apparatus 106 or as food processing apparatuses 106), light intensity measuring apparatuses 102A to 102N (referred to herein as a light intensity measuring apparatus 102 or as light intensity measuring apparatuses 102), computing devices 110A to 110N (referred to herein as a computing device 110 or as computing devices 110) communication network 108, and a foodborne pathogen detection system 104. Although a single foodborne pathogen detection system 104 is depicted in FIG. 1, the foodborne pathogen detection environment 100 may include any number of foodborne pathogen detection systems 104. The foodborne pathogen detection environment 100 may also include other systems, apparatuses, devices, machines, and/or components not illustrated in FIG. 1, such as cleaning systems, water supply and water drain systems, and/or electrical and communication systems.

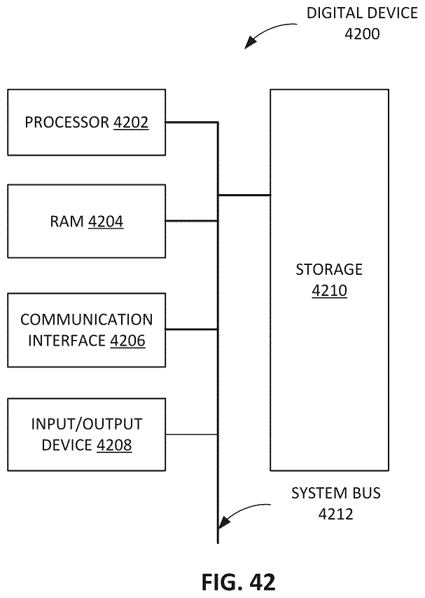

The food processing apparatus 106 may be or include any device, machine and/or apparatus that processes food or facilitates processing food for human or animal consumption. For example, the food processing apparatus 106 may be a washing machine that washes fruits and vegetables such as leafy greens, apples, carrots, and the like using water. As another example, the food processing apparatus 106 may be a commercial spinner that dries washed lettuce and other vegetables, which produces water to be drained away. Other examples of food processing apparatuses 106 are within the scope of this disclosure. The food processing apparatus 106 may be or include any number of digital devices. Digital devices are discussed, for example, with reference to FIG. 42. The 106// may be connected to the 108//.

The light intensity measuring apparatus 102 may be or include any number of digital devices. In one example, the light intensity measuring apparatus 102 may be a Hach DR3900 spectrophotometer of the Hach Company of Loveland, Colorado, United States of America. In another example, the light intensity measuring apparatus 102 may each be or include a different spectrophotometer, spectrometer, sensor, or detector capable of network communication. The light intensity measuring apparatus 102 may perform the functions of a spectrophotometer or a spectrometer, such as the spectrophotometers and spectrometers discussed herein. The light intensity measuring apparatus 102 may receive samples of food processing byproducts, detect light that has passed through the samples, and measure intensities of a set of wavelengths of the light that has passed through the samples. The light intensity measuring apparatus 102 may then transmit a set of values based on the measured intensities for the set of wavelengths of the light to the computing device 110. The set of values may be the measured intensities, or they may be other values based on the measured intensities, such as absorbance or transmittance values.

The computing device 110 may be or include any number of digital devices. In one example, the computing device 110 may be a laptop and may be connected to the light intensity measuring apparatus 102 via a physical cable, such as a Universal Serial Bus (USB) cable. In some embodiments, the light intensity measuring apparatus 102 and the computing device 110 are not directly connected via a physical cable but are indirectly connected through a network, such as an IP-based Local Area Network (LAN), which may be part of or connected to the communication network 108. The computing device 110 may receive the set of values from the light intensity measuring apparatus 102. The computing device 110 may then transmit the set of values to the foodborne pathogen detection system 104.

The foodborne pathogen detection system 104 may be or include any number of digital devices. The foodborne pathogen detection system 104 may receive the set of values, process the set of values as described herein, generate a foodborne pathogen detection notification, and provide the foodborne pathogen detection notification. In some embodiments, the foodborne pathogen detection system 104 provides the foodborne pathogen detection notification to the computing device 110.

The light intensity measuring apparatus 102, the computing device 110, and/or the foodborne pathogen detection system 104 may, in the event of a positive foodborne pathogen detection notification, notify third party systems such as those operated by food processors, those operated by government agencies such as the U.S. FDA, and/or those operated by third parties approved by such government agencies. In such an event, the light intensity measuring apparatus 102, the computing device 110, and/or the foodborne pathogen detection system 104 may also recommend further diagnostic analysis by government agencies or other third parties approved by the government agencies.

In some embodiments, communication network 108 represents one or more computer networks (for example, LANs, WANs, and/or the like). The communication network 108 may provide communication between any of the food processing apparatuses 106, any of the light intensity measuring apparatuses 102, any of the computing devices 110, and the foodborne pathogen detection system 104. In some implementations, the communication network 108 comprises computer devices, routers, cables, and/or other network topologies. In some embodiments, the communication network 108 may be wired and/or wireless. In various embodiments, the communication network 108 may comprise the Internet, one or more networks that may be public, private, IP-based, non-IP based, and so forth.

Some embodiments described herein discuss performing spectral analysis on water samples (e.g., obtained from wash water), such as those obtained directly or indirectly from food processing apparatuses 106. It will be appreciated that the light intensity measuring apparatus 102, the computing device 110, and/or the foodborne pathogen detection system 104 may perform spectral analysis on any food processing byproduct. Examples of food processing byproducts include, but are not limited to, water, wash water, oils, greases, animal blood, meat, and feces from animals such as cows, pigs, chickens. Furthermore, samples may be obtained by swabbing or otherwise sampling food processing equipment, surfaces, residues, or anything that comes into contact with food. It will be understood that food processing byproducts are not limited to the examples described herein.

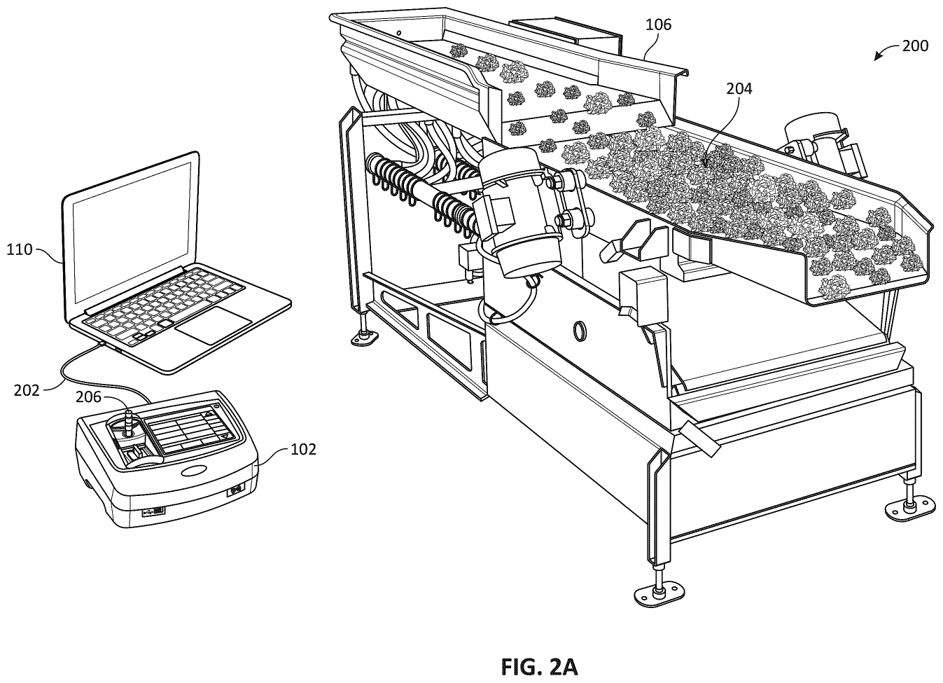

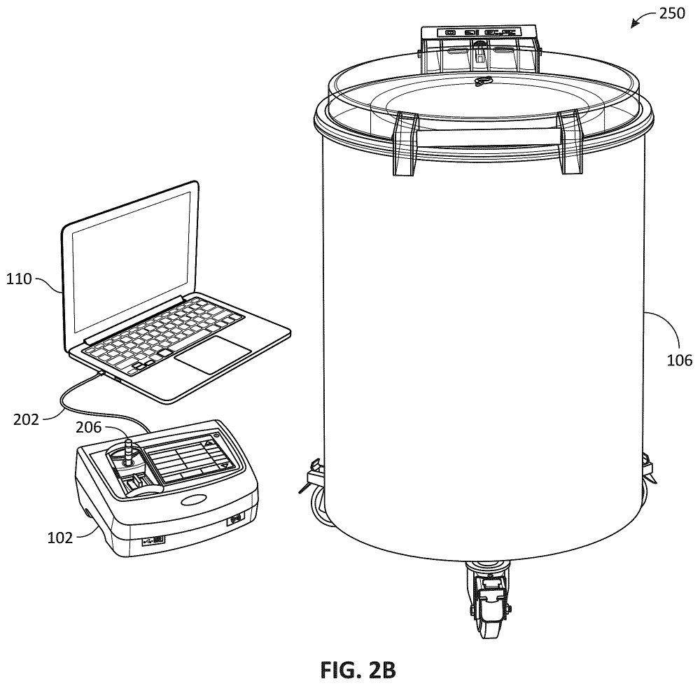

FIG. 2A depicts an example food processing environment 200 in some embodiments. The food processing environment 200 includes a food processing apparatus 106, a light intensity measuring apparatus 102, and a computing device 110 connected to the light intensity measuring apparatus 102 via a cable 202, which may be a Universal Serial Bus (USB) cable. The food processing apparatus 106 has pieces of produce 204, such as lettuce, on it to be washed. The light intensity measuring apparatus 102 and the computing device 110 may be positioned proximate to the food processing apparatus 106 and may be positioned on a support (e.g., a bench, a table, or the like, not illustrated in FIG. 2A).

The light intensity measuring apparatus 102 may perform scans of samples of food processing byproducts and obtain intensity measurements of a set of wavelengths of light that has passed through the samples. The samples may be placed in a cuvette 206 positioned in a receptacle of the light intensity measuring apparatus 102. The light intensity measuring apparatus 102 may convert the intensity measurements in the set of intensity measurements to other values, such as absorbance values, transmittance values, or concentration values, and thus obtain a first set of values based on the set of intensity measurements. The light intensity measuring apparatus 102 may transmit the first set of values to the computing device 110. In some embodiments, the light intensity measuring apparatus 102 transmits the set of intensity measurements to the computing device 110. In some embodiments, the light intensity measuring apparatus 102 measures the detected light in units other than intensity, such as in absorbance units or transmittance units, and transmits the measured other values to the computing device 110. In some embodiments, the light intensity measuring apparatus 102 transmits the first set of values to the foodborne pathogen detection system 104. In some embodiments, the light intensity measuring apparatus 102 transmits the first set of values to both the computing device 110 and to the foodborne pathogen detection system 104.

FIG. 2B depicts another example food processing environment 250 in some embodiments. In the food processing environment 250, the food processing apparatus 106 is a salad spinner that may be used to dry wet lettuce or other wet produce. The food processing apparatus 106 produces water as it spins, which may drain via one or more drain lines (not illustrated in FIG. 2B). Other like reference numerals in FIG. 2B refer to like elements in FIG. 2A and are not discussed with reference to FIG. 2B.

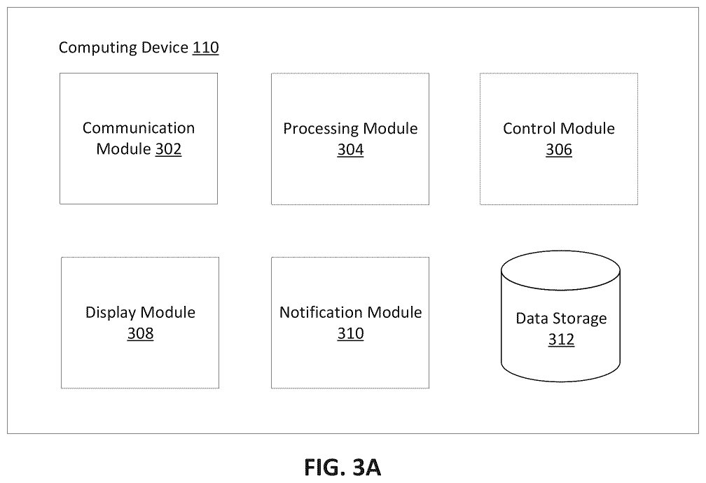

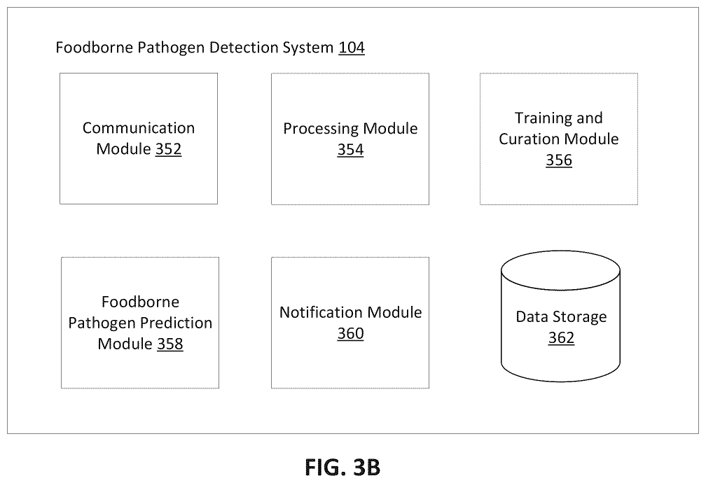

FIG. 3A is a block diagram of components of a computing device 110 in some embodiments. The computing device 110 includes a communication module 302, a processing module 304, a control module 306, a display module 308, a notification module 310, and a data storage 312.

The communication module 302 may send and/or receive requests and/or data between the computing device 110 and any of the food processing apparatuses 106, the foodborne pathogen detection system 104, and the light intensity measuring apparatuses 102. The communication module 302 may receive requests and/or data from the food processing apparatuses 106, the foodborne pathogen detection system 104, and/or the light intensity measuring apparatuses 102. The communication module 302 may also send requests and/or data to the food processing apparatuses 106, the foodborne pathogen detection system 104, and/or the light intensity measuring apparatuses 102.

The processing module 354 may process data, such as the first set of values received from the light intensity measuring apparatuses 102 and/or data received from the foodborne pathogen detection system 104 and/or the food processing apparatuses 106.

The control module 306 may control the light intensity measuring apparatus 102. In some embodiments, the light intensity measuring apparatus 102 includes an Application Programming Interface (API) or other interface by which the light intensity measuring apparatus 102 may be controlled, and the control module 306 controls the light intensity measuring apparatus 102 using the API or the other interface.

The display module 308 displays user interfaces for the computing device 110. The notification module 310 may provide reports, alerts, and/or dashboards that include results, confidence scores, and/or other information. For example, the computing device 110 may track foodborne pathogen detections on particular food processing equipment as well as what food was processed on the food processing equipment. As another example, the computing device 110 may track foodborne pathogen detections in certain parts of a food processing facility as well as what food was processed in those certain parts. The computing device 110 may thus be able to identify food (e.g., particular lots or production runs) and recommend, via the notification module 310, that remedial action, such as quarantining food, recalling food, or other action, should be taken. The notification module 310 may optionally notify appropriate third parties (e.g., government agencies such as the U.S. FDA) of the detection of foodborne pathogens. The notification module 310 may, in some embodiments, prepare reports to aid in compliance with food safety laws and regulations.

The data storage 312 may include data stored, accessed, and/or modified by any of the modules of the computing device 110. The data storage 312 may include any number of data storage structures such as tables, databases, lists, and/or the like.

FIG. 3B depicts components of a block diagram of the foodborne pathogen detection system 104 in some embodiments. The foodborne pathogen detection system 104 includes a communication module 352, a processing module 354, a training and curation module 356, a foodborne pathogen prediction module 358, a notification module 360, and a data storage 362.

The communication module 352 may send and/or receive requests and/or data between the foodborne pathogen detection system 104 and any of the food processing apparatuses 106, the computing devices 110, and the light intensity measuring apparatuses 102. The communication module 352 may receive requests and/or data from the food processing apparatuses 106, the computing devices 110, and/or the light intensity measuring apparatuses 102. The communication module 352 may also send requests and/or data to the food processing apparatuses 106, the computing devices 110, and/or the light intensity measuring apparatuses 102.

The processing module 354 may process the first set of values received from the light intensity measuring apparatuses 102 via the computing devices 110 to obtain a second set of values.

The training and curation module 356 may train an artificial intelligence and/or machine learning system, (e.g., such as a set of decision trees) to be applied to the second set of values.

The foodborne pathogen prediction module 358 may apply the trained artificial intelligence and/or machine learning system, such as the set of trained decision trees, to the second set of values to obtain a result. A result may indicate either a positive (a positive foodborne pathogen detection) or a negative (a negative foodborne pathogen detection) for a foodborne pathogen for the sample of the food processing byproduct.

The notification module 360 may generate and provide notifications that include results of foodborne pathogen detections of the sample as well as other information, such as a confidence score. The notification module 360 may provide reports, alerts, and/or dashboards that include results, confidence scores, and/or other information. For example, the foodborne pathogen detection system 104 may track foodborne pathogen detections on particular food processing equipment as well as what food was processed on the food processing equipment. As another example, the foodborne pathogen detection system 104 may track foodborne pathogen detections in certain parts of a food processing facility as well as what food was processed in those certain parts. The pathogen detection system 104 may thus be able to identify food (e.g., particular lots or production runs) and recommend, via the notification module 360, that remedial action, such as quarantining food, recalling food, or other action, should be taken. The notification module 360 may optionally notify appropriate third parties (e.g., government agencies such as the U.S. FDA) of the detection of foodborne pathogens. The notification module 360 may, in some embodiments, prepare reports to aid in compliance with food safety laws and regulations.

The data storage 362 may include data stored, accessed, and/or modified by any of the modules of the foodborne pathogen detection system 104. The data storage 362 may include any number of data storage structures such as tables, databases, lists, and/or the like.

A module of the computing device 110 or the foodborne pathogen detection system 104 may be hardware, software, firmware, or any combination. For example, each module may include functions performed by dedicated hardware (e.g., an Application-Specific Integrated Circuit (ASIC), a Field Programmable Gate Array (FPGA), or the like), software, instructions maintained in ROM, and/or any combination. Software may be executed by one or more processors. Although a limited number of modules are depicted in FIGS. 3A and 3B, there may be any number of modules. Further, individual modules may perform any number of functions, including functions of multiple modules as shown herein. Further, modules depicted as being included in the computing device 110 may be additionally or alternatively included in the foodborne pathogen detection system 104, and modules included in the foodborne pathogen detection system 104 may be additionally or alternatively included in the computing device 110.

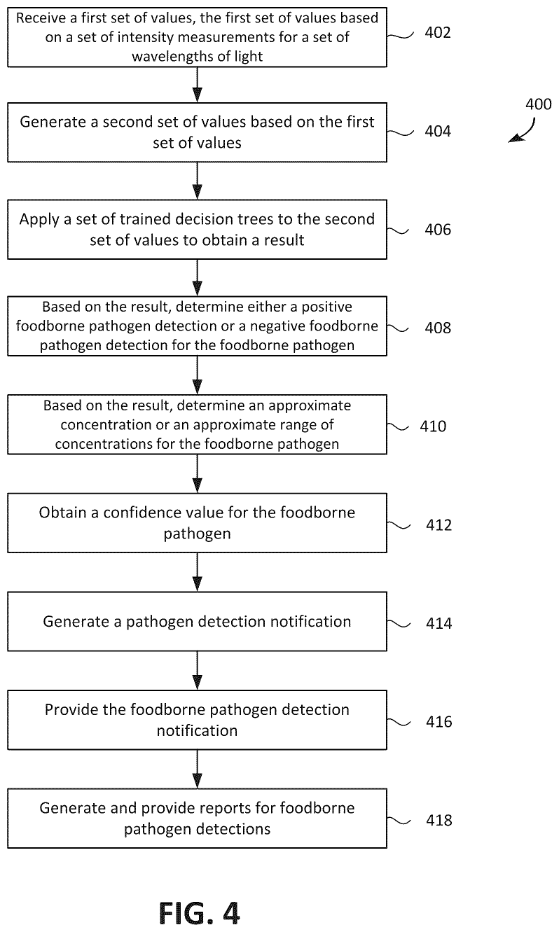

FIG. 4 is a flowchart showing a method 400 for detecting foodborne pathogens in some embodiments. Various modules of the foodborne pathogen detection system 104 perform the method 400. In some embodiments, various modules of the computing device 110 performs the method 400. In some embodiments, various modules of both the computing device 110 and the foodborne pathogen detection system 104 perform the method 400.

In some embodiments, a person working in a food processing facility in which the food processing apparatus 106 is located fills the cuvette 206 or other suitable container with a sample of food processing byproduct and places the cuvette 206 in an appropriate receptacle of the light intensity measuring apparatus 102. The person may fill the cuvette 206 periodically, as needed, for each lot or shipment of food to be processed, or on a predetermined schedule. It will be understood that samples of food processing byproducts may be tested at various times. In some embodiments, the cuvette 206 may be filled by an automated device or system without intervention by a person. In some embodiments, the sample of food processing byproduct may be mixed with a reagent. In some embodiments, the sample of food processing byproduct may be mixed with a neutral or inert substance.

After filling the cuvette 206, the person may then start a scan of the sample of the food processing byproduct using an interface of the light intensity measuring apparatus 102. Additionally or alternatively, the person may start the scan using the computing device 110, which may control the light intensity measuring apparatus 102. The light intensity measuring apparatus 102 generates light that passes through at least a portion of the sample of the food processing byproduct in the cuvette 206 and detects the light that has passed through at least the portion of the sample of the food processing byproduct in the cuvette 206. The light intensity measuring apparatus 102 measures intensities of the detected light for a set of wavelengths of the light and obtains a set of intensity measurements for the set of wavelengths of the light. In some embodiments, the set of wavelengths of light includes wavelengths of light in the ultraviolet, visible, and infrared spectrums. In some embodiments, the set of wavelengths of light includes wavelengths of light ranging from approximately 300 nanometers (nm) (for example, approximately 320 nm) to approximately 1100 nm (for example, approximately 1100 nm). In some embodiments, the light intensity measuring apparatus 102 has a resolution of 1 nm and obtains a set of 781 intensity measurements for a set of 781 wavelengths of the light.

The method 400 may begin at step 402, where the communication module 352 receives a first set of values from the light intensity measuring apparatus 102 via the computing device 110. The first set of values are based on the set of intensity measurements for a set of wavelengths of light that the light intensity measuring apparatus 102 obtained.

At step 404, the processing module 354 generates a second set of values based on the first set of values. In some embodiments, the processing module 354 normalizes each value in the second set of values to be between zero, inclusive, and one, inclusive. The processing module 354 may further process the values in the second set of values. The processing module 354 may generate the second set of values using other techniques, such as applying a fitting function to each value in the first set of values to generate each value in the second set of values.

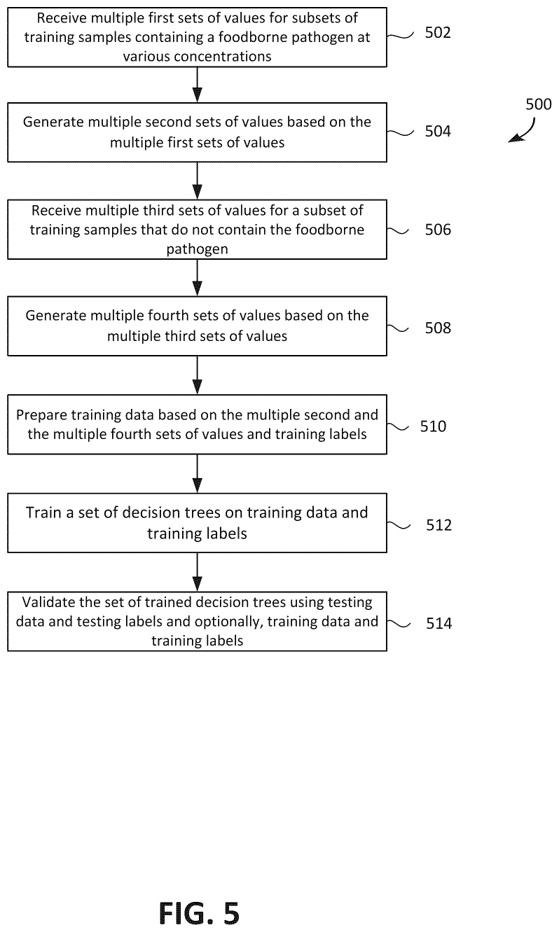

At step 406, the foodborne pathogen prediction module 358 applies a set of trained decision trees to the second set of values to obtain a result. As discussed further herein with reference to, for example, FIG. 5, the training and curation module 356 trains a set of decision trees on a set of training samples. A first subset of training samples of the set of training samples contain a foodborne pathogen at a first concentration and a second subset of training samples of the set of training samples contain the foodborne pathogen at a second concentration different from the first concentration. In some embodiments, the foodborne pathogen prediction module 358 utilizes the following Python code to apply the set of trained decision trees to the set of values to obtain a result:

y_pred=model.predict(x_test)

In this code, y_pred is the result and x_test is testing data. In some embodiments, the set of trained decision trees may operate in a binary mode. In such embodiments, the result may be a float that has a value between zero, inclusive, and one, inclusive. In some embodiments, the set of trained decision trees may operate in a multiclass mode. In such embodiments, the result may be an integer that has value of either zero, one, or another integer value greater than one.

At step 408, the foodborne pathogen prediction module 358, based on the result, determines either a positive foodborne pathogen detection or a negative foodborne pathogen detection for the foodborne pathogen in the sample of the food processing byproduct. In some embodiments, the foodborne pathogen prediction module 358 determines that the result indicates a positive foodborne pathogen detection if the result meets or exceeds a threshold, and that the result indicates a negative foodborne pathogen detection if the result does not meet or exceed the threshold. In embodiments where the result is a float value between zero, inclusive, and one, inclusive, the threshold may be 0.5. In embodiments where the result is an integer with a value of either zero, one, or another integer value greater than one, zero indicates a negative result and one or another integer value greater than one indicates a positive result. As discussed in more detail herein, in such embodiments, the result may indicate both a positive foodborne pathogen detection as well as a concentration of the foodborne pathogen in the sample of the food processing byproduct.

At step 410, the foodborne pathogen prediction module 358, based on the result, determines an approximate concentration or an approximate range of concentrations for the foodborne pathogen in the sample of the food processing byproduct.

At step 412, the foodborne pathogen prediction module 358 obtains a confidence value for the foodborne pathogen in the sample of the food processing byproduct. In some embodiments, the foodborne pathogen prediction module 358 utilizes the following Python code to obtain the confidence score:

y_score=model.predict_proba(x_test)

In this code, y_score is the confidence value, which may be a float that ranges between zero, inclusive, and one, inclusive. The closer the value is to zero the higher the degree of confidence that the result is negative, and the closer the value is to one the higher the degree of confidence that the result is positive. In some embodiments, the confidence value may be expressed as a percentage between 0% and 100%, inclusive.

At step 414, the notification module 360 generates a foodborne pathogen detection notification that indicates either the positive foodborne pathogen detection or the negative foodborne pathogen detection for the foodborne pathogen in the sample of the food processing byproduct. In some embodiments, the foodborne pathogen detection notification further indicates the approximate concentration or the approximate range of concentrations for the foodborne pathogen in the sample of the food processing byproduct. In some embodiments, the foodborne pathogen detection notification further indicates the confidence value for the foodborne pathogen in the sample of the food processing byproduct. In some embodiments, if the confidence value is within a certain range or above or below a certain threshold, the foodborne pathogen detection notification may include a flag indicating such. For example, if the confidence value is below a certain threshold, the foodborne pathogen detection notification may flag that there is low confidence in the result. As another example, if the confidence value is above a certain threshold, the foodborne pathogen detection notification may flag that there is high confidence in the result.

At step 416, the notification module 360 provides the foodborne pathogen detection notification. In some embodiments, the notification module 360 provides the foodborne pathogen detection notification to the computing device 110 for display by the display module 308. The notification module 360 may provide the foodborne pathogen detection notification for display by other digital devices. At step 418, the notification module 360 generates and provides reports, such as dashboards, spreadsheets, or the like, that include results, confidence scores, and/or other information.

In some embodiments, the set of trained decision trees is a first set of trained decision trees, the set of training samples is a first set of training samples, the foodborne pathogen is a first foodborne pathogen, the result is a first result, the positive foodborne pathogen detection is a first positive foodborne pathogen detection, the negative foodborne pathogen detection is a first negative foodborne pathogen detection, and the foodborne pathogen detection notification is a first foodborne pathogen detection notification. In such embodiments, the foodborne pathogen prediction module 358 may apply a second set of trained decision trees to the second set of values to obtain a second result. The second set of trained decision trees may be trained on a second set of training samples, where a third subset of training samples of the second set of training samples contain a second foodborne pathogen at a third concentration and a fourth subset of training samples of the second set of training samples contain the second foodborne pathogen at a fourth concentration different from the third concentration. The second foodborne pathogen is different from the first foodborne pathogen. For example, the first foodborne pathogen may be E. coli and the second foodborne pathogen may be salmonella.

Further in such embodiments, the foodborne pathogen prediction module 358 may, based on the second result, determine either a second positive foodborne pathogen detection or a second negative foodborne pathogen detection for the second foodborne pathogen in the sample of the food processing byproduct. The foodborne pathogen prediction module 358 may also generate a second foodborne pathogen detection notification that indicates either the second positive foodborne pathogen detection or the second negative foodborne pathogen detection for the second foodborne pathogen in the sample of the food processing byproduct. The notification module 360 may also provide the second foodborne pathogen detection notification.

In some embodiments, the set of intensity measurements for the set of wavelengths of light is a first set of intensity measurements for the set of wavelengths of light and the result is a first result. In such embodiments, the communication module 302 may receive at least one second set of values from the computing device 110. The at least one second set of values are based on at least one second set of intensity measurements for the set of wavelengths of light obtained by the light intensity measuring apparatus 102. For example, the light intensity measuring apparatus 102 may perform multiple scans of the sample of the food processing byproduct and provide the multiple sets of values to the foodborne pathogen detection system 104 via the computing device 110. The foodborne pathogen detection system 104 may perform step 404 to step 406 of the method 400 and obtain multiple results.

Further in such embodiments, the foodborne pathogen detection system 104 may then determine either the positive foodborne pathogen detection or the negative foodborne pathogen detection for the foodborne pathogen in the sample of the food processing byproduct based on the multiple results. For example, if the foodborne pathogen detection system 104 obtains three results, the foodborne pathogen detection system 104 may determine either the positive foodborne pathogen detection or the negative foodborne pathogen detection for the foodborne pathogen in the sample of the food processing byproduct based on the two results that have the highest confidence score. As another example, if the foodborne pathogen detection system 104 obtains three results, the foodborne pathogen detection system 104 may determine either the positive foodborne pathogen detection or the negative foodborne pathogen detection for the foodborne pathogen in the sample of the food processing byproduct based on the best two results of the three results. It will be understood that the foodborne pathogen detection system 104 may determine either the positive foodborne pathogen detection or the negative foodborne pathogen detection for the foodborne pathogen in the sample of the food processing byproduct in various ways.

FIG. 5 is a flowchart showing a method 500 for training sets of decision trees for detecting foodborne pathogens in some embodiments. Various modules of the foodborne pathogen detection system 104 perform the method 500. In some embodiments, various modules of the computing device 110 performs the method 500. In some embodiments, various modules of both the computing device 110 and the foodborne pathogen detection system 104 perform the method 500.

In some embodiments, a person, such as a laboratory technician, prepares a set of training samples. A foodborne pathogen, such as E. coli, may be cultivated in a solution, such as tryptic soy broth (TSB) solution. The initial concentration of the foodborne pathogen in the solution may be approximately 1e⁸ colony-forming units/milliliter (cfu/mL). In some embodiments, the initial concentration of the foodborne pathogen in the solution may range from approximately 1e⁶ to approximately 1e⁸ cfu/mL. In some embodiments, the initial concentration of the foodborne pathogen in the solution may be greater than approximately 1e⁸ cfu/mL. In some embodiments, the initial concentration of the foodborne pathogen in the solution may be lower than approximately 1e⁸ cfu/mL.

A first subset of training samples at the initial concentration of approximately 1e⁸ cfu/mL may be prepared. A second subset of training samples may be prepared that have been diluted 10:1 from the initial concentration using the solution, so as to have a second concentration of approximately 1e⁷ cfu/mL. A third subset of training samples may be prepared that have been diluted 100:1 from the initial concentration using the solution, so as to have a third concentration of approximately 1e⁶ cfu/mL. A fourth subset of training samples may be prepared that have been diluted 1000:1 from the initial concentration using the solution, so as to have a fourth concentration of approximately 1e⁵ cfu/mL. A fifth subset of training samples may be prepared that have been diluted 10,000:1 from the initial concentration using the solution, so as to have a fifth concentration of approximately 1e⁴ cfu/mL. A sixth subset of training samples may be prepared that have been diluted 100,000:1 from the initial concentration using the solution, so as to have a sixth concentration of approximately lea cfu/mL. A seventh subset of training samples may be prepared that have been diluted 1,000,000:1 from the initial concentration using the solution, so as to have a seventh concentration of approximately 1e² cfu/mL. An eighth subset of training samples may be prepared that have been diluted from the initial concentration using the solution, so as to have an eighth concentration of approximately 1 cfu/mL. A ninth subset of training samples may be prepared that contain only the solution, for example, the TSB solution.

In some embodiments, there may be fewer than or more than eight subsets of training samples at different concentrations. In some embodiments, the different subsets of training samples may be diluted using different dilution ratios to obtain different concentrations than those described herein. In some embodiments, the set of training samples contains approximately 2000 training samples that include a foodborne pathogen, which may be referred to herein as positive training samples. In some embodiments, the set of training samples contains fewer than 2000 positive training samples. In some embodiments, the set of training samples contains more than 2000 positive training samples. In some embodiments, the set of training samples contains approximately the same number of training samples that do not include a foodborne pathogen, which may be referred to herein as negative training samples, as the number of positive training samples. In some embodiments, the number of negative training samples is less than the number of positive training samples. In some embodiments, the number of negative training samples is more than the number of positive training samples.

In some embodiments, the set of training samples are prepared prior to the light intensity measuring apparatus 102 scanning the training samples. In some embodiments, the first subset of training samples at the initial concentration of approximately 1e⁸ cfu/mL are prepared and scanned by the light intensity measuring apparatus 102. Then, the first subset of training samples are diluted 10:1 from the initial concentration to obtain the second subset of training samples at the second concentration of approximately 1e⁷ cfu/mL, and then the second subset of training samples are scanned by the light intensity measuring apparatus 102. This dilution and scanning may be repeated several times to obtain, and then scan, the third through eighth subsets of training samples.

To scan a training sample, the cuvette 206 or other suitable container may be filled with a training sample and placed in an appropriate receptacle of the light intensity measuring apparatus 102. The person may then start a scan of the sample of the food processing byproduct using an interface of the light intensity measuring apparatus 102. Additionally or alternatively, the person may start the scan using a computing device 110, which may control the light intensity measuring apparatus 102. The light intensity measuring apparatus 102 generates light that passes through at least a portion of the training sample in the cuvette 206 and detects the light that has passed through at least the portion of the training sample in the cuvette 206. The light intensity measuring apparatus 102 measures intensities of the light for a set of wavelengths of the light and obtains a set of intensity measurements for the set of wavelengths of the light. In some embodiments, the set of wavelengths of light includes wavelengths of light in the ultraviolet, visible, and infrared spectrums. In some embodiments, the set of wavelengths of light includes wavelengths of light ranging from approximately 300 nanometers (nm) (for example, approximately 320 nm) to approximately 1100 nm (for example, approximately 1100 nm). In some embodiments, the light intensity measuring apparatus 102 has a resolution of 1 nm and obtains a set of 781 intensity measurements for a set of 781 wavelengths of the light.

The method 500 begins at step 502 where the communication module 352 receives multiple first sets of values from the light intensity measuring apparatus 102 via the computing device 110 based on multiple scans of the first subset of training samples containing the foodborne pathogen at the first concentration. The multiple first set of values are based on multiple sets of intensity measurements for a set of wavelengths of light that the light intensity measuring apparatus 102 obtained for scans of the first subset of training samples containing the foodborne pathogen at the first concentration.

Step 502 may be performed for each subset of training samples containing the foodborne pathogen at a different concentration. That is, the communication module 352 may perform step 502 for the first subset of training samples at the first concentration of approximately 1e⁸ cfu/mL, for the second subset of training samples at the second concentration of approximately 1e⁷ cfu/mL, up to and including for the eighth subset of training samples at the eighth concentration of approximately 1 cfu/mL.

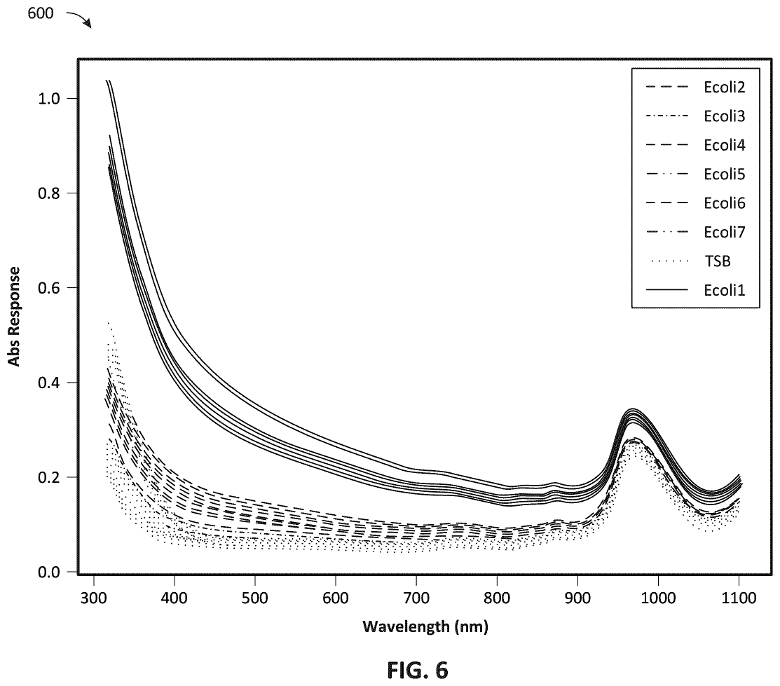

FIG. 6 depicts a graph 600 of absorption for a set of wavelengths for multiple instances of light that passed through multiple samples containing different concentrations of E. coli and multiple samples that do not contain E. coli in some embodiments. The wavelengths of light range from approximately 320 nm to approximately 1100 nm. The graph 600 shows the absorption by wavelength for multiple training samples containing E. coli at a first concentration (Ecoli1), multiple training samples containing E. coli at a second concentration lower than the first concentration (Ecoli2), multiple training samples containing E. coli at a third concentration lower than the second concentration (Ecoli3), multiple training samples containing E. coli at a fourth concentration lower than the third concentration (Ecoli4), multiple training samples containing E. coli at a fifth concentration lower than the fourth concentration (Ecoli5), multiple training samples containing E. coli at a sixth concentration lower than the fifth concentration (Ecoli6), multiple training samples containing E. coli at a seventh concentration lower than the sixth concentration (Ecoli7), and multiple training samples that do not contain E. coli (TSB). The graph 600 illustrates that the absorption across the range of wavelengths generally decreases as the concentration of E. coli in a training sample decreases, or conversely, that the absorption generally increases as the concentration of E. coli in a training sample increases.

Returning to FIG. 5, at step 504, the processing module 354 generates multiple second sets of values based on the multiple first sets of values. In some embodiments, the processing module 354 normalizes each value in the multiple second sets of values to be between zero, inclusive, and one, inclusive. The processing module 354 may further process the values in the multiple second sets of values. The processing module 354 may generate the multiple second sets of values using other techniques, such as applying a fitting function to each value in the multiple first sets of values to generate each value in the multiple second sets of values.

Step 504 may be performed for each subset of training samples containing the foodborne pathogen at a different concentration. That is, the processing module 354 may perform step 504 for the first subset of training samples at the first concentration of approximately 1e⁸ cfu/mL, for the second subset of training samples at the second concentration of approximately 1e⁷ cfu/mL, up to and including for the eighth subset of training samples at the eighth concentration of approximately 1 cfu/mL.

At step 506, the communication module 352 receives multiple third sets of values from the light intensity measuring apparatus 102 via the computing device 110 based on multiple scans of the subset of training samples that do not contain the foodborne pathogen. At step 508 the processing module 354 generates multiple fourth sets of values based on the multiple third sets of values. In some embodiments, the processing module 354 normalizes each value in the multiple fourth sets of values to be between zero, inclusive, and one, inclusive. The processing module 354 may further process the values in the multiple fourth sets of values. The processing module 354 may generate the multiple fourth sets of values using other techniques, such as applying a fitting function to each value in the multiple third sets of values to generate each value in the multiple fourth sets of values.

At step 510, the training and curation module 356 prepares training data based on the multiple second sets of values and the multiple fourth sets of values. The training and curation module 356 also prepares training labels for the training data. In embodiments where the set of trained decision trees operate in a binary mode, a training label may be either a zero (0) for a negative training sample and a one (1) for a positive training sample. In embodiments where the set of trained decision trees operate in a multiclass mode, a training label may be either a zero (0) for a negative training sample, a one (1) for a positive training sample having a foodborne pathogen concentration at a first concentration, a two (2) for a positive training sample having a foodborne pathogen concentration at a second concentration, a three (3) for a positive training sample having a foodborne pathogen concentration at a third concentration, a four (4) for a positive training sample having a foodborne pathogen concentration at a fourth concentration, a five (5) for a positive training sample having a foodborne pathogen concentration at a fifth concentration, a six (6) for a positive training sample having a foodborne pathogen concentration at a sixth concentration, a seven (7) for a positive training sample having a foodborne pathogen concentration at a seventh concentration, and an eight (8) for a positive training sample having a foodborne pathogen concentration at an eighth concentration. In some embodiments, there are fewer than eight different concentrations of the foodborne pathogen in the training samples and a corresponding lower number of different training labels. In some embodiments, there are more than eight different concentrations of the foodborne pathogen in the training samples and a corresponding higher number of different training labels.

At step 512, the training and curation module 356 trains a set of decision trees for the foodborne pathogen. In some embodiments, the training and curation module 356 utilizes an optimized distributed gradient boosting library, XGBoost. In some embodiments, the training and curation module 356 utilizes the following Python code to create each set of decision trees:

```
from xgboost import XGBClassifier
params = {“booster”: “gbtree”,
  “objective”:“binary:logistic”,
  “max_delta_step”:20,
  “eval_metric”:“error”,
  “n_estimators”:10000,
  “verbosity”:0,
  “max_depth”:500,}
  self.config.params = params
model = XGBClassifier(**params)
```

XGBClassifier may be understood as a single model that is an ensemble of 10,000 decision trees (the “n_estimators”:10000 parameter). In some embodiments, the training and curation module 356 may utilize parameters other than or in addition to those listed herein. In some embodiments, the training and curation module 356 may utilize different values for model parameters than those listed herein.

In some embodiments, the training and curation module 356 utilizes the following Python code to train each set of the multiple sets of decision trees:
model.fit(x_train, y_train, eval_set=[(x_train, y_train), (x_test, y_test)], early_stopping_rounds=50)
   In this code, x_train is training data, y_train is training labels, x_test is testing data, and y_test is testing labels. Both x_train and x_test are ground truth data. Both x_train and x_test may include both positive training samples and negative training samples. In some embodiments, both the x_train and x_test data are balanced, meaning that they include equal or generally equal numbers of positive training samples and negative training samples. In some embodiments, the x_train and x_test data may be imbalanced toward negative training samples, meaning that they include more negative training samples than positive training samples. The training and curation module 356 may also use data sets that are imbalanced towards positive training samples, meaning that they include more positive training samples than negative training samples.

The set of trained decision trees may operate in a binary mode or a multiclass mode. The following Python code may be utilized to determine which mode the set of trained decision trees may operate in:

```
# if binary, set binary, otherwise, set multiclass
if np.max(y_train) == 1:
  params[‘objective’] = “binary:logistic”
  params[‘eval_metric’] = ‘error’
elif np.max(y_train) > 1:
  params[‘objective’] = “multi:softmax”
  params[‘eval_metric’] = ‘merror’
```

At step 514, the training and curation module 356 validates the set of trained decision trees. In some embodiments, the training and curation module 356 utilizes both training data and testing data to validate the set of trained decision trees. In some embodiments, the training and curation module 356 utilizes only testing data to validate the set of trained decision trees.

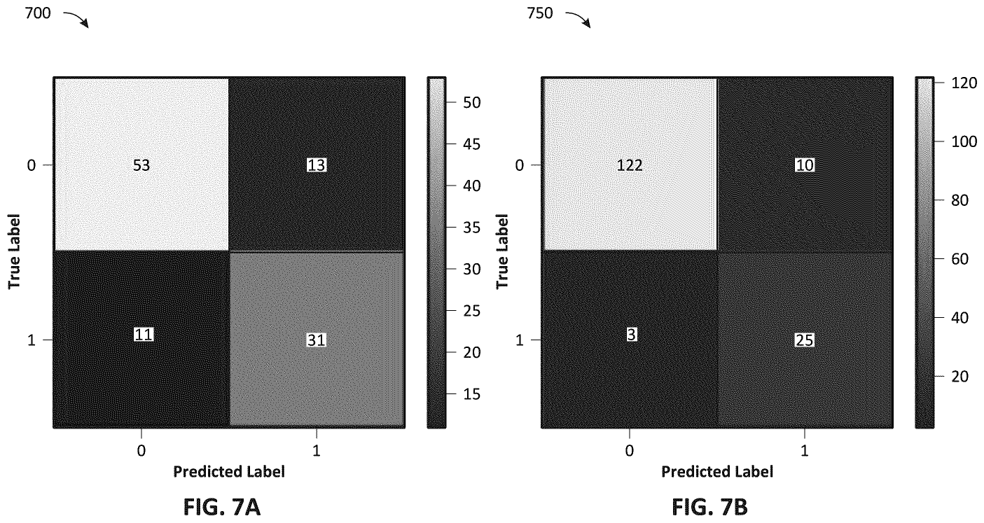

FIG. 7A depicts a confusion matrix 700 for results of applications of a set of trained decision trees to multiple sets of values based on intensity measurements of multiple instances of light that has passed through multiple testing samples containing E. coli at seven different concentrations and multiple testing samples that do not contain E. coli in some embodiments. The set of trained decision trees that produced the results in the confusion matrix 700 operated in a binary mode and there were 108 total testing samples. In the set of testing samples, there were different testing samples containing seven different concentrations of E. coli and testing samples that did not contain E. coli. The upper left hand quadrant of the confusion matrix 700 indicates true negatives, of which there are 53. The upper right hand quadrant of the confusion matrix 700 indicates false negatives, of which there are 13. The lower left hand quadrant of the confusion matrix 700 indicates false positives, of which there are 11. The lower right hand quadrant of the confusion matrix 700 indicates true positives, of which there are 31. The confusion matrix indicates that the set of trained decision trees is 77% accurate, with 80% specificity and 74% sensitivity. This indicates that the set of trained decision trees would detect E. coli 74% of the time in samples containing E. coli at the lowest concentration (the seventh concentration, which may be approximately 1.5 cfu/mL) and higher concentrations (for example, concentrations at approximately 1.5e² cfu/mL to approximately 1.5e⁷ cfu/mL).

FIG. 7B depicts a confusion matrix 750 for results of applications of a set of trained decision trees to multiple sets of values based on intensity measurements of multiple instances of light that has passed through multiple testing samples containing E. coli at four different concentrations and multiple testing samples that do not contain E. coli in some embodiments. The set of trained decision trees that produced the results in the confusion matrix 750 operated in a binary mode and there were 160 total testing samples. In the set of testing samples, there were different testing samples containing four different concentrations of E. coli and testing samples that did not contain E. coli. The upper left hand quadrant of the confusion matrix 750 indicates true negatives, of which there are 122. The upper right hand quadrant of the confusion matrix 750 indicates false negatives, of which there are 10. The lower left hand quadrant of the confusion matrix 750 indicates false positives, of which there are 3. The lower right hand quadrant of the confusion matrix 750 indicates true positives, of which there are 25. The confusion matrix 750 indicates that the set of trained decision trees is 92% accurate, with 92% specificity and 89% sensitivity. This indicates that the set of trained decision trees would detect E. coli 89% of the time in samples containing E. coli at the lowest concentration (the fourth concentration, which may be approximately 1.5e⁴ cfu/mL) and higher concentrations (for example concentrations at approximately 1.5e⁵ cfu/mL to approximately 1.5e⁷ cfu/mL).

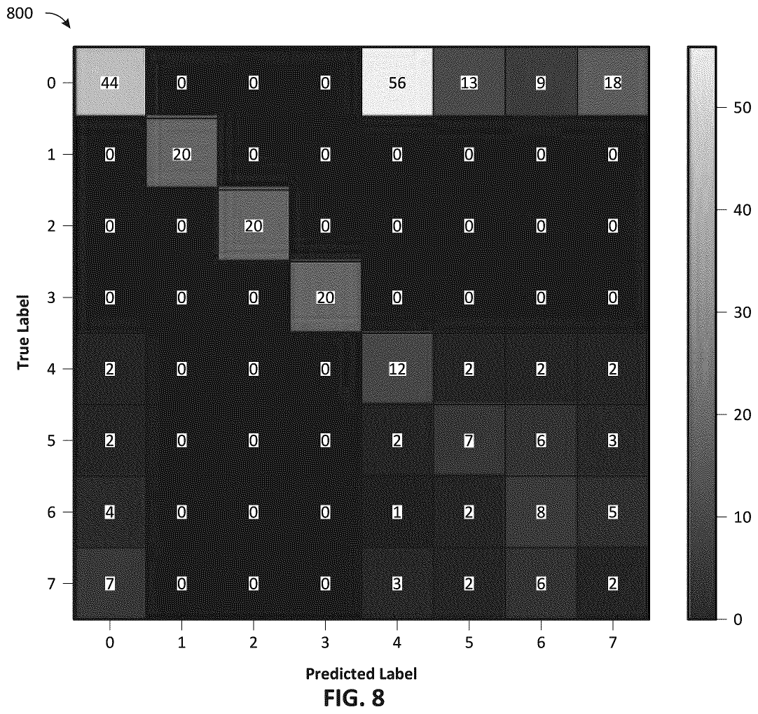

FIG. 8 depicts a confusion matrix 800 for results of applications of a set of trained decision trees to multiple sets of values based on intensity measurements of multiple instances of light that has passed through multiple testing samples containing E. coli at seven different concentrations and multiple testing samples that do not contain E. coli in some embodiments. The set of trained decision trees that produced the results in the confusion matrix 700 operated in a multiclass mode and there were 280 total testing samples. In the set of testing samples, there were different testing samples containing seven different concentrations of E. coli and testing samples that did not contain E. coli. The confusion matrix 800 indicates that the set of trained decision trees operating in the multiclass mode accurately identifies the concentration of a testing sample up to the third concentration (for example, a concentration at approximately 1.5e⁴ cfu/mL). The confusion matrix 800 further indicates that the set of trained decision trees operating in the multiclass mode accurately identifies an approximate range of concentrations for the fourth concentrations through the seventh concentrations.

In some embodiments, the training and curation module 356 performs the method 500 for each of multiple foodborne pathogens. That is, the training and curation module 356 trains a set of decision trees for each of multiple foodborne pathogens, such as E. coli, salmonella, and listeria. In some embodiments, the training and curation module 356 may train a set of decision trees for each of the following foodborne pathogens: norovirus, salmonella (non-typhoidal), Clostridium perfringens, campylobacter, Staphylococcus aureus, Toxoplasma gondii, Escherichia coli (E. coli), Clostridium botulinum, cryptosporidium, Cyclospora, hepatitis A virus, shigella, Yersinia, and Listeria monocytogenes (listeria). The foodborne pathogen prediction module 358 may apply one or more of the trained sets of decision trees to detect foodborne pathogens. Accordingly, the foodborne pathogen detection system 104 may provide panel detection and notification for various foodborne pathogens. One advantage of the foodborne pathogen detection system 104 is that it may provide results for such panel tests quickly (e.g., within seconds or minutes). Another advantage of the foodborne pathogen detection system 104 is that it obviates the need for sending samples to laboratories for test, which may reduce logistical issues and/or complexity.

In various embodiments, a machine learning and/or AI architecture may be utilized (e.g., random forest, statistical approaches, and/or the like) in addition to or as an alternative to the sets of decision trees discussed herein. The machine learning and/or AI architecture may utilize the features discussed herein to generate predictive models and/or make predictions. In various embodiments, a 1d or 2d convolutional neural network (CNN) may be used as a discriminator to identify measurements indicating foodborne pathogen contamination and non-foodborne pathogen contamination. In various embodiments, a neural network may be trained using measurements or values from the light intensity measuring apparatuses 102 as discussed herein. The neural network may also be trained using laboratory test results to confirm those foods, equipment, and/or surfaces that are contaminated and those that are not contaminated. The neural network may receive or generate a set of features based on the output (i.e., measurement results or values based thereon) of the light intensity measuring apparatuses 102. The neural network may then be tested to confirm predictions against known foodborne pathogen contamination and non-foodborne pathogen contamination results. In various embodiments, the models may utilize time series data generated by the light intensity measuring apparatuses 102 to make determinations about foodborne pathogen contamination.

In various embodiments, the training and curation module 356 may receive new ground truth data for a particular foodborne pathogen (e.g., new data that includes both positive samples and negative samples for the particular foodborne pathogen) and update the training data and the testing data and retrain the set of decision trees corresponding to the particular foodborne pathogen. For example, the training and curation module 356 may receive new ground truth data for salmonella. The training and curation module 356 may then update the training data and the testing data for salmonella and retrain the set of decision trees for salmonella. This may allow the foodborne pathogen detection system 104 to better detect salmonella in samples of food processing byproducts. The foodborne pathogen detection system 104 may utilize similar processes for other foodborne pathogens such as E. coli and listeria. As a result, the models and/or AI architecture may be updated, improved, and/or curated based on new positive samples and new negative samples in the new ground truth data.

In some embodiments, the training and curation module 356 may train sets of decision trees using training samples from a particular food processing facility, a region that includes multiple food processing facilities, or one or more classes of food processing facilities. This may allow the training and curation module 356 to create sets of trained decision trees that are customized for a particular food processing facility (for example, a single food processing facility), a particular location (for example, food processing facilities located in the Central Valley of California), or a particular type of food processing facility (for example, food processing facilities that slaughter chickens and process slaughtered chickens).

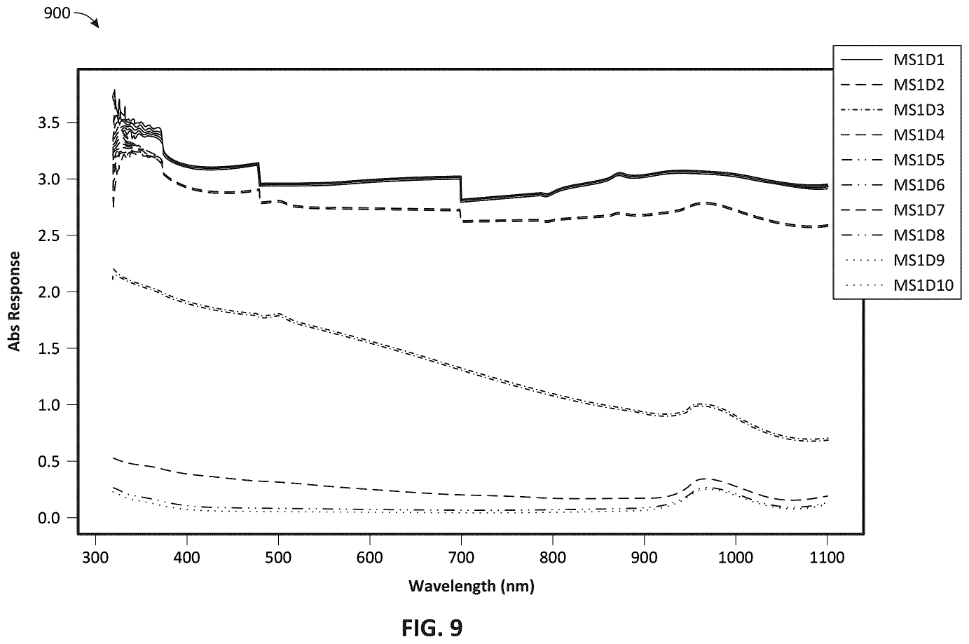

FIG. 9 depicts a graph 900 of absorption for a set of wavelengths for multiple instances of light that passed through multiple samples containing different concentrations of microspheres in some embodiments. The wavelengths of light range from approximately 320 nm to approximately 1100 nm. The graph 900 shows the absorption by wavelength for multiple samples containing—1-micron dragon green (480 nm-520 nm) microspheres at a first concentration of approximately 2.07e¹⁰ microspheres/mL (MS1D1), multiple training samples containing microspheres at a second concentration of approximately 2.07e⁹ microspheres/mL (MS1D2), multiple training samples containing microspheres at a third concentration of approximately 2.07e⁸ microspheres/mL (MS1D3), multiple training samples containing microspheres at a fourth concentration of approximately 2.07e⁷ microspheres/mL (MS1D4), multiple training samples containing microspheres at a fifth concentration of approximately 2.07e⁶ microspheres/mL (MS1D5), multiple training samples containing microspheres at a sixth concentration of approximately 2.07e⁵ microspheres/mL (MS1D6), multiple training samples containing microspheres at a seventh concentration of approximately 2.07e⁴ microspheres/mL (MS1D7), multiple training samples containing microspheres at an eighth concentration of approximately 2.07e³ microspheres/mL (MS1D8), multiple training samples containing microspheres at a ninth concentration of approximately 2.07e² microspheres/mL (MS1D9), and multiple training samples containing microspheres at a tenth concentration of approximately 2.07 microspheres/mL (MS1D10). The graph 900 illustrates that the absorption across the range of wavelengths generally decreases as the concentration of microspheres in a sample decreases, or conversely, that the absorption generally increases as the concentration of microspheres in a sample increases.

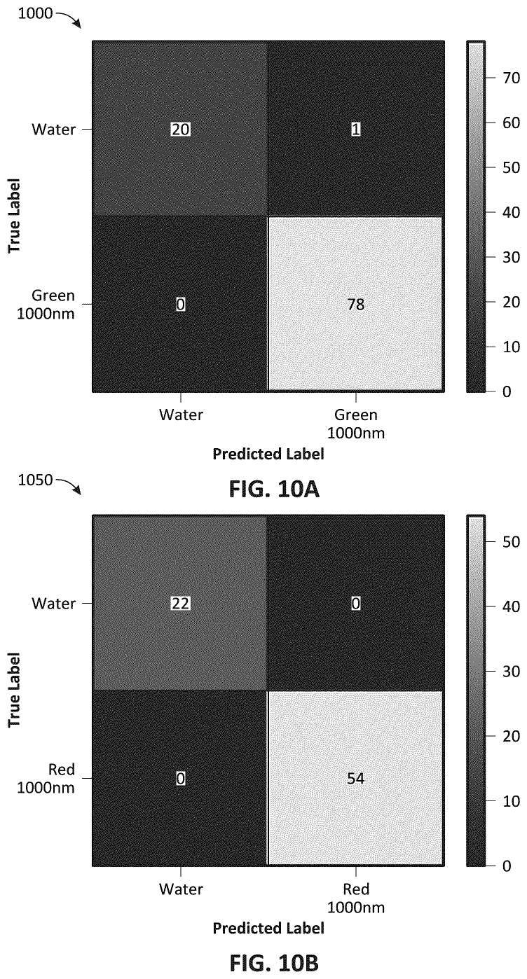

FIGS. 10A and 10B depict a confusion matrix 1000 and a confusion matrix 1050, respectively, for results of applications of two sets of trained decision trees to multiple sets of values based on intensity measurements of multiple instances of light that has passed through samples containing 1000 nanometer microspheres or samples that do not contain microspheres in some embodiments. The set of trained decision trees that produced the results in the confusion matrix 1000 was trained on samples containing 1000 nanometer green microspheres at ten different concentrations, operated in a binary mode, and there were 99 total samples, including samples containing microspheres in water (for example, deionized water) and samples containing only water (for example, deionized water). The set of trained decision trees that produced the results in the confusion matrix 1050 was trained on samples containing 1000 nanometer red microspheres at ten different concentrations, operated in a binary mode, and there were 76 total samples, including samples containing microspheres in water (for example, deionized water) and samples containing only water (for example, deionized water).

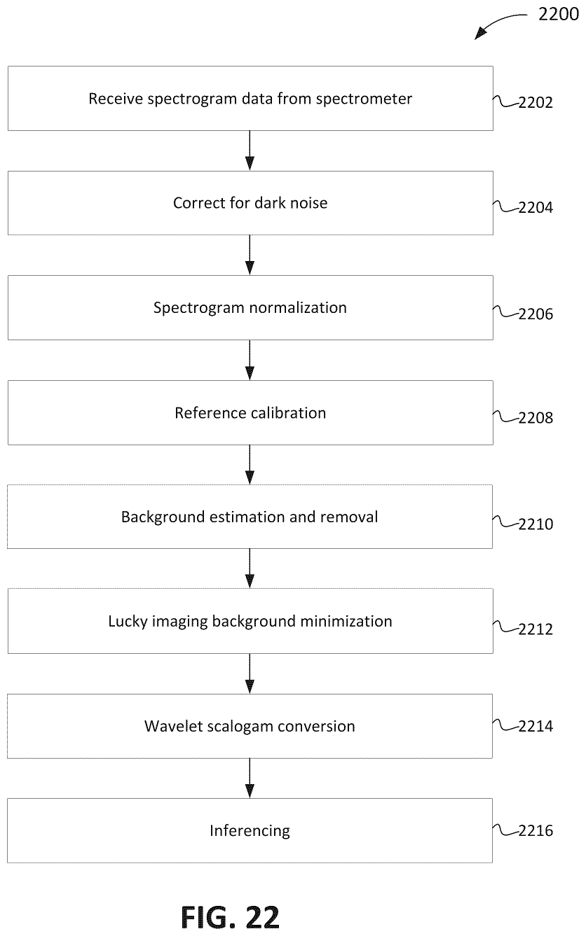

In both the confusion matrix 1000 and the confusion matrix 1050, the upper left hand quadrant indicates true negatives, of which there are 20 (FIG. 10A) or 22 (FIG. 10B). The upper right hand quadrant indicates false negatives, of which there are 1 (FIG. 10A) or 0 (FIG. 10B). The lower left hand quadrant indicates false positives, of which there are 0 (both FIG. 10A and FIG. 10B). The lower right hand quadrant indicates true positives, of which there are 78 (FIG. 10A) or 54 (FIG. 10B). The confusion matrix 1000 indicates that the set of trained decision trees is 99% accurate at detecting the presence of 1000 nanometer green microspheres in concentrations as low as 1 microsphere/mL and the confusion matrix 1050 indicates that the set of trained decision trees is 100% accurate at detecting the presence of 1000 nanometer red microspheres in concentrations as low as 1 microsphere/mL. 1000 nanometer microspheres are approximately the same size as common bacteria such as salmonella and E. coli.

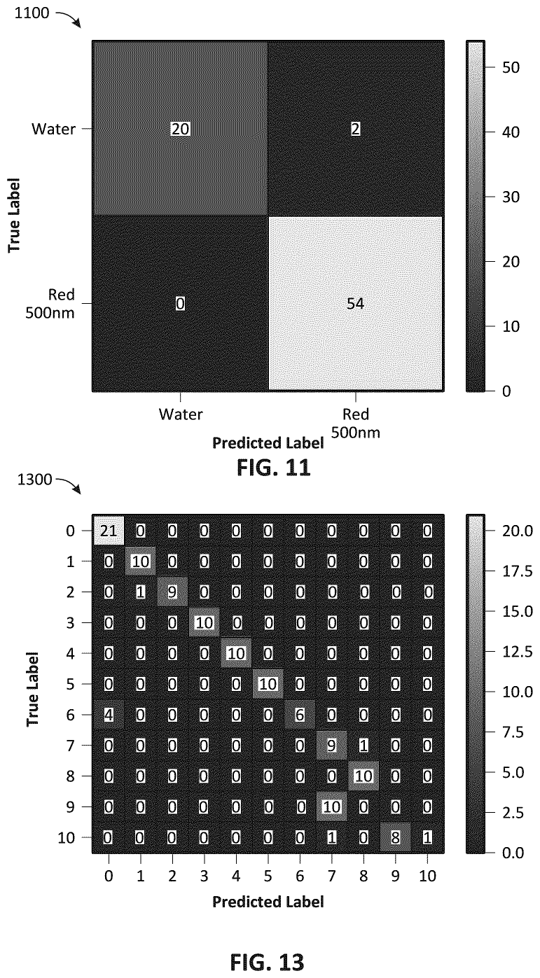

FIG. 11 depicts a confusion matrix 1100 for results of applications of a set of trained decision trees to multiple sets of values based on intensity measurements of multiple instances of light that has passed through samples containing 500 nanometer microspheres or samples that do not contain microspheres in some embodiments. The set of trained decision trees that produced the results in the confusion matrix 1100 was trained on samples containing 500 nanometer red microspheres at ten different concentrations, operated in a binary mode, and there were 76 total testing samples, including testing samples containing microspheres in water (for example, deionized water) and testing samples containing only water (for example, deionized water). The upper left hand quadrant of the confusion matrix 1100 indicates true negatives, of which there are 20. The upper right hand quadrant of the confusion matrix 1100 indicates false negatives, of which there are 2. The lower left hand quadrant of the confusion matrix 1100 indicates false positives, of which there are 0. The lower right hand quadrant of the confusion matrix 1100 indicates true positives, of which there are 54. The confusion matrix 1100 indicates that the set of trained decision trees is 97% accurate at detecting the presence of 500 nanometer red microspheres in concentrations as low as 1e² microspheres/mL.

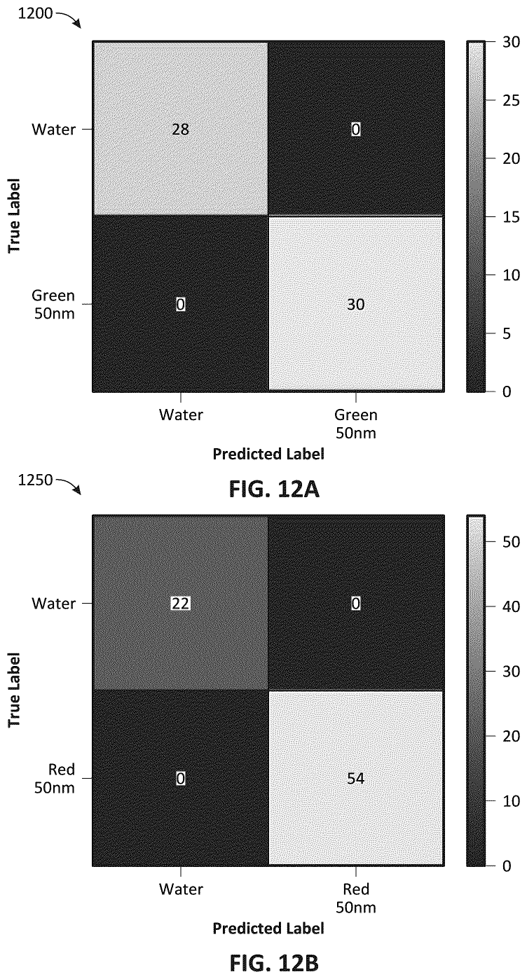

FIGS. 12A and 12B depict a confusion matrix 1200 and a confusion matrix 1250, respectively, for results of applications of two sets of trained decision trees to multiple sets of values based on intensity measurements of multiple instances of light that has passed through samples containing 50 nanometer microspheres or samples that do not contain microspheres in some embodiments. The set of trained decision trees that produced the results in the confusion matrix 1200 was trained on samples containing 50 nanometer green microspheres at ten different concentrations, operated in a binary mode, and there were 58 total testing samples, including testing samples containing microspheres in water (for example, deionized water) and testing samples containing only water (for example, deionized water). The set of trained decision trees that produced the results in the confusion matrix 1250 was trained on samples containing 50 nanometer red microspheres at ten different concentrations, operated in a binary mode, and there were 76 total testing samples, including testing samples containing microspheres in water (for example, deionized water) and testing samples containing only water (for example, deionized water). In both the confusion matrix 1200 and the confusion matrix 1250, the upper left hand quadrant indicates true negatives, of which there are 28 (FIG. 12A) or 22 (FIG. 10B). The upper right hand quadrant indicates false negatives, of which there are 0 (both FIG. 12A and FIG. 2B). The lower left hand quadrant indicates false positives, of which there are 0 (both FIG. 12A and FIG. 12B). The lower right hand quadrant indicates true positives, of which there are 30 (FIG. 12A) or 54 (FIG. 12B). The confusion matrix 1200 indicates that the set of trained decision trees is 100% accurate at detecting the presence of 50 nanometer green microspheres in concentrations as low as les microspheres/mL and the confusion matrix 1250 indicates that the set of trained decision trees is 100% accurate at detecting the presence of 1000 nanometer red microspheres in concentrations as low as 1e⁵ microspheres/mL.

FIG. 13 depicts a confusion matrix 1300 for results of applications of a set of trained decision trees to multiple sets of values based on intensity measurements of multiple instances of light that has passed through samples containing green microspheres at ten different concentrations or samples that do not contain microspheres in some embodiments. The set of trained decision trees that produced the results in the confusion matrix 1300 operated in a multiclass mode and there were 121 total testing samples. In the set of testing samples, there were different testing samples containing ten different concentrations of green microspheres and testing samples that did not contain microspheres. The confusion matrix 1300 indicates that the set of trained decision trees operating in the multiclass mode accurately identifies the concentration of a testing sample up to the fifth concentration (for example, a concentration at approximately 1e⁶ microspheres/mL). The confusion matrix 1300 further indicates that the set of trained decision trees operating in the multiclass mode accurately identifies an approximate range of concentrations for the sixth concentration through the tenth concentration.

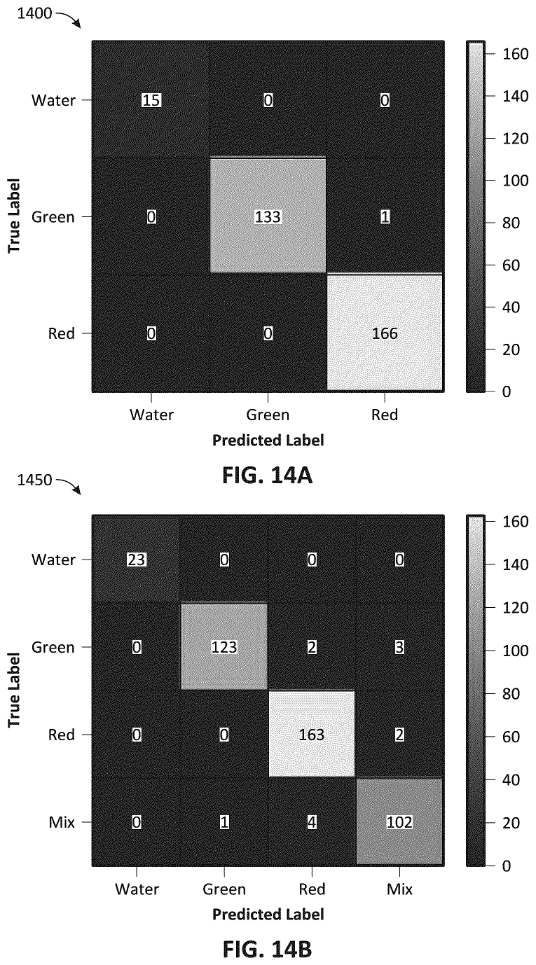

FIG. 14A depicts a confusion matrix 1400 for results of applications of a set of trained decision trees to multiple sets of values based on intensity measurements of multiple instances of light that has passed through samples containing green microspheres, samples containing red microspheres, or samples that do not contain microspheres in some embodiments. The set of trained decision trees that produced the results in the confusion matrix 1400 operated in a multiclass mode and there were 315 total testing samples, including testing samples containing microspheres in water (for example, deionized water) and testing samples containing only water (for example, deionized water). The testing samples contained red or green microspheres of different sizes and at different concentrations. The confusion matrix 1400 indicates that the set of trained decision trees is 99% accurate at detecting the presence and color of microspheres as small as 50 nm in concentrations as low as 1 microsphere/mL.

FIG. 14B depicts a confusion matrix 1450 for results of applications of a set of trained decision trees to multiple sets of values based on intensity measurements of multiple instances of light that has passed through samples containing green microspheres, samples containing red microspheres, samples containing a mixture of red microspheres and green microspheres, or samples that do not contain microspheres in some embodiments. The set of trained decision trees that produced the results in the confusion matrix 1450 operated in a multiclass mode and there were 423 total testing samples, including testing samples containing microspheres in water (for example, deionized water) and testing samples containing only water (for example, deionized water). The testing samples contained red or green microspheres of different sizes and at different concentrations. The confusion matrix 1400 indicates that the set of trained decision trees is 97% accurate at detecting the presence, color, and mixture of microspheres as small as 50 nm in concentrations as low as 1 microsphere/mL.

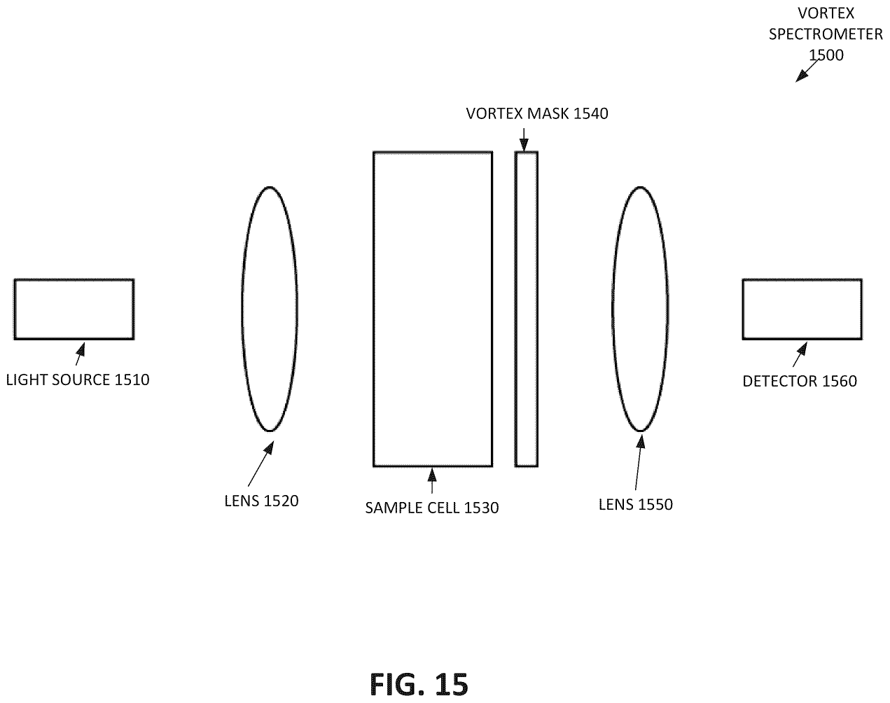

FIG. 15 depicts an example vortex spectrometer 1500 in some embodiments. The vortex spectrometer 1500 depicted in FIG. 15 is simplified. It may be appreciated that the vortex spectrometer 1500 may include an aperture for controlling wavelengths, filters, beam splitters, diffraction grating, and the like as discussed herein. The vortex spectrometer 1500 and other spectrometers described herein may be utilized to detect pathogens, including common foodborne pathogens such as E. coli and salmonella and other pathogens such as a SARS-CoV-2 virion, which has an average size of 95 nm. The vortex spectrometer 1500 and other spectrometers described herein may be described as detecting pathogens such as the SARS-CoV-2 virion. Furthermore, models may be described herein as being used to determine infection of a person or non-infection of a person based on the detection or non-detection of such pathogens by the vortex spectrometer 1500 and other spectrometers. The vortex spectrometer 1500 and other spectrometers described herein may also be utilized to detect common foodborne pathogens such as E. coli and salmonella. Furthermore, models may be described herein as being used to determine contamination or non-contamination of food processing byproducts based on the detection or non-detection or such pathogens by the vortex spectrometer 1500 and other spectrometers described herein. Accordingly, although the discussion herein may refer to the presence or absence of pathogens such as the SARS-CoV-2 virion and the determination of infection or non-infection of a person, the discussion herein is also applicable to the presence or absence of foodborne pathogens such as E. coli and salmonella and the determination of contamination or non-contamination of food processing byproducts and other items.

The hardware of the vortex spectrometer 1500 may be responsible for shaping and directing light through the collected sample, resolving the light by wavelength, and measuring the intensity. In various embodiments, light source (e.g., a standard tungsten-halogen source) emits broad bandwidth light spanning from the ultraviolet to near-infrared ranges which passes through the sample and cuvette. The vortex spectrometer 1500 may measure the amount of absorption, scattering and fluorescence of wavelengths of light from the ultraviolet to the near infrared by the sample, this combination of phenomena is collectively known as the spectral features of the sample. The vortex spectrometer 1500 may count the arrival of particles of light on a sensor to produce the transmission spectrum. High reliability for the optical system may be achieved by using optical fiber coupling of the major components, with free space propagation of light through the sample, cuvette, and collimation and refocusing lenses.

The vortex spectrometer 1500 of FIG. 15 includes a light source 1510 (e.g., standard tungsten-halogen source or laser), first lens 1520, sample cell 1530, vortex mask 1540, a second lens 1550, and a detector 1560. Light from a light source 1510 may be collimated by the first lens 1520. The collimated light passes through a sample cell (e.g., containing a sample of a food processing byproduct), a vortex mask 1540, and the second lens 1550 before passing to the detector 1560.

When the light passes through a scattering medium containing particles larger than the wavelength, light is scattered. The most intense scattering usually occurs in the forward direction. Light scattered along the optical axis is often difficult to distinguish from the superimposed unscattered laser beam, especially when there is a dilute concentration of weak scatterers. This scattered light may interfere with the light of the principal beam and, as a result, speckle (i.e., noise) may be formed.

In some cases, particular wavelengths may be absorbed by the scattering media. This occurs because the light at those particular wavelengths excite the rotational or vibrational state of the molecules in the media. Therefore, the chemical makeup of an absorbing media may be based on the spectral absorption signature that is present. If the medium is weakly scattering (i.e., there are few scatterers), the absorption signature may be overwhelmed by the strong on-axis unscattered light source. Therefore, in order to optimize the characterization of the scattering molecules, a light suppression technique may be utilized to attenuate the strong on-axis source while leaving the weaker scattered signal intact.

The vortex mask 1540 is a dark null of destructive interference that occurs at a spiral phase dislocation in a beam of spatially coherent light. The phase of a transmitted light beam may be twisted and light from opposite sides of the mask may coherently destructively interfere to form a dark null in the transmitted intensity pattern, much like the eye of a hurricane.

The vortex mask 1540 may assist in creating destructive interference of the light source, thereby enabling improved sensitivity of fainter signals. In one example of the optical path shown in FIG. 15, light is projected from the light source 1510 through the sample cell 1530. The light then passes through the vortex mask 1540 to be detected by the detector 1560 which may digitize the signal as a function of wavelength and provides the signal for further analysis and/or display.

In some embodiments, divergent light may be collimated by a concave mirror and directed into a grating to disperse the spectral components of the light at slightly varying angles which may be focused by a second concave mirror and imaged onto a detector.

The vortex mask 1540 may be a vortex coronagraph configured to reduce unwanted glare from a spectrometer light source. As discussed herein, the spectrometer may include or be coupled to a vortex mask in order to reduce or eliminate undesired wavelengths and/or light intensities of the light that passed through the sample cell 1530. The vortex mask 1540 may include or be an optical vortex coronagraph that uses a phase-mask in which the phase-shift varies azimuthally around the center. The vortex mask 1540 may use interference to mask out light along the center axis of the optical path of the spectrometer but allows light from off axis.

A vortex mask 1540 may be used to create an optical vortex to reduce or eliminate unwanted light from the spectrometer light sources. Without reducing undesirable light from the spectrometer light sources, many signals may otherwise be too faint to be detected (e.g., faint signals from desired absorption or transmittance is overwhelmed by the other signals caused by the light sources).

In some embodiments, the vortex mask 1540 may be or utilize an optical vortex coronagraph. An example optical vortex coronagraph uses a helical phase of the form eiϕ, with ϕ=lθ, where l is the topological charge and θ is the focal plane azimuthal coordinate. In optical systems, vortices manifest themselves as dark donut of destructive interference that occur at phase singularities. For example, E(ρ, ϕ, z, t)=A(ρ, z) exp(ilθ) exp(iωt−ikz) where (ρ, ϕ, z) are cylindrical coordinates, A(ρ, z) is a circularly symmetric amplitude function and k=2π/λ, is the wavenumber of a monochromatic field of wavelength k.

In some embodiments, the optical vortex coronagraph may utilize a rotationally symmetric half wave plate which can generate an azimuthal phase spiral reaching an even multiple of 2 pi radian.

The vortex mask 1540 may include an optical vortex induced by an achromatic subwavelength grating. In some embodiments, the vortex mask 1540 may be an annular groove phase mask coronagraph. As discussed herein, without the vortex mask 1540, detection of faint sources around significant noise may be difficult due to the large ratio between them.

In various embodiments, the vortex mask 1540 is not a pure amplitude mask, a pure phase mask, a single pupil achromatic nulling interferometer, or a monochromatic pupil plane mask. In one example, the vortex mask 1540 may be an annular groove phase mask coronagraph. The vortex mask 1540 may include a focal plane that is divided into four equal areas centered on an optical axis. Unlike a mask where two of the focal planes are on a diagonal providing a π phase shift to cause destructive interference inside a geometric pupil area, the vortex mask 1540 utilizes subwavelength gratings while suppressing “dead zones” (e.g., where potential circumstellar signal or companion is attenuated by up to 4 magnitudes). The vortex mask 1540 may include concentric circular subwavelength gratings.

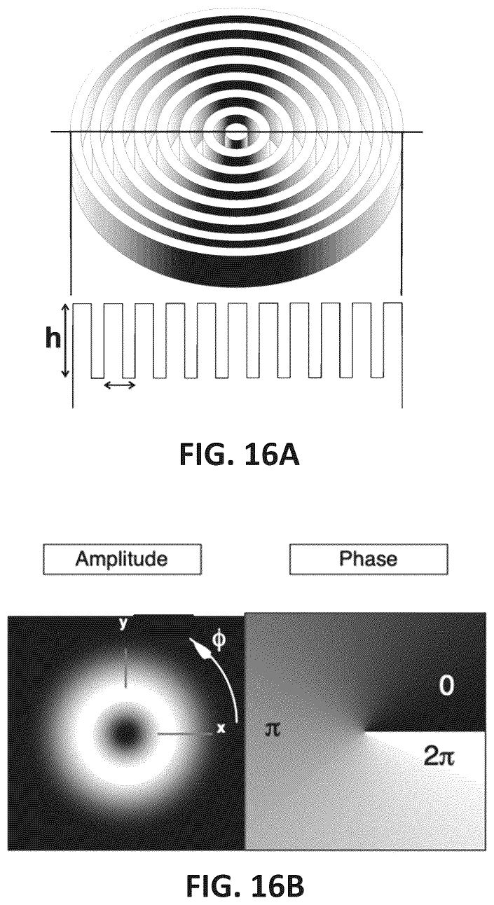

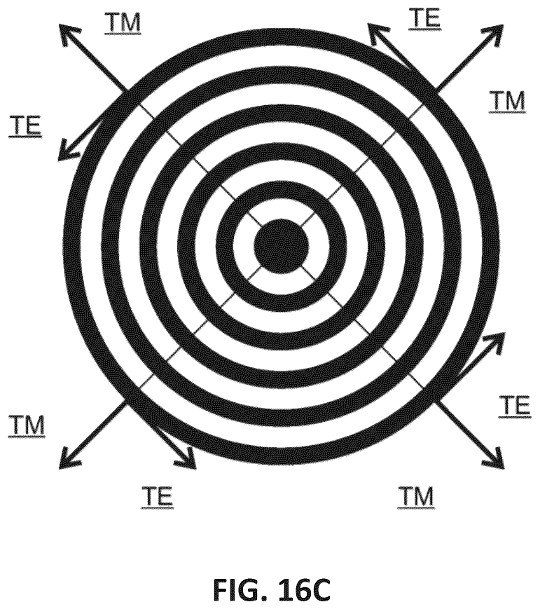

The vortex mask 1540 may include a focal plane micro-component including a concentric circular surface-relief grating with rectangular grooves of depth h and equally separated by a period A. FIG. 16A depicts an example coronagraph scheme including a concentric circular surface relief grating with rectangular grooves with depth h and a periodicity of A. in some embodiments, the vortex mask 1540 may be a vectorial phase mask (i.e., the vortex mask 1540 induces a differential phase shift between the local polarization states of the incident natural (or polarized) light).

When the period A of the grating is smaller than the wavelength of the incident light, the vortex mask 1540 does not diffract as a classical spectroscopic grating. Incident energy is enforced to propagate only in the zeroth order, leaving incident wavefronts free from any further aberrations. In various embodiments, the subwavelength gratings of the vortex mask 1540 may be Zeroth Order Gratings.

By controlling the geometry of the grating structure, the vortex mask 1540 may be tuned (e.g., to make the form birefringence proportional to the wavelength in order to achromatize the subsequent differential 7L phase shift between two polarization states). This may create an optical vortex where phases possess a screw dislocation inducing a phase singularity. The central singularity forces the intensity to vanish by a total destructive interference, creating a dark core. This dark core propagates and is conserved along the optical axis. In various embodiments, the vortex mask 1540 creates an optical vortex in the focal plane, filtering in the relayed pupil plane and making the detection in a final image plane. FIG. 16B includes images of amplitude and phase caused by the vortex mask 1540 in some embodiments.

In various embodiments, the vortex mask 1540 may be fabricated by imprinting the concentric annular mask in a resin coated on a chosen substrate material. For example, fabrication may be performed, in part, by laser direct writing or e-beam lithography. This process may define the lateral dimensions of the Zeroth Order Gratings (ZOG). This pattern may then be uniformly transferred in the substrate by an appropriate reactive plasma ion beam etching down to the desired depth.

In some embodiments, a space-variant half-wave plate may be used to generate the optical vortex. In one example, a beam of light containing an optical vortex is described by an electric field distribution that may be expressed E(x, y, z)=A(x, y, z)exp(iΦ(x, y, z))exp(imθ) where A and ϕ are arbitrary amplitude and phase functions respectively. θ is an angle about the vortex core located at (xᵥ, yᵥ): x−xᵥ=cos θ and y−yᵥ=sin θ, and m is an integer called the vortex charge (or vortex topological charge). There are various techniques to convert a given input beam into an output containing an arbitrary distribution of optical vortices. In this example, this method makes use of a space variant half-wave retarder and a circularly polarized input beam. For convenience, the input beam is right circularly polarized.

A conventional half-wave plate may convert a right circularly polarized beam into a left circularly polarized beam without introducing a spatially varying phase on the output beam. This may be accomplished with a birefringent material such as a nematic liquid crystal. In this example, the refractive index depends on the linear polarization components of the beam. The horizontal and vertical polarization components of the right circularly polarized input beam may be represented by variable E_{x,in}=1 and E_{y,in}=−i, where i=\sqrt{−1}. The output beam may have horizontal and vertical components that are a linear combination of the input components. For a half-wave retarder with the fast crystal axis making an angle θ with respect to the x-axis, the output field may be expressed:

$$[ \begin{matrix}{E}_{x , out} \\ {E}_{y , out}\end{matrix} ] = [ \begin{matrix}cos {θ}^{′} & - sin {θ}^{′} \\ sin {θ}^{′} & cos {θ}^{′}\end{matrix} ] [ \begin{matrix}exp ( i π / 2 ) & 0 \\ 0 & exp ( - i π / 2 )\end{matrix} ] [ \begin{matrix}cos {θ}^{′} & sin {θ}^{′} \\ - sin {θ}^{′} & cos {θ}^{′}\end{matrix} ] [ \begin{matrix}{E}_{x , in} \\ {E}_{y , in}\end{matrix} ]$$

when

$${θ}^{′} = \frac{π}{4} ,$$

E_{x,out}=1 and E_{y,out}=i, which describes left circular polarization. The principle of a space-variant half-wave retarder can be reduced making use of the trigonometric identity tan(2u)=2 tan u/(1−tan² u)

$$[ \begin{matrix}{E}_{x , out} \\ {E}_{y , out}\end{matrix} ] = {ie}^{- i ϕ} [ \begin{matrix}1 \\ i\end{matrix} ]$$

where tan ϕ=tan 2θ′, or equivalently, ϕ=2θ′. The spatial phase distribution of the output left circularly polarized beam may be controlled by spatially varying the angle of the crystal fast axis. For example, for a vortex of charge m=−2 having a spatial phase distribution exp(−i2θ), the fast axis of the crystal may be spatially oriented by the exact angular coordinate θ′=θ, where θ corresponds to the (x,y) location of the material: x=cos θ, y=sin θ. Likewise, for a vortex beam of charge m=−4, the fast axis is rotated by an amount θ′=2θ.

The half-wave phase factors in the equation above, exp(±iπ/2 may be achieved when the following birefringent material condition is satisfied: π(nₑ−nₒ)L/λ=λ/2 where nₒ and nₑ are the ordinary and extraordinary refractive indexes, respectively, L is the thickness of the material, and λ is the wavelength of light. This “half-wave” condition can only be satisfied at a single wavelength. The conversion efficiency of the right circularly polarized input beam to the left circularly polarized output beam having a vortex phase decreases as a function of wavelength. To rectify this shortcoming and for efficiency across a band of wavelength, an achromatic half-wave retarder may be used.

Broadband wave retarders may be constructed by stacking multiple layers of the same birefringent material at different orientations. Achromatic and superachromatic wave plates may be constructed from three more layers. A three-layer achromatic half-wave plate is described below. The electric field vector may be described with Jones matrix formalism:

$$[ \text{} \begin{matrix}{E}_{x , out} \\ {E}_{y , out}\end{matrix} ] = [ \begin{matrix}{C}_{1 , 1} & {C}_{1 , 2} \\ {C}_{2 , 1} & {C}_{2 , 2}\end{matrix} ] [ \text{} \begin{matrix}{B}_{1 , 1} & {B}_{1 , 2} \\ {B}_{2 , 1} & {B}_{2 , 2}\end{matrix} ] [ \text{} \begin{matrix}{A}_{1 , 1} & {A}_{1 , 2} \\ {A}_{2 , 1} & {A}_{2 , 2}\end{matrix} ] [ \text{} \begin{matrix}{E}_{x , in} \\ {E}_{y , in}\end{matrix} ] = [ \text{} \begin{matrix}{M}_{1 , 1} & {M}_{1 , 2} \\ {M}_{2 , 1} & {M}_{2 , 2}\end{matrix} ] [ \text{} \begin{matrix}{E}_{x , in} \\ {E}_{y , in}\end{matrix} ]$$

$$where$$

$$[ \begin{matrix}{A}_{1 , 1} & {A}_{1 , 2} \\ {A}_{2 , 1} & {A}_{2 , 2}\end{matrix} ] = [ \begin{matrix}cos {θ}_{a} & - sin {θ}_{a} \\ sin {θ}_{a} & cos {θ}_{a}\end{matrix} ] [ \begin{matrix}exp ( i {γ}_{a} ) & 0 \\ 0 & exp ( - i {γ}_{a} )\end{matrix} ] [ \begin{matrix}cos {θ}_{a} & sin {θ}_{a} \\ - sin {θ}_{a} & cos {θ}_{a}\end{matrix} ]$$

$$[ \begin{matrix}{B}_{1 , 1} & {B}_{1 , 2} \\ {B}_{2 , 1} & {B}_{2 , 2}\end{matrix} ] = [ \begin{matrix}cos {θ}_{b} & - sin {θ}_{b} \\ sin {θ}_{b} & cos {θ}_{b}\end{matrix} ] [ \begin{matrix}exp ( i {γ}_{b} ) & 0 \\ 0 & exp ( - i {γ}_{b} )\end{matrix} ] [ \begin{matrix}cos {θ}_{b} & sin {θ}_{b} \\ - sin {θ}_{b} & cos {θ}_{b}\end{matrix} ]$$

$$[ \begin{matrix}{C}_{1 , 1} & {C}_{1 , 2} \\ {C}_{2 , 1} & {C}_{2 , 2}\end{matrix} ] = [ \begin{matrix}cos {θ}_{c} & - sin {θ}_{c} \\ sin {θ}_{c} & cos {θ}_{c}\end{matrix} ] [ \begin{matrix}exp ( i {γ}_{c} ) & 0 \\ 0 & exp ( - i {γ}_{c} )\end{matrix} ] [ \begin{matrix}cos {θ}_{c} & sin {θ}_{c} \\ - sin {θ}_{c} & cos {θ}_{c}\end{matrix} ]$$

Although the ordinary nₒ and the extraordinary nₑ refractive indexes vary with wavelength, for first order design purposes the birefringence Δn=nₑ−nₒ is often assumed to be nearly constant. The wavelength-dependent phase retardance [symbol] or a layer of thickness L may be expressed: 2γ=ΔΦ(λ)=2π(nₑ−nₒ)L/λ≈2πLΔn(1−δλ/λ)/λ₀ where λ=λ₀+δλ and λ₀ is a central design wavelength for the achromatic retarder.

The waveplate may be achromatized if γₐ=γ_{c} and θₐ=θ_{c}. In effect, the first and last materials may be the same and the orientations are parallel. The final conditions are that cos 2θ_{b}=−γ_{b,0}/2γ_{a,0} and γ_{b,0}=π/2. Hence cos 2θ_{b}=−π/4γ_{a,0}.

FIG. 16C depicts an example of a vortex mask which can be seen as a polarization FQ-PM. The parallel potentially interfering polarization states are out of phase according to the FQ-PM focal plane phase shift distribution. ϕTE and ϕTM are the output phases of the polarization components TE and TM such that ΔϕTE−TM=|ϕTE−ϕTM|=π. While some constructions and configurations of AGPMs have been used for astronomy, none have been used for spectroscopy for detection of information in faint signals with significant noise.

The vortex mask 1540 may be complemented by a diaphragm in the relayed pupil plane (“lyot stop”) to suppress diffracted light.

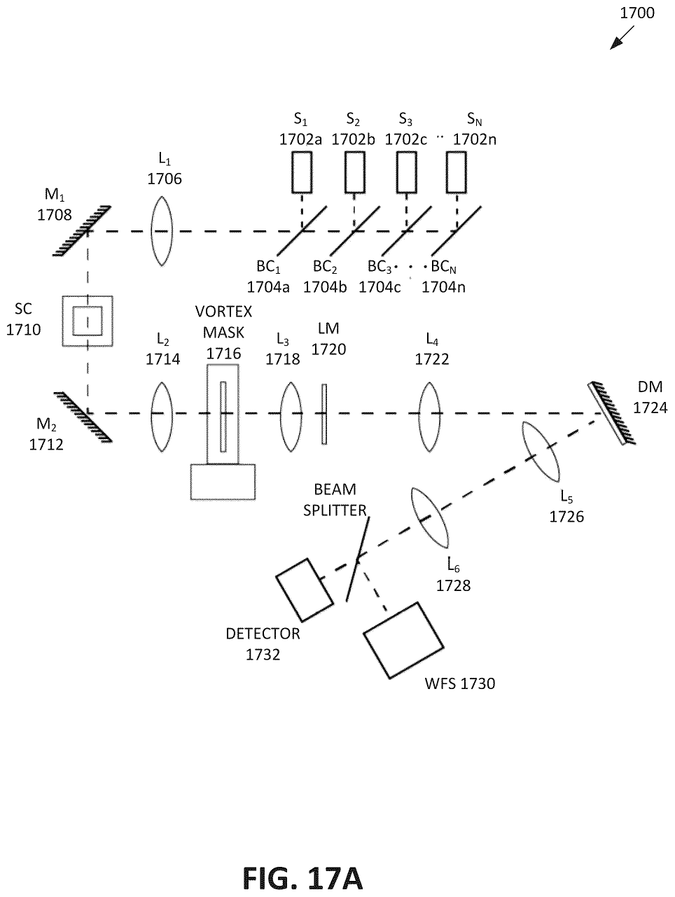

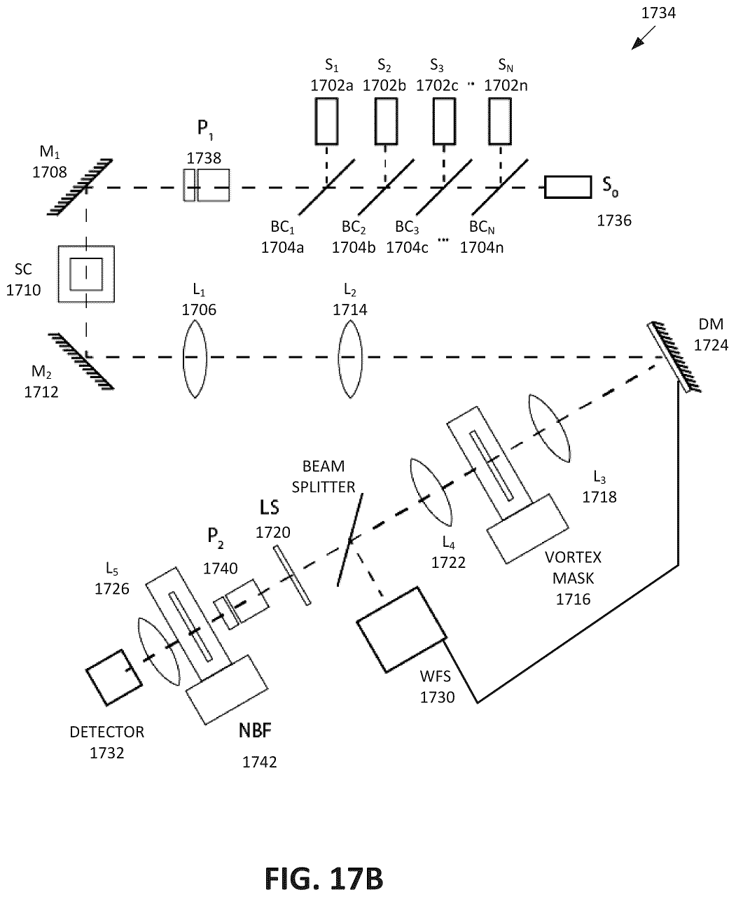

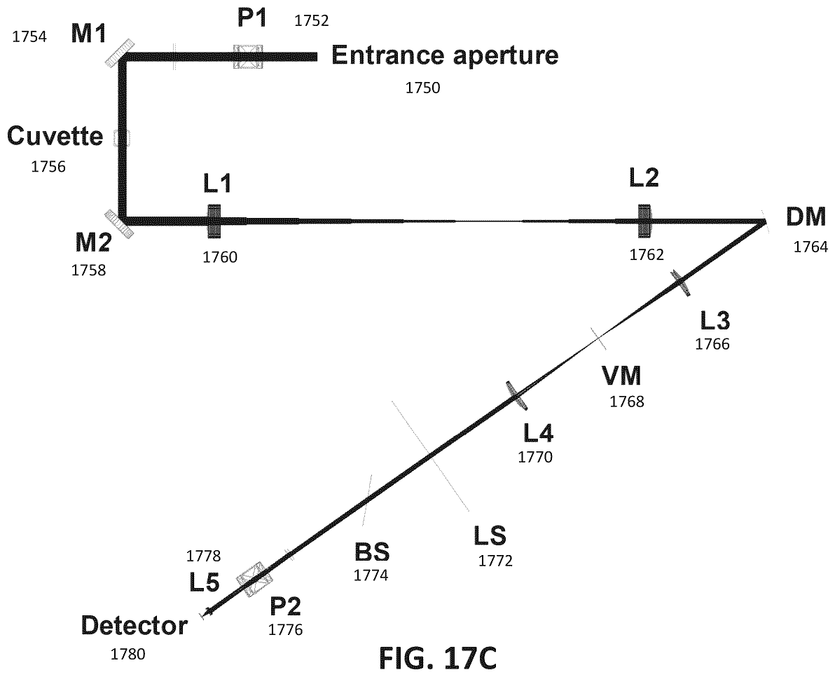

FIG. 17A depicts an example simplified spectrometer optical path 1700 in some embodiments. One or more light sources may project desired wavelengths along the optical path 1700 through the sample chamber 1710 and then through a vortex mask 1716 to a detector 1732. The vortex mask 1716 may assist with improved signal measurement and signal boosting. As such, measurements of the resulting signal enable a discriminator to detect viruses and/or substances related to viruses (e.g., proteins) to detect infections that were previously too faint to detect.

In various embodiments, the optical path 1700 includes a vortex mask 1716 but not a lyot mask 1720. In other embodiments, the optical path 1700 includes a vortex mask and a lyot mask.

Light sources 1702a-n each project light at a different wavelength. In some embodiments, a single laser projects coherent light through a differential grating to separate the wavelengths. In other embodiments, different light sources may project different wavelengths (1702a may be a different wavelength from 1704b and the like). Each Sn may be a different and distinct wavelength as compared to all other sources.

The light sources 1702a-n may be or include five co-bore-sighted laser sources that create a light source with an 8 mm collimated beam (or another diameter beam may be produced such as 3 mm, 4 mm, 5 mm, 6 mm, 7 mm, 9 mm, or 10 mm for example). Each light source 1702a-n may be or include an FC fiber connected to an achromatic collimator that sets the output beam width. In one example, light sources 1702a-n are diode laser sources of various wavelengths. Collimated light from each light source 1702a-n is reflected from the surface of a 55/45 beam splitter or beam comber (BC1-BC4).

Beam combiners 1704a-1704n each may allow some wavelengths to pass while reflecting at least one wavelength (e.g., combining optical wavelengths). In one example, beam combiner 1704a may reflect light at a first wavelength from source 1702a and the beam combiner 1704a may allow other wavelengths to pass through (e.g., light from sources 1702b-1702n). The light from each source may be projected through lens 1706. Lens 1706 may be a collimator to collimate the light received from the light sources.

Reflective surface 1708 and reflective surface 1712 may reflect all light from the sources. In one example, light from sources 1702a-1702n is reflected by reflective surface 1708 through sample chamber 1710. The sample chamber 1710 may contain a of a food processing byproduct from the food processing apparatus 106. In various embodiments, the sample chamber 1710 is or contains the cuvette. In another example, the sample chamber 1710 is or contains transparent substrates. The light from the sources pass through the sample chamber 1710 and then is reflected by reflective surface 1712.

The second section of the optical path 1700 propagates the collimated beam through a scattering sample of the sample chamber 1710. In one example, a collimated beam from the light source is reflected perpendicularly from reflective surface 1708 through a sample cuvette holder (i.e., the sample chamber 1710). In this example, the entrance aperture of the sample chamber 1710 has a 9 mm diameter. The sample chamber 1710 may contain a sample in a liquid medium and may have a width of 10 mm perpendicular to the beam and a length parallel to the beam of 2 mm.

In one example, the sample chamber 1710 may be filled with approximately 1 ml of liquid so the full 8 mm beam passes through the sample. The residual collimated beam and the light scattered off the sample may then reflect perpendicularly off of reflective surface M2 1712 and exit to the next section of the optical path.

Light then is further focused by lens 1714 on the vortex mask 1716. The lens 1718 may focus the light on the lyot mask 1720, which may be optional, and/or may collimate the light received from the vortex mask 1716.

The lyot mask 1720, which may be optional, may be a lyot-mask (e.g., lyot stop) such as a lyot-plane phase mask, which enables improved contrast performance. The lyot-plane phase mask may relocate residual light away from a region of the image plane, thereby reducing light noise from the sources of the spectrometer and improves sensitivity to off-axis scattered light.

It may be appreciated that, in some embodiments, the spectrometer includes a vortex mask 1716, a lyot mask 1720, or both (e.g., the spectrometer may include a lyot mask 1720 but not a vortex mask 1716, a lyot mask 1720 and a vortex mask 1716, or vortex mask 1716 but not a lyot mask 1720).

The lens 1722 may collimate the light and/or focus on the light on the optional deformable mirror 1724. In some embodiments, the lens 1722 may focus the light on the deformable mirror 1724 (e.g., to a desired diameter).

The deformable mirror 1724 may, in some embodiment, may control the wave front of the light based on information received from the wavefront sensor 1730. In this example, the light may magnify and/or enhance the light of the optical path. Control of the deformable mirror 1724 may allow for control of the wave front of the light to direct a flat wave front to the detector 1732. It will be appreciated that, in some examples, the optical path 1700 may not have a deformable mirror 1724. In that case, the optical path 1700 may not have a beam splitter or a wave front sensor 1730.

The detector 1732 detects spectral components (e.g., intensities of received wavelengths). In various embodiments, the detector 1732 is part of a spectrometer, a photodiode, or an LCD camera. The detector may generate measurements indicating intensities of wavelengths from the incoherent light of the optical path. The detector may provide absorption or transmittance measurements related to the particles and components of the sample.

In one example, the detector 1732 is in communication with a processor to assess and generate the measurement results. The measurement results may then be used to identify if the sample contains a pathogen.

The measurement results may be received by a discriminator. A discriminator may categorize or determine if the patron is infected by assessing and/or analyzing the measurement results. The discriminator may assess the measurement results using a logistic regression technique, an AI approach (e.g., convolutional neural network), and/or other statistical methods. In some embodiments, the measurement results may be used to create and/or train the discriminator.

In various embodiments, there is a beam splitter in the optical path before the detector thereby enabling the beam to be split between the detector 1732 and the wavefront sensor 1730. A wavefront sensor 1730 is a device for measuring aberrations in an optical wavefront (e.g., points where the wave has the same phase as the sinusoid) and controlling the deformable mirror 1724 to correct and flatten the optical wavefront.

Lens 15 1726 and lens 16 1728 may also focus and collimate the light to project to the wavefront sensor 1730 and/or detector 1732.

FIG. 17B depicts another example simplified spectrometer optical path 1734 in some embodiments. Similar to FIG. 17A, the light sources 1736 and 1702a-n project desired wavelengths along the optical path 1734 through the sample chamber 1710 and then through a vortex mask 1716 to a detector 1732. The vortex mask 1716 may assist with improved signal measurement and signal boosting. As such, measurements of the resulting signal enable a discriminator to detect viruses and/or substances related to viruses (e.g., proteins) to detect infections that were previously too faint to detect. In this example, different from FIG. 17A, the vortex mask 1716 and the lyot mask 1720, which may be optional, has been moved to after the deformable mirror 1724.

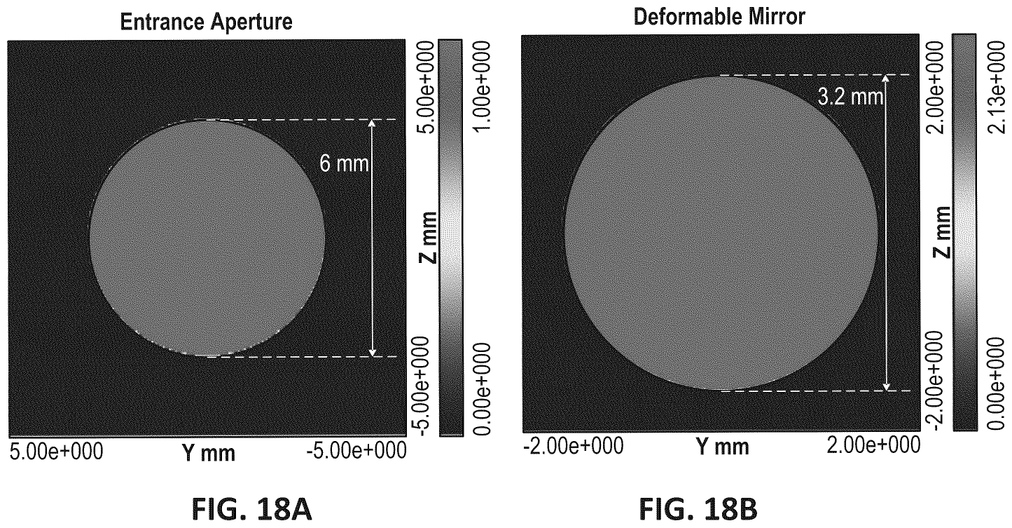

In various embodiments (e.g., in any spectrometer discussed herein), the beam size may be narrowed to ensure that the beam passes through the cuvette and does not clip a corner or edge of the cuvette. The beam size may be 4 mm from the light source (e.g., at the entrance aperture) for example. Other examples of the beam size may be 4 mm to 8 mm. The lens from M2 1712 may be reduced to 3.2 mm on the deformable mirror 1724. Other examples of the beam size may be 3 mm to 4 mm. In some embodiments, lens 1706 and 1714 reduce the beam to the deformable mirror 1724. FIG. 18A depicts a measurement of the aperture of an entrance aperture as being 6 mm in one example. In this example, the aperture accommodates an optical beam with a 6 mm diameter. FIG. 18B depicts a measurement of an optical beam received and reflected by a deformable mirror in some embodiments. In this example, the deformable mirror accommodates an optical beam of a 3.2 mm diameter received from one or more lenses along the optical path 1734.

In various embodiments, the optical path 1734 includes a vortex mask 1716 but not a lyot mask 1720. In other embodiments, the optical path 1734 includes a vortex mask 1716 and a lyot mask 1720.

Light sources 1736 and 1702a-n each project light at a different wavelength. In some embodiments, a laser projects coherent light through a differential grating to separate the wavelengths. In other embodiments, different light sources may project different wavelengths (1702a may be a different wavelength from 1704b and the like). Each Sn may be a different and distinct wavelength as compared to all other sources.

The light sources 1736 and 1702a-n may be or include five co-bore-sighted laser sources that create a light source with an 8 mm collimated beam. The light source S0 1736 may be a control wavelength. In some embodiments, the light source S0 1736 is 635 nm.

The light sources 1702a-n and/or the light source 1736 may be or include five co-bore-sighted laser sources that create a light source with an 8 mm collimated beam. Each light source 1736 and 1702a-n may be or include an FC fiber connected to an achromatic collimator that sets the output beam width to 8 mm. In one example, light sources 1736 and 1702a-n are diode laser sources of various wavelengths. Collimated light from each light source 1736 and 1702a-n is reflected from the surface of a 55/45 beam splitter or beam comber (BC1-BC4).

In some embodiments, the spectrometer may include a white light source. In this configuration, the FC connected fiber from a laser diode source S1 is replaced with a fiber fed light source from a tungsten halogen bulb projecting white light.

Beam combiners 1704a-1704n each may allow some wavelengths to pass while reflecting at least one wavelength (e.g., combining optical wavelengths). In one example, beam combiner 1704a may reflect light at a first wavelength from source 1702a and the beam combiner 1704a may allow other wavelengths to pass through (e.g., light from sources 1702b-1702n). The light from each source may be projected through lens 1706. Lens 1706 may be a collimator to collimate the light received from the light sources.

Reflective surface 1708 and reflective surface 1712 may reflect all light from the sources. In one example, light from sources 1702a-1702n is reflected by reflective surface 1708 through sample chamber 1710. The sample chamber 1710 may contain the sample of a food processing byproduct. In various embodiments, the sample chamber 1710 is or contains a cuvette. In another example, the sample chamber 1710 is or contains transparent substrates to which a sample of a food processing byproduct may be found. The light from the sources pass through the sample chamber 1710 and then is reflected by reflective surface 1712.

The second section of the optical path 1734 propagates the collimated beam through a scattering sample of the sample chamber 1710. In one example, an 8 mm collimated beam from the light source is reflected perpendicularly from reflective surface 1708 through a sample cuvette holder (i.e., the sample chamber 1710). In this example, the entrance aperture of the sample chamber 1710 has a 9 mm diameter. The sample chamber 1710 may contain a sample in a liquid medium and may have a width of 10 mm perpendicular to the beam and a length parallel to the beam of 2 mm.

In one example, the sample chamber 1710 may be filled with approximately 1 ml of liquid so the full 8 mm beam passes through the sample. The residual collimated beam and the light scattered off the sample may then reflect perpendicularly off of reflective surface 1712 and exit to the next section of the optical path.

Lens 1706 may collimate the light and lens 1714 may focus the light on the deformable mirror 1724. Collimated light from the sample chamber 1710 may be incident on lens 1706. Lens 1706 (e.g., f1=75 mm) and lens 1714 (e.g., f2=30 mm) may be separated by a distance D12=f1+f2=105 mm. In this example, the light leaving the lens 1714 is collimated with a beam size of 3.2 mm. The collimated beam is incident on a deformable mirror 1724, which may be a BMC MEMS deformable mirror, composed of, in this example, an equal spaced, 12×12 actuator grid array, where each actuator is separated by 400 microns.

The deformable mirror 1724 may, in some embodiment, may control the wave front of the light based on information received from the wavefront sensor 1730. In this example, the light may magnify and/or enhance the light of the optical path. Control of the deformable mirror 1724 may allow for control of the wave front of the light to direct a flat wave front to the detector 1732. It will be appreciated that, in some examples, the optical path 1700 may not have a deformable mirror 1724. In that case, the optical path 1700 may not have a beam splitter or a wave front sensor 1730.

Light then is further focused by lens 1718 on the vortex mask 1716. The vortex coronagraph may be created by first constructing a 4f beam relay using two matching 75 mm lenses, lens 1718 (f3=75 mm) and lens 1722 (f4=75 mm). Lens 1718 may be placed a distance equal to the focal length of lens 1718 away from the DM (D3=75 mm). Lens 1718 and lens 1722 may be separated by a distance D34=3+f4=150 mm.

In some embodiments, a collection of monochromatic vortex masks (VM) matched to the input laser diodes are loaded into a filter wheel and placed in the focal plane between lens 1718 and lens 1722. The filter wheel may be mounted to a 3-axis translation stage to provide fine position control for vortex mask alignment. In various embodiments (e.g., any of examples depicted in FIGS. 17A-C), the irradiance at the entrance of the vortex mask may be 34 micrometers.

Lens 1722 may be a collimator lens and/or may focus the light on the lyot stop 1720. In this example, a lyot stop 1720 is placed after lens 1722 at a distance of D4=75 mm. Different lyot stop 1720 sizes may be used. In one example, a lyot stop 1720 uses a 0.8×Dpupil˜=2.56 mm aperture.

The lyot stop 1720 may be a lyot mask (e.g., lyot stop) such as a Lyot-plane phase mask, which enables improved contrast performance. The Lyot-plane phase mask may relocate residual light away from a region of the image plane, thereby reducing light noise from the sources of the spectrometer and improves sensitivity to off-axis scattered light.

In between lens 1722 and the lyot stop 1720 a 92/8 beam splitter (BS) is placed in the beam, the 8% reflection is passed into a wavefront sensor 1730, which may be a Shack-Hartmann wavefront sensor, which is also a distance D4=75 mm after lens 1722.

The wavefront sensor 1730 may measure the wave front of the light and control the deformable mirror to flatten the wavefront on the vortex mask 1716 (otherwise signature artifacts may be created).

It may be appreciated that the system may be configured for broadband use by replacing the monochromatic vortex masks with broadband masks that are matched to the new set of narrowband filters in the detector optics.

The residual light that exits the lyot stop 1720 is passed through a circular polarization analyzer 1740 that is matched to the circular polarizer 1738 in the light source system. The light may then pass through a Filter wheel with 10 nm narrowband pass filters 1742 which may have central wavelengths that are matched to the laser diode sources. The residual light may then be focused onto a detector by lens L5 1726 (e.g., f5=7.5 mm). it may be appreciated that the high contrast (>10-4) performance of the light suppression will be limited by the polarization purity of the beam, so care may be taken to maximize polarization purity.

In some embodiments, a linear array may be used if white light is instead used. In this case the detector is replaced with a fiber mounted multi-mode fiber with a fiber core size greater than 10 microns (Typical use is 400 microns). When setup in the white light configuration, the narrowband filters may be set up to have the same bandpass as the broadband.

The detector 1732 detects spectral components (e.g., intensities of received wavelengths). In various embodiments, the detector 1732 is part of a spectrometer, a photodiode, or an LCD camera. The detector may generate measurements indicating intensities of wavelengths from the incoherent light of the optical path. The detector may provide absorption or transmittance measurements related to the particles and components of the sample.

In one example, the detector 1732 is in communication with a processor to assess and generate the measurement results. The measurement results may then be used to identify if the sample contains a pathogen.

The measurement results may be received by a discriminator. A discriminator may categorize or determine if the patron is infected by assessing and/or analyzing the measurement results. The discriminator may assess the measurement results using a logistic regression technique, an AI approach (e.g., convolutional neural network), and/or other statistical methods. In some embodiments, the measurement results may be used to create and/or train the discriminator.

FIG. 17C is another example of an optical path of a spectrometer in some embodiments. In the example described with regarding to FIG. 17C, each component will include a location measured directly to the previous component along the optical path (in the direction against incoming light) and another location measured directly along the optical path to the entrance aperture (e.g., the detector may be 1239.257 mm along the optical path from the entrance aperture 1750). These locations are by way of example. It will be appreciated that the components may be located in many different positions relative to each other, the entrance aperture, and/or the light source.

The path may include an entrance aperture 1750. The entrance aperture 1750 may have a beam aperture. For example, the entrance aperture 1750 may accommodate a beam diameter of 6 mm for a beam of wavelength 635 nm. It may be appreciated that the entrance aperture 1750 may accommodate a beam diameter of any size (e.g., between 4-8 mm) and at any wavelength (e.g., 592 nm-700 nm). The entrance aperture 1750 may be any distance from the light source (e.g., 30 mm).

The polarizer 1752 may be made of any material, such as calcite. The polarizer 1752 may be 63.9463 mm from the light source and 30 mm along the light path to the entrance aperture 1750. The polarizer 1752 may polarize light from the light source received via the entrance aperture 1750.

The quarter wave plate 1754 may reflect light received from the polarizer 1752 to the cuvette 1756. The quarter wave plate 1754 may be 99.978 mm from the polarizer 1752 and 93.9463 from the entrance aperture 1750.

The cuvette 1756 may contain a sample from a patient or user that is to be measured. The cuvette may be located 124.1297 mm from the quarter wave plate 1754 and 193.9243 mm from the entrance aperture 1750.

The quarter wave plate 1758 may receive light received from through the cuvette 1756 and may reflect all or part of the light to lens 1760.

Lens 1760 may receive light from the quarter wave plate 1758 and allow the light to pass to the lens 1762. The lens 1760 may include, for example, a first side surface radius of curvature 108.07 mm and the other surface (the second side) may be plano. In this example, the lens 1760 may have a thickness of 10 mm and be made of a material such as N-Bk7. It will be appreciated that the surface radius of curvature may be many different sizes (e.g., 90 to 120 mm), the other surface may be plano or curved, the lens 1760 may have any different thickness (e.g., 8-12 mm), and be made of any material or combination of materials. The lens 1760 may be 318.28 mm from the cuvette 1756 or the quarter wave plate 1758. The lens 1760 may be 318.054 from the entrance aperture 1750.

Lens 1762 may receive light from the lens 1760 and allow the light to pass to the deformable mirror 1764. The lens 1762 may include, for example, a first side being plano and a second side having a surface radius of curvature −57.64 mm. In this example, the lens 1762 may have a thickness of 10 mm and be made of a material such as N-Bk7. It will be appreciated that the surface radius of curvature may be many different sizes (e.g., −45 to −75 mm), the other surface may be plano or curved, the lens 1762 may have any different thickness (e.g., 8-12 mm), and be made of any material or combination of materials. The lens 1762 may be 93.9994 mm from the lens 1760. The lens 1762 may be 636.582 mm from the entrance aperture 1750.

Deformable mirror 1764 may receive light from the lens 1762 and project the light to the lens 1766. The deformable mirror 1764 may be 78.834 mm from the lens 1762 and may be 760.5814 mm from the entrance aperture 1750.

Lens 1766 may receive light from the deformable mirror 1764 and allow the light to pass to the vortex mask 1768. The lens 1766 may include, for example, a first side having a surface radius of curvature 38.6 mm and a second side being plano. In this example, the lens 1766 may have a thickness of 10 mm and be made of a material such as N-Bk7. It will be appreciated that the surface radius of curvature may be many different sizes (e.g., 25 to 55 mm), the other surface may be plano or curved, the lens 1766 may have any different thickness (e.g., 8-12 mm), and be made of any material or combination of materials. The lens 1766 may be 76.3095 mm from deformable mirror 1764. The lens 1766 may be 805.4154 mm from the entrance aperture 1750.

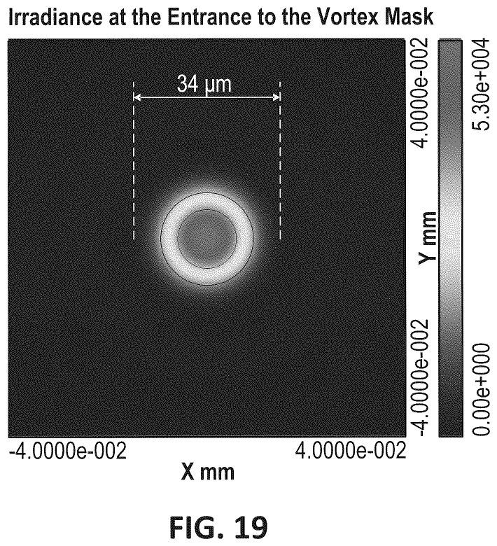

The vortex mask 1768 may receive light from the lens 1766 and allow (at least some) of the light to pass to lens 1770. The vortex mask 1768 may be 72.0435 mm from the lens 1766 and may be 881.7249 mm from the entrance aperture 1750. FIG. 19 depicts the irradiance at the entrance to the vortex mask 1768 is 34 micrometers in one example.

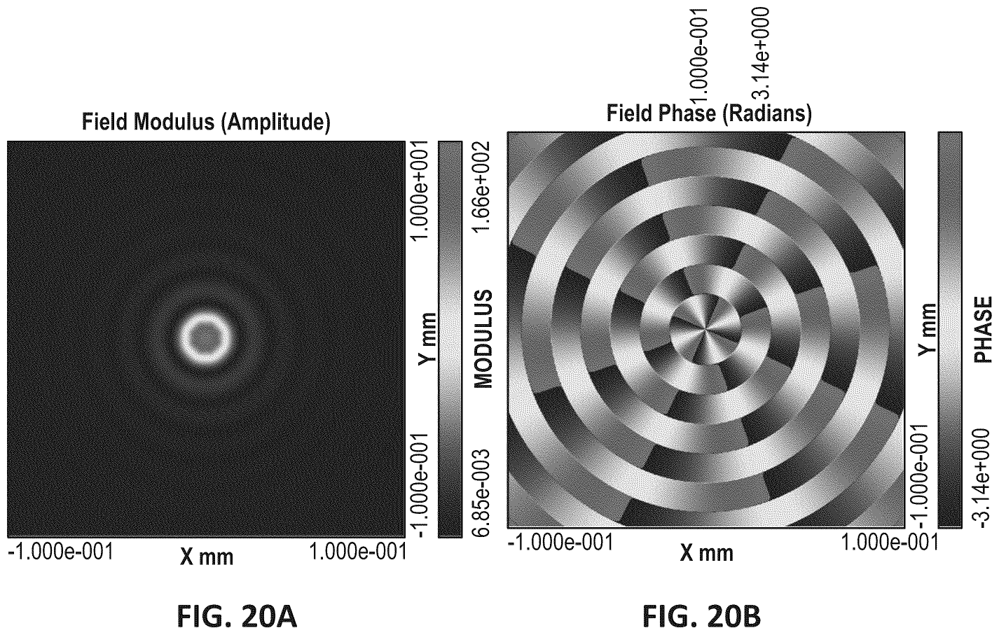

FIGS. 20A and 20B depicts modulus and phase of the field after the vortex mask 1768 in some embodiments. FIG. 20A depicts a field modulus (amplitude) after the vortex mask 1768 in some embodiments. FIG. 20B depicts a field phase (radians) after the vortex mask 1768 in some embodiments.

Lens 1770 may receive light from the vortex mask 1768 and allow the light to pass to the lyot stop 1772. The lens 1770 may include, for example, a first side being plano and a second side having a surface radius of curvature −38.6 mm. In this example, the lens 1770 may have a thickness of 10 mm and be made of a material such as N-Bk7. It will be appreciated that the surface radius of curvature may be many different sizes (e.g., −30 to −45 mm), the other surface may be plano or curved, the lens 1770 may have any different thickness (e.g., 8-12 mm), and be made of any material or combination of materials. The lens 1770 may be 78.934 mm from the vortex mask 1768. The lens 1770 may be 953.7684 mm from the entrance aperture 1750.

The lyot stop 1772 may receive light from the lens 1770 and allow (at least some) of the light to pass to beam splitter 1774. The lyot stop 1772 may be 57.1156 mm from the lens 1770 and may be 1,032.702 mm from the entrance aperture 1750.

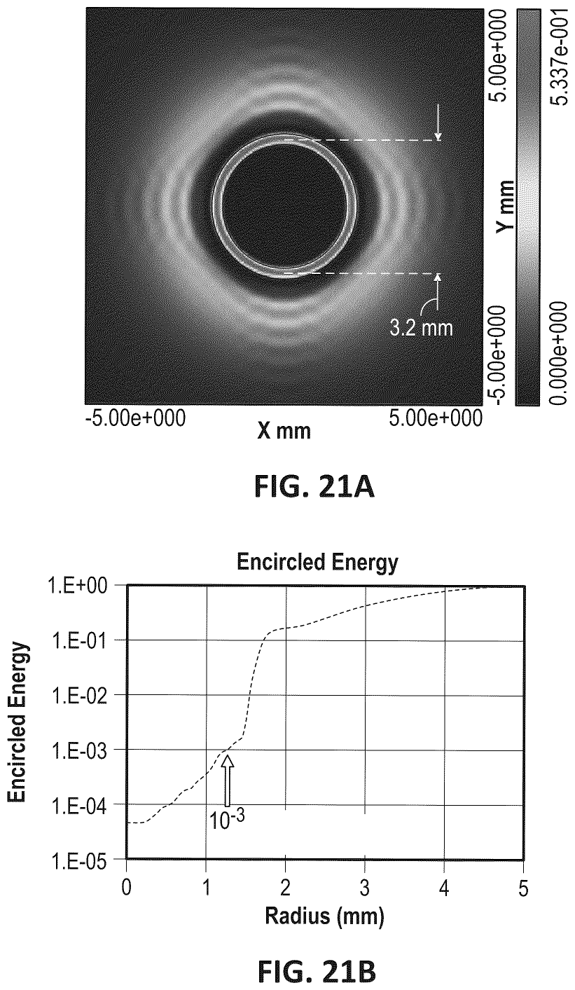

FIGS. 20A and 20B depicts interior irradiance at the lyot stop 1772 in some embodiments. The vortex mask 1768 may produce a “ring of fire” at the lyot stop plane. The interior irradiance may be approximately 10-4 of the ring irradiance and the total power may be, for example, 9.33. FIG. 21A depicts an example interior irradiance of the lyot stop 1772 in one example. FIG. 21B is a graph indicating a 10-3 contrast for a lyot stop radius of 1.25 mm in one example.

The beam splitter 1774 may receive light from lyot stop 1772 and allow (at least some) of the light to pass to polarizer 1776. The beam splitter 1774 may be 68.7634 mm from the lyot stop 1772 and may be 1,089.818 mm from the entrance aperture 1750. The beam splitter 1774 may be configured to measure all or some of the received light, compare the characteristics to criteria or a reference, and control the deformable mirror 1764 to control the light beam.

The polarizer 1776 may receive light from beam splitter 1774 and allow the light to pass to lens 1778. The polarizer 1776 may be 50 mm from the beam splitter 1774 and may be 1,180.581 mm from the entrance aperture 1750.

Lens 1778 may receive light from the polarizer 1776 and allow the light to pass to the detector 1780. The lens 1778 may include, for example, a first side having a surface radius of curvature 8.89 mm and a conic constant of −0.717. The second side may be plano. In this example, the lens 1778 may have a thickness of 2.5 mm and be made of a material such as N-SF11. It will be appreciated that the surface radius of curvature may be many different sizes (e.g., 2-15 mm), the other surface may be plano or curved, the lens 1778 may have any different thickness (e.g., 1-5 mm), and be made of any material or combination of materials. The lens 1778 may be 8.676 mm from the polarizer 1776. The lens 1778 may be 1,230.581 mm from the entrance aperture 1750.

The detector 1780 may receive light from the lens 1778. The detector may be or include a camera such as a CCD. In this example, the detector 1780 may be 1,239.257 mm from the entrance aperture 1750.

In various embodiments, the vortex spectrometer or digital device may perform dark noise correction to reduce noise. Dark noise arises from changes in thermal energy of the spectrometer and/or camera (e.g., detector). The increase of signal also carries a statistical fluctuation known as dark current noise.

As discussed herein, dark noise arises from variation a cross an imaging sensor with no external illumination. Dark noise may be corrected by taking a reference with no illumination, calculating the mean of the dark noise signal, and then subtracting the dark noise off of the sample signal.

FIG. 22 is a flowchart 2200 for identifying pathogens from spectrometer data in some embodiments. In some embodiments, a spectrometer as discussed herein may take measurements of a food processing byproduct. The measurements may then be analyzed to detect pathogens. Different pathogens may produce different wavelength intensities. As a result, a pathogens may be associated with a “signature” or “thumbprint” of spectral intensities that may be detected.

In step 2202, a digital device may receive spectrogram data from a spectrometer as discussed herein (e.g., with or without a vortex spectrometer and lyot stop, including, for example, the spectrometer depicted in FIG. 17A, 17B, or 17C). The digital device may be local or remote to the spectrometer that produced the spectrometer results. In one example, the spectrometer may be a health screening system as discussed herein. The digital device may receive raw spectrogram data or spectrogram data after transmission and reconstruction.

In step 2204, the digital device may perform dark noise correction. Optical experiments are made variable by imperfections in the light source, transmission of the optical path, and possibly wavelength-specific non-idealities in the detector. For each sample measured, a spectrometer may be configured to collect multiple references (e.g., two) references which control for variance in the environment in which the spectrometer is placed, and in the measurement hardware. Data collected in a reference frame, TR, together with the dark frame TD and the collected sample TS, allows the formation of the attenuance:

μ=(T_{S}−T_{D})/(T_{R}−T_{D})

In one example, raw transmission spectra may be collected during a single 100 μs acquisition for the purpose of calibrating measurement response. In this example, two references are collected: 1) a dark frame which is acquired with the light-source for the detector turned off and 2) a reference collected with a cuvette for the sample in place and filled with DI water, the diluent used in the assay. The dark frame allows for the subtraction of environmental noise due to imperfect isolation of the interaction medium and sample during measurement. The reference may be utilized in order to measure the attenuation due to the presence of the cuvette and diluent medium, and to form the absorbance according to −TS/TR.

The attenuance is a physical property of the analyte which does not depend on the variable aspects of the measurement. To ensure good correspondence between the measured sample attenuance and the physical value, the assay may specify a collection of the reference frame immediately before collection of the data from the clinical sample or analyte.

Measurements of dark noise may be made using digital numbers. Digital numbers are assigned to a pixel in the form of a binary integer, often in the range of 0-255 (a byte). A single pixel may have several digital number variables corresponding to different bands recorded.

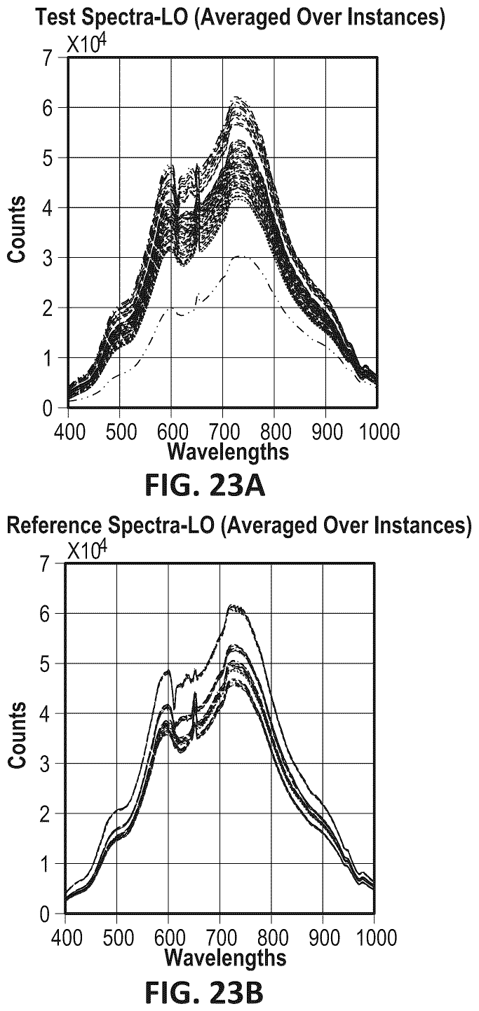

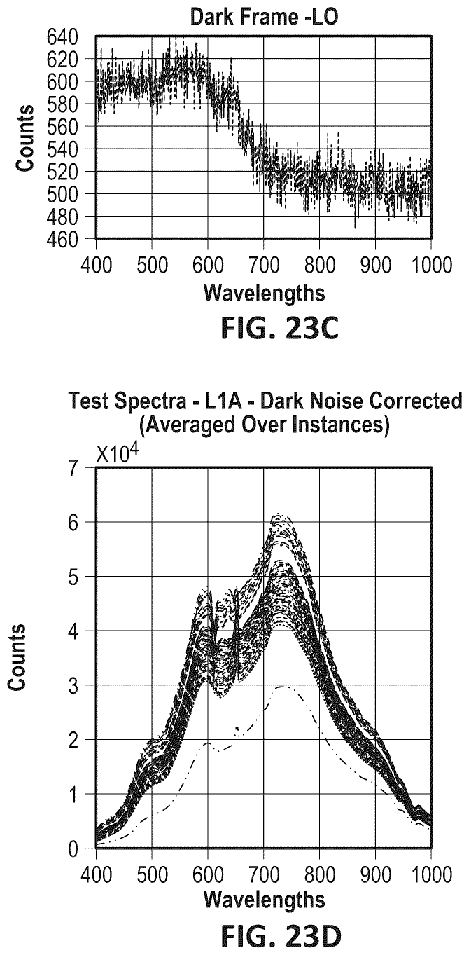

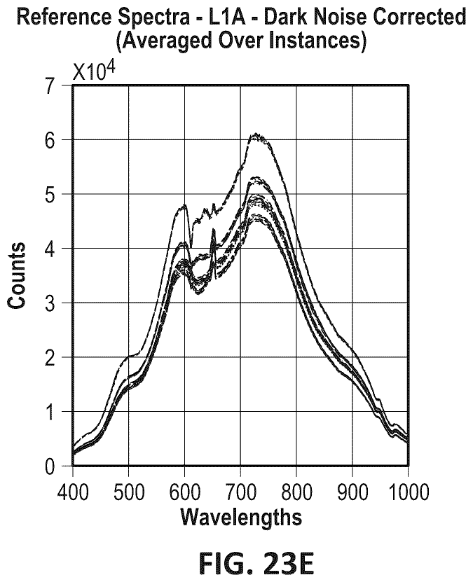

FIG. 23A depicts a test spectra and FIG. 23B depicts a reference spectra in two examples. Here, the shape of the spectra is observed, and the signal may be, in this example, about 60,000 digital numbers. The resulting dark noise in comparing the reference to the test has a mean value of about 600 digital numbers. FIG. 23C depicts the mean value of the dark noise in one example.

It will be appreciated that the dark noise for a particular spectrometer may not change. As a result, the spectrometer may be tested in a factory to identify dark noise and then a dark noise correction may be applied to spectrogram data throughout the day or going forward. In some embodiments, the spectrometer may be tested daily or at some other periods of time, and then the dark noise detected during testing may be used to correct spectrogram data.

In various embodiments, the dark noise caused by the spectrometer may be filtered from the data. By identifying dark noise and filtering the dark noise from the spectrometer data, the signal (e.g., meaningful spectral intensities) may be boosted.

In various embodiments, the dark noise of a particular spectrometer may be measured. This may be done by letting the spectrometer warm up and measuring water and/or a common transport medium. Noise caused by thermal changes may be detected by the detector (e.g., by a CCD camera). Multiple measurements may be taken (e.g., at the same time or over time) and the dark noise may be averaged, aggregated, and/or otherwise collected.

FIG. 23D depicts a test spectra of dark noise corrected in one example. FIG. 23E depicts a reference spectra of dark noise corrected in one example.

In step 2206, the digital device performs spectrogram normalization. Variations from sample to sample may create issues. In some embodiments, an autoexposure is used. For example, the digital device and/or the spectrometer may take an image of the spectral intensities and determine location in a fixed integration of time and determine the integration time to get to a desired measurement (e.g., 60,000 digital numbers).

In some embodiments, reference data may be taken (e.g., by using the spectrometer on water or VTM) and a location of a peak intensity identified. The digital device may scale the spectral intensities from that wavelength. The reference information may be taken using water or a VTM to determine peak intensity. The reference may be taken at the factory, once a day, or at any time.

This correction may assist flat fielding of the CCD camera where some pixels are not as sensitive as other pixels in the CCD camera (which as a result, may detect information that is not caused by differences in intensity but rather differences in chip sensitivities).

For example, a determination of where a peak occurs in the reference may be performed. Then all references may be scaled to that peak intensity.

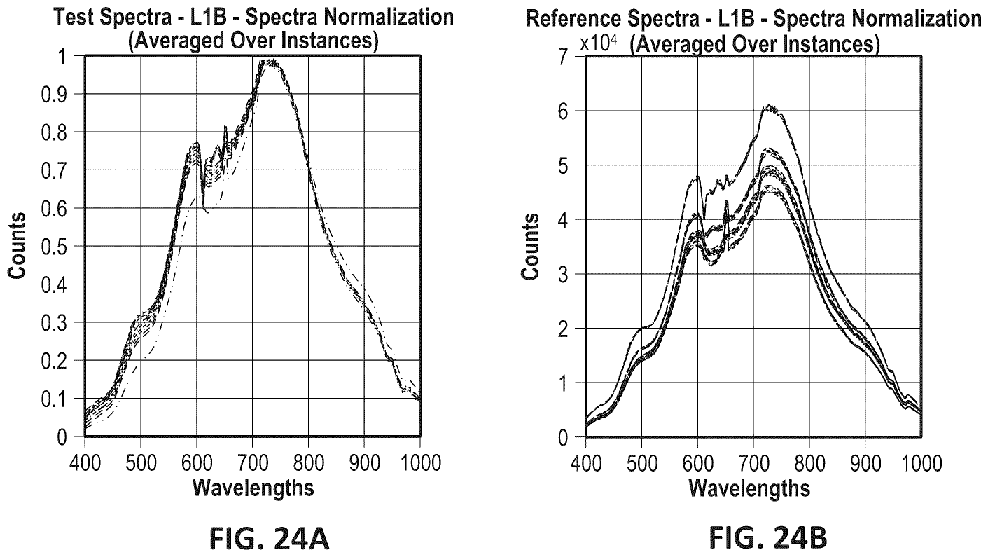

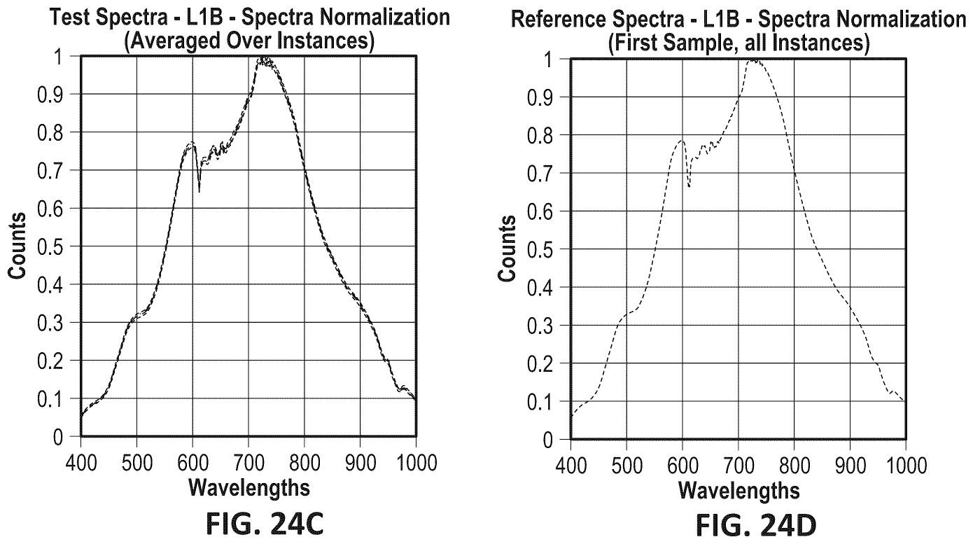

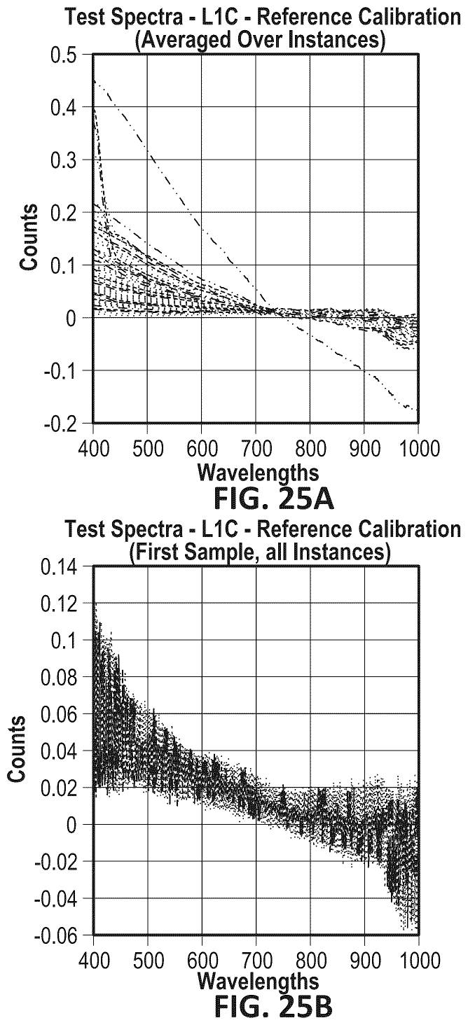

FIG. 24A depicts an example test spectra including spectra normalization averaged over instances. FIG. 24B depicts an example reference spectra including spectra normalization averaged over instances.

FIG. 24C depicts a test spectra with spectra normalization for the first sample, all instances. FIG. 24D depicts an example reference spectra including spectra normalization for the first sample, all instances.

In step 2208, the digital device performs reference calibration. In one example, the digital device takes the ratio of the reference to the signal and then subtracts the reference. The curve may be characteristic of the substance. A flat line would indicate no information.

FIG. 25A depicts an example test spectra including spectra normalization averaged over instances. FIG. 25B depicts an example reference spectra including spectra normalization averaged over instances.

In step 2210, the digital device performs background removal and estimation. In one example, the digital device takes the ratio of the reference to the signal and then subtracts the reference.

It will be appreciated that samples are often more negative (uninfected) than positive. For example, the positive rate may be only 5% or less of all samples (e.g., 20 times more negatives than positives). In various embodiments, a background pool is created. Negative results may be clustered into families.

In various embodiments, the digital device groups results according to similarities. For example, the digital device may select two negative results and subtract them to get a minimum energy which may be used for a characteristic curve. In some embodiments, measurements of any number of samples may be divided into levels (e.g., based on similarities and/or measurements). There may be any number of levels. For example, similarities or measurements may be ordered or ranked based on intensity, energy, and/or wavelength. The ordered or ranked information may be divided into sets based on equal or unequal thresholds.

Each of the measurements or sets may be compared to each other and a minimum may be taken to get characteristics for each level. A pool of negatives (compare positive to negative) may be obtained. A pool of negatives refers to a collection of negative results (e.g., no infection indicated) as opposed to positive results (e.g., infection indicated).

The result may be assessed to determine the curve. A flat line, for example, may contain no information while a curve may indicate information related to virus infection. The digital device may remove the background from future signals/measurements to remove the background signature of saliva and VTM itself. The background pool of information may also be determined and minimized to find the minimum energy.

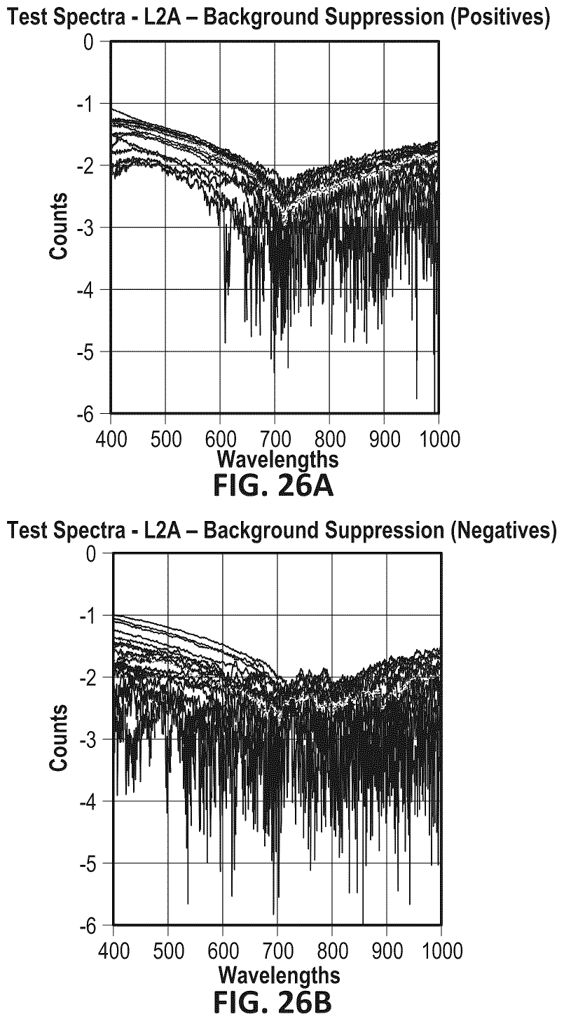

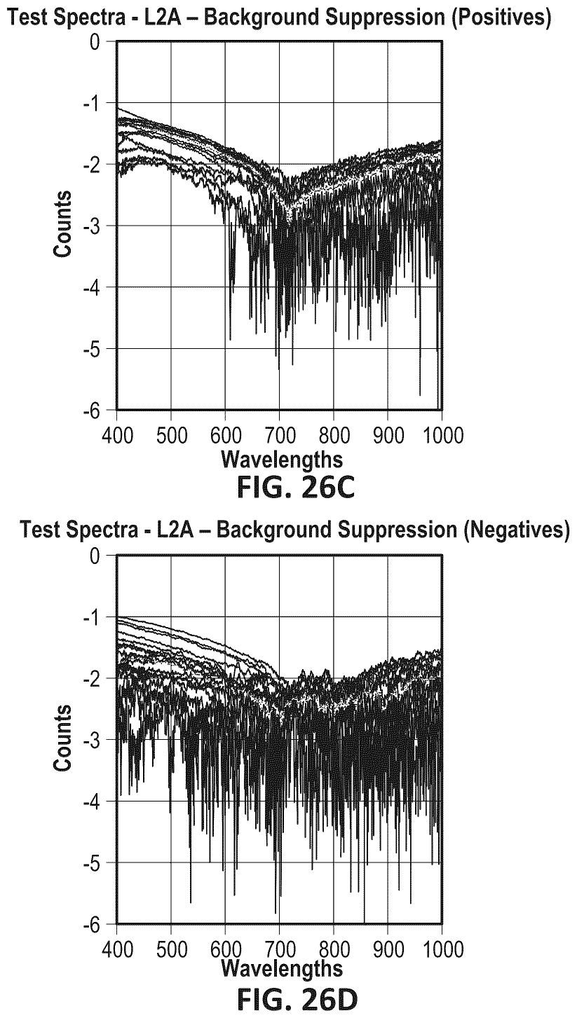

FIG. 26A depicts an example test spectra of positive (infection) results with background suppression. FIG. 26B depicts an example test spectra of negative (infection) results with background suppression.

FIG. 26C depicts an example test spectra of positive (infection) results with background suppression. FIG. 26D depicts an example test spectra of negative (infection) results with background suppression.

In step 2212, the digital device may perform lucky imaging background minimization. In various embodiments, the digital device and/or spectrometer may make many measurements of a sample. The digital device may assess the different samples to identify the sample that provides the most energy. For example, the digital device may perform background estimation and removal from any number of images (e.g., all or a subset) to identify the results that express the most information or an indication of a positive or negative result.

In step 2214, the digital device may perform wavelet scalogram conversion. In various embodiments, the digital device performs a wavelet decomposition. A wavelet may be selected, and a cross correlation performed along the signal to measure intensities (e.g., weight on left of graph and wavelength along the X axis).

With background estimation, the difference between negatives and positives can be depicted. Intensity variations appear in high frequency wavelets which may indicate a spectral signature for infection (e.g., covid-19) in step 2216.

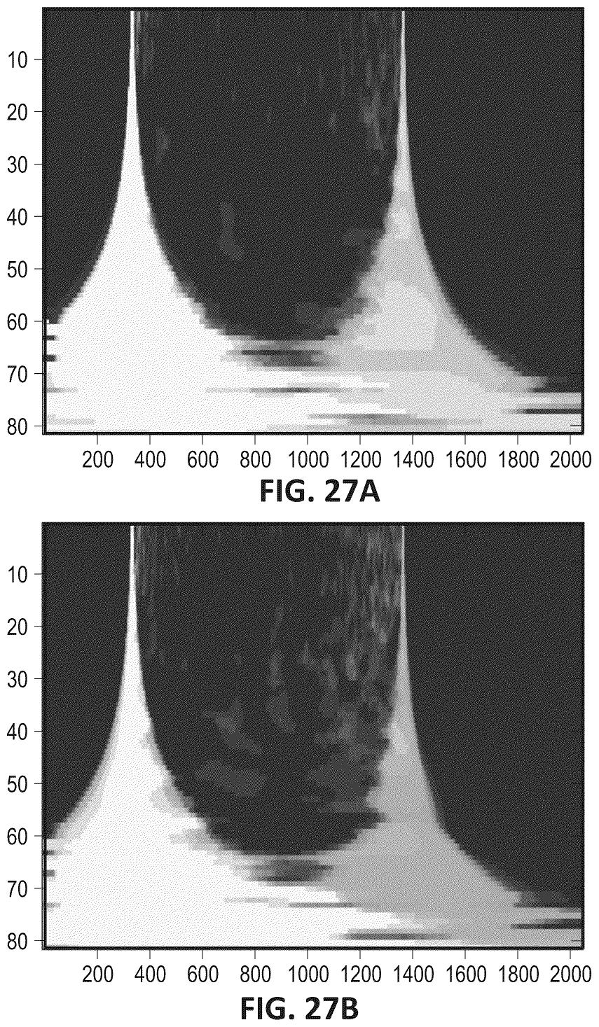

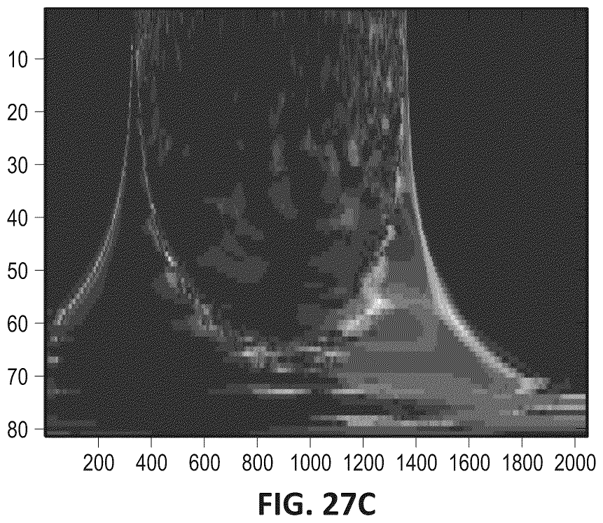

FIG. 27A depicts a negative result scalogram conversion after wavelet correlation. FIG. 27B depicts a positive result scalogram conversion after wavelet correlation. FIG. 27C depicts a difference between the positive and negative result scalogram conversion depicting the difference and indicating the signature of infection.

In various embodiments, the digital device may perform scalogram conversion after background removal to identify if the signature (e.g., intensities of absorption lines associated with a particular infection, virus-related protein, or virus) or pattern is present. In various embodiments, the digital device may perform the inverse wavelength transform.

Variations from sample to sample may create issues. In some embodiments, an autoexposure is used. For example, the digital device and/or the spectrometer may take an image of the spectral intensities and determine location in a fixed integration of time and determine the integration time to get to a desired measurement (e.g., 60,000 digital numbers).

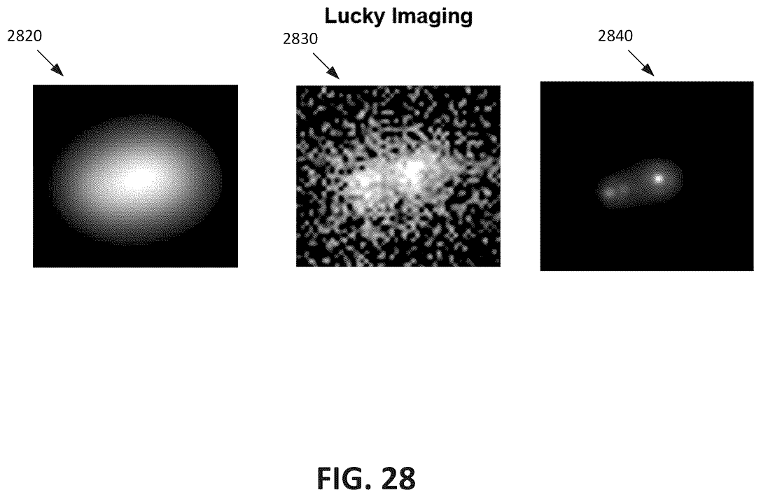

FIG. 28 depicts examples of lucky imaging in some embodiments. In various embodiments, the spectrometer with a vortex mask and/or a lyot stop may take multiple measurements of the same sample. The spectrometer or processor may select one or more images containing information most indicative of the presence of the virus (e.g., the spectral signature of the virus) or lack of presence of the virus. For example, luck imaging may utilize multiple measurements to select the image with the best relative clarity and accuracy (e.g., images that depict the energy for the wavelengths of interest associated with a virus). FIG. 28 depicts spectrometer output image 2820 which is improved using lucky imaging to rendered image 2830 which is further improved through lucky imaging to image 2840. There may be any number of measurements used for lucky imaging.

Combined with lucky imaging, a signal may be strengthened by processing many spectrogram snapshots together. In one example, multiple snapshots may be taken of the sample using a spectrometer with a vortex mask 1540 as discussed herein. Lucky imaging enables using multiple measurements to improve clarity, reduce noise, and detect previously faded signals related to pathogens, especially pathogens that are relatively small, such as viruses.

A discriminator may also be used, in some embodiments. The discriminator may receive results from the spectrometer, assess the information, and provide an indication based on the results (e.g., classification of infection or not infected). In one example described herein, scalograms are collected and parts of the scalograms (e.g. the parts associated with the signature of the virus being tested for) may be compared against references or thresholds. Based on the comparison, the discriminator (e.g., classifier) may provide an indication of infection or not infected (or indeterminant).

In other embodiments, a convolutional neural network (CNN) may be used as a discriminator to identify measurements indicating infection and non-infection. In various embodiments, a neural network may be trained using measurements from the vortex spectrometer as discussed herein. The neural network may also be trained using laboratory test results to confirm those patrons that are infected and those that are not infected. The neural network may receive or generate a set of features based on the output (i.e., measurement results) of the vortex spectrometer. The neural network may then be tested to confirm predictions against known infection/noninfection results.

In one example, the neural network may identify wavelength intensities in the ranges of 735 nm 780 nm 1510 nm, and 1560 nm as being indicative of infection.

It will be appreciated that any discrimination may be utilized to identify infection and noninfected patrons and/or samples. For example, any statistical method, such as logistic regression analysis, may be utilized.

In various embodiments, an algorithm may be utilized to identify spectral wavelengths and/or scalograms associated with specific pathogens or chemicals to be identified. For example, specific approaches may be utilized in conjunction with many samples to create a “fingerprint” of a scalogram or set of spectral wavelengths associated with a specific pathogen. Scalograms and/or spectral wavelengths generated from a patient's sample may be compared to the fingerprint, reference scalogram, and/or reference set of spectral wavelengths to determine infection or likelihood of infection.

It will be appreciated that many different algorithms and/or approaches may be used. For example, a logistic regression may be used, a K-nearest neighbors approach, decision trees, or random forest. In some embodiments, each sample may be divided by the medium of all collection instances, the data is normalized (e.g., mean=0, std=1), and classification is performed using a logistic regression.

In some embodiments, different approaches may be used with different spectrometers and/or depending on what is being tested. For example, a different dataset from a different spectrometer may utilize an approach where each sample may be divided by the medium of all collection instances, the data is normalized (e.g., mean=0, std=1), and classification is performed using a random forest. Another example may utilize a different approach where, for each sample, an average over windows of a predetermined (e.g., 20) consecutive instances is taken, normalization is performed to sample absorption (e.g., (sample-reference)/reference), and classification is performed using a random forest.

As discussed herein, the vortex spectrometer may be utilized to detect pathogens and/or spectral signatures of interest (e.g., to detect substances in food, chemicals, or the like). As discussed herein, when light is passed through a liquid, photons at specific wavelengths may be absorbed by particles in the liquid. The exact wavelengths where absorption occurs depend on the exact chemical composition of the absorbing particles. In this way, a spectrometer can measure the presence of a specific particle if its spectral absorption characteristics are known. The amount of absorption that occurs depends on the number of absorbers that are within the path of the light passing through the test chamber. Therefore, the absorption may also be dynamic if the particles transit the light path due to their motion within the liquid. Particles may be in motion for a variety of reasons including diffusion, convection, settling, and/or the like.

Particles of a substance in motion change the absorption profile dynamically as particles move in and out of a beam path. Consider the case of a spectral characterization of a dynamic absorber in an inert liquid, where the liquid does not contribute to the spectral signature:

I_{S}(λ,t)=I₀(λ,t)−N_{A}(t)·I_{A}(λ)

Where the light source is assumed to have a dynamic spectral response given by I₀(λ, t), N_{A}(t) is the number of dynamic absorbers of substance A, and substance A has an absorption spectrum given by I_{A}(A). The light source may vary dynamically for a variety of reasons including shot noise, dark noise, thermal drift, and the like. In many cases this variation may be greater than the absorption signature from substance A. However, these noise variations are derived from different physical processes and have different stochastic characteristics, which we may exploit to differentiate between light source variations and dynamic absorber concentration variations.

Light source variations are random and uncorrelated in time but variations in particle density are not. Particle variations are partially correlated in time and depend on several factors such as: particle size, liquid viscosity, temperature etc.

Consider the autocorrelation of a time series:

$$autocorr ( X , τ ) = \frac{{\sum }_{i = 1}^{n} [ {X}_{i} - \overline{X} ] [ {X}_{i - τ} - \overline{X} ]}{{{\sum }_{i = 1}^{n} [ {X}_{i} - \overline{X} ]}^{2}}$$

Where the time series is correlated with itself delayed in time by a time lag, τ, there are n discrete time steps and X̅ is the mean of the time series. Applying equation 2 to equation 1 and using the fact that the autocorrelation of the sum of two uncorrelated functions is the sum of the autocorrelation of the functions separately yields:

autocorr(I_{S}(λ,t),τ)=autocorr(I₀(λ,t),τ)−autocorr(N_{A}(t),τ)·I_{A}(λ)

If the light source fluctuation is dominated by a Poisson process such as shot noise, then the autocorrelation may take the form:

autocorr(I₀(λ,t),τ)=|I₀(A)|_{Δt}·Δt+|I₀(λ)|_{Δt}2·Δt·(Δt+τ)

If we normalize the autocorrelation to the unshifted case, τ=0, the autocorrelation may no longer be wavelength dependent. In other words, the entire spectrum undergoing a poisson random process will have the same identical normalized autocorrelation function. Variability in the autocorrelation function with wavelength can only be due to dynamic absorbers. In addition, all dynamic absorbers composed of the same substance will produce the same autocorrelation function, which will differ from the autocorrelation function of a poisson process, unless the fluctuation in the number of absorbers is also a poisson process, which is not typically the case.

A dip in the intensity of a measured spectra indicates the presence of an absorber that is absorbing light at the wavelength centered on the location of the dip in the spectrum. The dip not only affects the amount of light delivered, the dip has structure itself. The spectral structure of an absorption line may be unique to the underlying absorption process that's creating the measured absorption. A continuous wavelet transform (CWT) is one way to represent spectral structure that naturally filters spectral slowly varying features from those with spectrally faster varying signals. Therefore, the spectral absorbance may be represented as a continuous wavelet transform. Performing a continuous wavelet transform on a dip location in a spectrum will provide spectral structure related to the spectral distribution of the light around a given dip location. Some dips may be described as sharp, others as shallow. These differences in dip shape may also provide a set of characteristic features that could uniquely identify a specific internal state that produces a given measured response.

By measuring the location and shape of absorption dips, a specific unique state may be specified. Using the wavelet decomposition as a measure of the shape of a spectral peak, a state may be quantified based on the spectral bins and their structural shape described by N_{V} complex voices.

\vec{x}(λ,2·N_{V}+1)

First, the full state space may be divided into two states: a state \vec{x}_{A}, when substance A is present and state \vec{x}₀, when it is absent.

\vec{x}(λ,2·N_{V}+1)=\vec{x}_{A}(λ,2·N_{V}+1)+\vec{x}₀(λ,2·N_{V}+1)

In this example, there is an assumption that the spectral measurements are collected from a sample pool where the only difference between \vec{x}_{A} and \vec{x}₀ is the presence of substance A. Given a set of known measurements, of states \vec{x}_{A} and \vec{x}₀, a Kalman filter may be used to estimate the states \hat{x}_{A} and \hat{x}₀, based on a series of measurements:

$${z}_{n} ( λ , features ) = {\sum }_{i = 1}^{i = {λ}_{\dot{band} / Δλ}} {z}_{n} ( {λ}_{i} ; \vert I \vert , Re \{ {f}_{1} \} , Im \{ {f}_{1} \} , ... Re \{ {f}_{{N}_{V}} \} , Im \{ {f}_{{N}_{V}} \} )$$

The Kalman filter algorithm may be split into two stages: Prediction and Update. During the prediction stage, a prediction of the state of the system is made based upon a current understanding of the state model.

Predicted state estimate:

\hat{x}ₖ⁻=F\hat{x}ₖ₋₁⁺+Buₖ₋₁

Predicted error covariance:

Pₖ⁻=FPₖ₋₁⁺F^{T}+Q
During the update stage, a measurement may be made, and the state estimate is updated. The update may have a measurement residual given by:

\tilde{y}ₖ=zₖ−H\hat{x}ₖ⁻

And a Kalman gain

$${K}_{k} = \frac{{P}_{k}^{-} {H}^{T}}{R + {HP}_{k}^{-} {H}^{T}}$$

After a measurement, the state estimate may be updated:

\hat{x}ₖ⁺=\hat{x}ₖ⁻+Kₖ\tilde{y}

And the state estimate's error covariance may also be updated:

Pₖ⁺(I−KₖH)Pₖ⁻

A Kalman filter estimates the state of a system given a set of known measurements. If a Kalman filter were applied to two different training sets, it would determine two different estimated states. Given the above equations, the goal is to minimize the estimated error covariance of updating each state with this new measurement. The distribution with the least negatively impacted covariance matrix is the best fit. This may also include cases where one selection improves the state estimate. Either way, with more measurements the Kalman filter may continue to improve discrimination.

Once a model is properly trained with positive and negative states, inferences may begin directly. The point of the inference measurement is to determine if a sample contains substance A. We can make those inferences (with large uncertainties) from the very first measurement. The error variance will improve if the system is properly specified initially. We can determine this based upon the expected update to each state's covariance given the measurement as an update. As we better determine the state of the system through many measurements, we get a better understanding of substance A and how to find it in a large selection of data.

Projecting light at one or more different wavelengths through a disordered medium may provide accuracy improvements in measurements of the wavelength(s). For example, light projected through a diversifier (e.g., speckle pattern or diffuser) causes the light to scatter as light impacts occlusions or marks in the diversifier (e.g., in the speckle or diffuser). The scattering is dependent (at least in part) on the wavelength of the light. The scattering caused by the speckle/diffuser is linear and reproducible. As such, the scattered light caused by the diversifier may be measured and patterns of spectral intensities may be associated with one or more different wavelengths projected by a light source. Based on these associations, spectral intensities may be reconstructed.

By reconstructing the spectral intensities, noise present during a pattern recognition phase may be omitted from the reconstruction. It will be appreciated that noise may be caused by temperature, defects in the light path, the light source, and/or other sources between the light source and detector. Unlike wavelengths from the light source that, when scattered by the diversifier, generate reliable, repeating patterns, noise is inconsistent and may be omitted during spectral reconstruction.

Filtering information may be generated based on spectral reconstruction. The filtering information may be used to remove noise (e.g., noise that is inconsistent with the expected patterns caused by the diversifier).

The system may subsequently be used to test for pathogens in samples from patients. Light at known wavelengths may be projected through a cuvette containing a biological sample. The system may include all or some of the same light source, light path, diversifier, detector, and the like used for spectral reconstruction. By using the filtering information and/or spectral reconstruction to reduce or ignore noise, signals caused by scattering of pathogens in the signal may be detectable. It will be appreciated that the size of certain pathogens (e.g., covid-19) may create spectral signals that would otherwise be subsumed by noise and/or other spectral intensities if not for spectral reconstruction.

In various embodiments, using the filtering information and systems described regarding spectral reconstruction, numerous samples containing known pathogens (e.g., covid-19) may be tested to identify spectral signatures associated with the specific pathogen. Various embodiments may use artificial intelligence as described herein to identify the spectral signature of the pathogen in view of known wavelengths of the light source applied during testing.

The spectral signature, as discussed herein may become a fingerprint for one or more pathogens. After a pathogen spectral signature is obtained, samples may be tested to determine whether the pathogen is present (e.g., the user that provided the sample is infected with the pathogen). In various embodiments, once filtering information is generated for a system, the system may then be used to test one or more samples from the user. It may be unknown if the one or more samples contain any of the pathogen being tested. Using the filtering information, the spectral intensities of the result (after passing through the sample) may be determined and then compared to the pathogen spectral signature (e.g., filtering out noise and/or the spectral intensities of the light source). If the pathogen spectral signature is detected, the system may indicate a positive finding of the pathogen and/or information regarding the spectral intensities detected.

Many of the examples discussed herein contemplate one pathogen being detected. It will be appreciated that any number of pathogens may be detected (e.g., by comparing results from a sample against any number of different pathogen spectral signatures).

It will also be appreciated that detection may occur using a cloud-based system. In various embodiments, a spectrometer with a diversifier may be tested to determine the impact of the diversifier on a light path with a controlled light source. Filtering information may be obtained based on the testing (as discussed herein) and applied using the spectrometer, a digital device local to the spectrometer, or in the cloud. Subsequently, future testing of different patient samples using the same spectrometer may be assessed using the filtering information and pathogen spectral signature(s) using a processor associated with the spectrometer, a digital device local to the spectrometer, or in the cloud.

By using a cloud system to compare results from any number of spectrometers, pathogen spectral signature(s) may be updated, corrected, improved, and/or modified in a central system (as opposed to attempting to provide pathogen spectral signatures to thousands or hundreds of thousands of devices proximate to spectrometers around the world). Further, the system may continue to learn from measurements and samples to improve detection, signature generation, and the like. Further, new variations may be more quickly assessed.

Light may be transmitted through the diffuser or speckle pattern to a detector. Temperature of the spectrometer may be controlled during testing. Similarly, the current and wavelength of the light source may be controlled during detection of the scattering of light (e.g., caused by the diffuser or speckle). This may allow capturing of measurements of the diffusion or speckle as a function of wavelength and current. These measurements may be taken with or without the light passing through a cuvette containing a medium (e.g., water or VTM but not a biological sample).

Patterns may be recognized in many different ways. In various embodiments, artificial intelligence may be used, such as a convolutional neural network or a deep neural network, to identify patterns of scattering associated with different current and/or wavelengths.

Once measurements are taken, filtering information may be generated. The filtering information is information that allows for the spectrometer and/or digital device to remove noise in the system (e.g., within the spectrometer) to better detect signals (e.g., spectral intensities) caused by pathogens caught in the light path, thereby increasing the signal-to-noise ratio and/or improving the accuracy of the spectral measurements. In one example, light from a light source may pass through the sample before being projected through the diffuser or speckle pattern. The detector may then take spectral data measurements of the light and utilize weighting, filtering, or the like (e.g., using the filtering or measurements determined without the sample) per wavelength to reduce noise in the measurements.

In one example, a deep neural network may assist in spectral reconstruction. In some embodiments, a deep neural network (DNN) may be trained to identify impact of the speckle or diffuser on light from a controlled light source by simultaneously measuring a variable control input and a signal output. In one example, this may be accomplished by simultaneously measuring a speckle field with a 2D pixel array and measuring a spectrum while modulating the spectrum of the light source. The DNN may be trained to identify wavelengths of the light source by simultaneously (or near simultaneously) measuring the current applied to a light source (e.g., a 635 nm red LED light source) and the output of a spectrometer (e.g., a VIS-NIR spectrometer).

DNN-assisted spectral reconstruction may be performed with a wide variety of variable input controls.

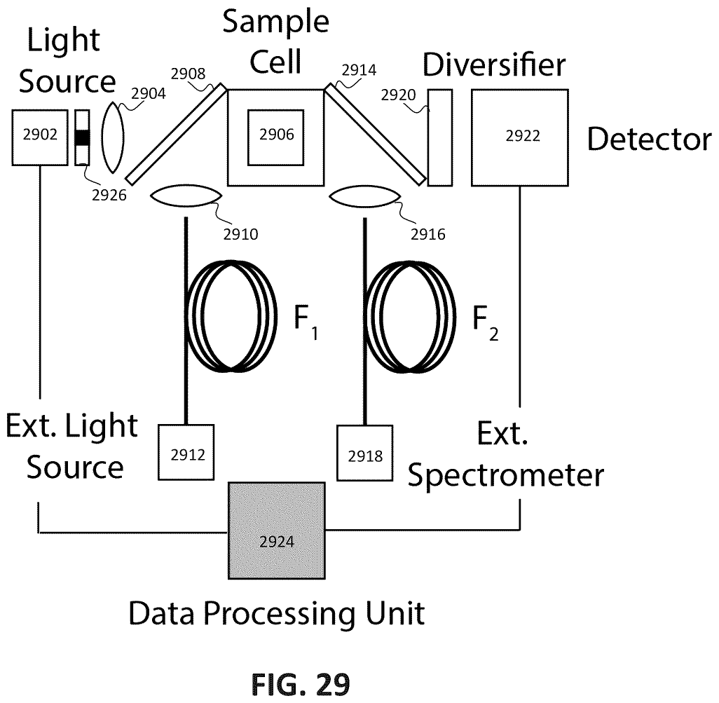

FIG. 29 depicts a system for spectral reconstruction based on scattered light caused by a diversifier in some embodiments. The system depicted in FIG. 29 may be a spectrometer or any system for measuring spectral intensities. The system may include the light source 2902 that projects light through slit 2926, the collimator lens 2904, sample cell 2906, and diversifier 2920 to detector 2922. The divider 2908, which may be optional, may allow for additional wavelengths and/or improved light source control based on external light source 2912. Light divider 2914 may redirect a portion of the light from the sample cell 2906 to the external spectrometer 2918 for measurement.

The light source 2902 may be any source of light including, but not limited to, a laser, LED, or the like. The light source 2902 may be controlled by the data processing unit 2924. In one example, the data processing unit 2924 may control the light source 2902 to project any number of wavelengths using selected currents. By controlling the light source 2902, the data processing unit 2924 may correlate wavelengths to scattered patterns detected by the detector 2922.

The slit 2926 may comprise any material and may include one or more holes or slits to assist in collimation of the light source 2902. The slit 2926 may be optional.

The collimator lens 2904 may be any lens capable of collimating and/or focusing light from the light source 2902 to the sample cell 2906. The system may include a light source 2902 that projects light through a collimator lens 2904 (e.g., a collimator) and then through sample cell 2906. The sample cell 2906 may be a cuvette or any object for retaining a sample. When measuring the impact of the diversifier 2920, the sample cell 2906 may not contain a biological sample. In some embodiments, the sample cell 2906 contains water, VTM, or the like. In some embodiments, the sample cell 2906 may not contain any material (e.g., fluid or otherwise) when measuring the speckle. In various embodiments, the sample cell 2906 is not there, meaning that there is no object in that part of the light path (e.g., the cuvette is removed or not inserted in the light path).

The light along the light path may pass through the sample cell 2906 (or the space for sample cell 2906) and may be divided by a light divider 2914. Light from light divider 2914 may divert a portion of the light received from the sample cell 2906 to lens 2916 to the external spectrometer 2918 (via fiber or secondary light path F2). The external spectrometer 2918 may be, in some embodiments, only a second detector. Lens 2916 may focus or otherwise collimate the light. In various embodiments, the external spectrometer 2918 may measure spectral intensities right before light passes through the diversifier 2920. As such, the data processing unit 2924 may assess measurements from the detector 2922 (taken of the scattered light of the diversifier 2920) and the measurements from the external spectrometer 2918. The assessment may be further analyzed by considering the wavelengths and currents provided by the light source 2902 and/or the external light source 2912.

Data processing unit 2924 may take the measurements to assist in generating a filter based on the diversifier in order to reduce noise. In one example, this may be accomplished by simultaneously measuring the diversifier (e.g., a speckle field or diffuser) with a 2D pixel array and measuring a spectrum while modulating the spectrum of the light source.

The data processing unit 2924 may control the light source 2902 to select wavelengths for measurements by detector 2922. In some embodiments, the data processing unit 2924 does not control the light source 2902 but rather receives wavelength information of light generated by the light source 2902 (e.g., the light source 2902 provides wavelength and any other information to the data processing unit 2924). In some embodiments, the data processing unit 2924 may also control the external light source 2912 to select wavelengths for measurements by detector 2922. In some embodiments, the data processing unit 2924 does not control the external light source 2912 but rather receives wavelength information of light generated by the external light source 2912 (e.g., the light source external 2912 provides wavelength and any other information to the data processing unit 2924).

The data processing unit 2924 may also receive spectral measurements from the detector 2922. For example, the detector 2922 may receive information or make measurements based on light received from the light source 2902 (e.g., after being projected through sample cell 2906 and the diversifier 2920). The data processing unit 2924 may also receive spectral measurements from the detector of the external spectrometer 2918. For example, the detector may receive information or make measurements based on light received from the light source 2902 (e.g., after being projected through sample cell 2906 and partially diverted by the light divider 2914).

The data processing unit 2924 and/or other digital devices may utilize the information regarding wavelength transmitted over the light path, the spectral intensities detected by the external spectrometer detector (before the light passes through the diversifier 2920), and the spectral intensities detected by the detector 2922 (after the light passes through the diversifier 2920) to identify patterns of light scattering caused by the diversifier 2920.

The data processing unit 2924 may associate detected spectral patterns of scattering caused by the diversifier 2920 with current and wavelength. In various embodiments, the data processing unit 2924 and/or other digital devices may utilize a DNN or other pattern recognition approaches to detect patterns and associate the patterns with the wavelengths and current of the light source generated the light provided to the light path.

The data processing unit 2924 may generate filtering information (e.g., a function to characterize diffusion or speckle based on wavelength and current). Using this information, the data processing unit 2924, other processors (e.g., in the cloud), and/or AI systems may generate filters to remove noise and significantly improve the signal to noise ratio (SNR).

The diversifier 2920 may be a speckle pattern, diffuser, series of vortexes, and/or the like. In various embodiments, the diversifier 2920 is any material with occlusions or obstructions that cause scattering of the light provided by the light source. The scattered, incoherent light may be detected by the detector 2922 for pattern recognition and filter information generation for improvement of signal detection.

In some embodiments, a well-defined and controlled light source (e.g., capable of providing any number of wavelengths over different currents), such as external light source 2912 may be utilized to assist in measuring the scattered light caused by the diversifier. The external light source 2912 may provide light at known wavelengths via fiber (or light path) F1, through lens 2910 to divider 2908. The divider 2908 may project the light from the external light source 2912 through sample cell 2906 to the light divider 2914 and diversifier 2920. The external light source 2912 may be used to assist in measuring any number of systems using different diversifiers which may allow for simplified association of patterns of scattered spectral intensities caused by interaction with the diversifier. It will be appreciated that the divider 2908, lens 2910, and external light source 2912 may be optional.

Spectra may be recreated from speckle diffusers and/or vortex matrices in many different ways. In some embodiments, light from a light source (in a spectrometer) is measured (e.g., by a camera) after the light is transmitted through a medium with different absorption features and subsequently transmitted through a particular diffuser containing a speckle pattern. The diffuser creates a wavelength dependent speckle structure in the images captured by the CMOS camera. The captured images may be used to train a Deep Neural Network (DNN) to reconstruct the original spectra. The use of a CMOS camera and speckle diffuser allows for a much more compact and cheaper spectroscopy system. With comparable accuracy, this system may allow inexpensive spectrometers to be integrated into a wide variety of applications.

In another example, a light source 2902 (e.g., a SuperContinuum light source) of a spectrometer may transmit broadband light through a sample cell 2906, through a speckle diversifier 2920, and onto a detector 2922 (e.g., a CMOS camera). The light from the light source 2902 may impinge on a beam cube (e.g., divider 2908) and splits the light in two different directions, to the right and downward. The light from the right path is passed through a cuvette (e.g., sample cell 2906) that can contain a sample before going through a polarizing filter and then impinging on another beam cube (light divider 2914). This splits the beam into two paths again, one path going straight through to the external spectrometer 2918. The other path sends the light through the speckle diffuser filter (e.g., diversifier 2920). Before the CMOS camera (e.g., detector 2922), there may be a microscope filter (e.g., 60X Microscope Objective) between the diversifier 2920 and the detector 2922 (not depicted in FIG. 29) to zoom in on the speckle of the diversifier 2920.

The data from this example may be used to train a DNN to reconstruct spectra. For example, the data from this example may be split into input and output sections. The inputs may include speckle diffuser images captured by the CMOS camera (e.g., detector 2922). The output may include simultaneously captured spectra of the light transmitted through the sample (sample cell 2906). In one example, this may be done using an Avantes Spectrometer and saving the spectra as a Numpy array. This spectrum may be used as the true spectrum in the DNN regression analysis after the training.

A basis set may be created to span the possible output wavelengths of light from the Supercontinuum light source (e.g., light source 2902). This set may be generated by sweeping light (e.g., from 450 nm to 1000 nm with a step size of 5 nm). The use of a basis set allows for a much larger dataset to be generated from a small amount of data. Feature changes may be applied that affect the speckle diffuser image by applying the weight of each wavelength of the spectrum to the image.

Subsequently, the speckle diffuser images may be further processed to highlight the differences between feature changes in the spectra. To do this, an image and spectra may be generated without any absorption features. This is done to show only the feature changes in the final output image for the Neural Network training. Each image of the dataset may be loaded in one-by-one and first averaged to remove the mean of the image from itself. This is a standard statistics procedure to ensure that the dataset is zero mean with a standard deviation of one. The zero mean image may be then normalized by the absolute value of the total sum of intensity of the image. This helps to remove any bias in the dataset and ensures a fair comparison between the data points. Subsequently, the “no absorption” image is subtracted from the dataset image to leave only the feature changes that were applied to the spectra. Note that the spectrum is not changed in this step. After this processing, the dataset may be passed through another script that links the images and spectra for use in the Neural Network training.

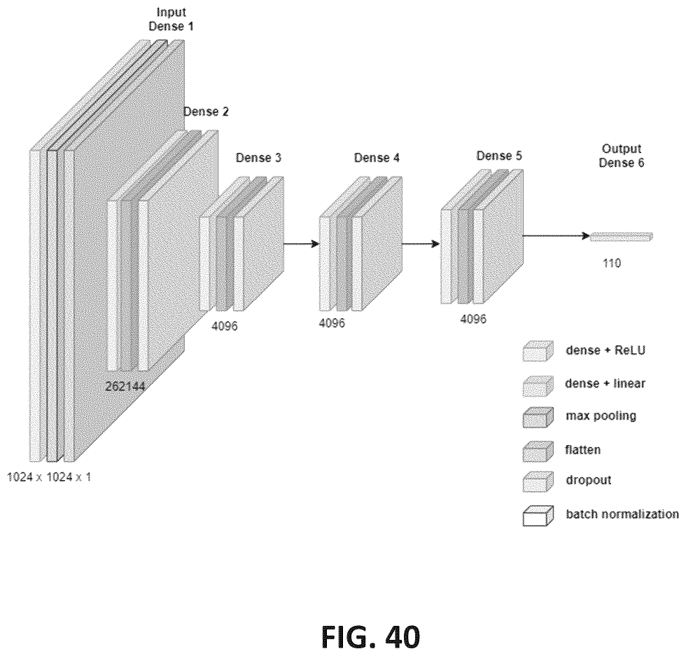

A Regression based Deep Neural Network may be used to achieve spectral reconstruction. Regression is used in this example to predict an array of floating point values that correspond to the original spectrum. The model may be composed of multiple Dense Layers used in the Input Layer, Hidden Layers, and Output Layer. FIG. 40 depicts an example deep neural network and example configuration in some embodiments. Between each Dense Layer are Batch Regularization and Dropout Layers to help the network generalize.

In previous testing, the configuration of the deep neural network depicted in FIG. 40 recreated the spectra with a mean error of 0.0322 and an error standard deviation of 0.0328. Error may be calculated by dividing the total sum of the absolute value of the difference between the predicted spectra and actual spectra by the absolute value of the sum of the actual spectrum.

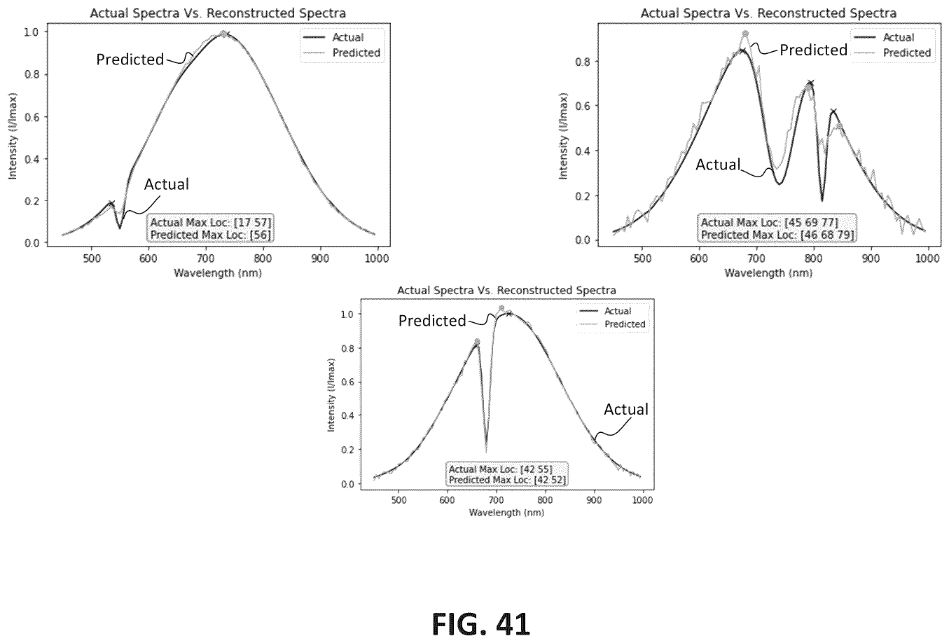

FIG. 41 includes comparisons of actual spectra plotted against reconstructed spectra in testing in some embodiments.

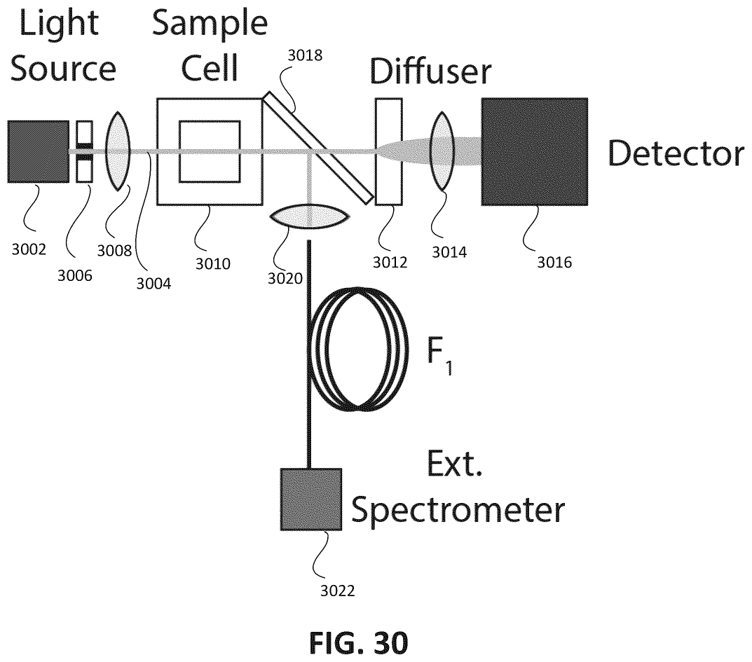

FIG. 30 depicts a system for spectral reconstruction based on scattered light caused by a diversifier but without an external light source in some embodiments. The system depicted in FIG. 30 may be similar to that of FIG. 29 but without the external light source 2912, divider 2908, and lens 2910. Similar to FIG. 29, the system of FIG. 30 depicts a light source 3002, light path 3004, a slit 3006, lens 3008, sample cell 3010, diffuser 3012, lens 3014, detector 3016, divider 3018, lens 3020, and external spectrometer 3022.

The light source 3002 may be similar to the light source 2902 and may project light along light path 3004 to the detector 3016. Although not depicted, a processor (e.g., data processing unit) may control the light source to control current and wavelength for scatter pattern testing of the diffuser 3012. The slit 3006 may be similar to slit 2926.

Further, the lens 3008, sample cell 3010, divider 3018, detector 3016, lens 3020, and external spectrometer 3022 may be similar to collimator lens 2904, sample cell 2906, light divider 2914, detector 2922, lens 2916, and external spectrometer 2918, respectively.

In this example, the light source 3002 may provide light at specific frequencies when needed for testing.

The diffuser 3012 may be any diversifier. In one example, the diffuser 3012 may comprise crushed glass. It will be appreciated that the diffuser 3012 and/or diversifier 2920 may comprise any material(s) that allow for light to pass but also includes speckle, occlusions, or obstacles to scatter light. In some embodiments, the diffuser 3012 may be a speckle that is reproduceable (e.g., by tape, reproduced film, 3D hologram, or the like).

The lens 3014 may collimate or focus light received from the diffuser (which may spread as a result of the scattering) before the light is received by the detector 3016. In some embodiments, the lens 3014 may project a portion of the light that passes through the diffuser 3012. For example, one or more lenses may direct light that is scattered by one or more specific occlusions of the diffuser 3012 to the detector 3016. In some embodiments, one or more lenses may focus scattered light from a subset of occlusion and/or vortexes of the diffuser 3012 to the detector. In some embodiments, light from the areas of the diffuser 3012 that is not being projected through the subset of occlusions may be ignored, blocked, or otherwise disregarded for pattern recognition and/or signal detection improvement.

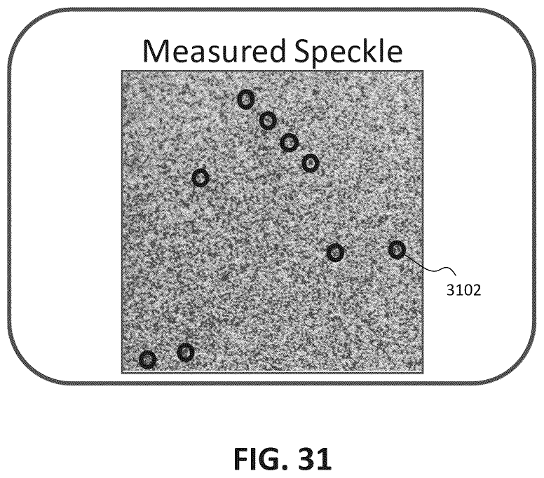

FIGS. 31-33 depict different types of diversifiers. FIG. 31 is an example of a speckle pattern in some embodiments. In one example, the speckle pattern is composed of bright and dark speckle that create interference of spatially coherent light. A diversifier may be at least partially transparent and include speckle similar to the speckle pattern depicted. Light scattered by a sample in the sample cell is spatially incoherent and scatters light into the dark speckle cores. The spectral reconstruction signal contrast can be enhanced by limiting the reconstruction to pixels in the dark spectral cores (e.g., dark spectral core 3102 and those cores that are circled).

In various embodiments, the material with the speckle pattern is transparent to allow light to pass the material although with the scattering effect caused by the speckle. A detector (e.g., detector 2922 depicted in FIG. 29) may receive the light after being scattered by the speckle.

Some of the speckle may create greater scattering effects than other speckle. In some embodiments, after light is passed through the speckle pattern and the speckle is measured (e.g., as a function of current and wavelength), specific aspects or occlusions of the speckle (e.g., occlusion or dark spectral core 3102) may provide scattering that is associated with wavelength and current. Other aspects or occlusions of the speckle may not provide sufficient patterns or information to associate with the wavelength of the projected light and, as such, may be disregarded. As such, filter information may be generated that utilizes some but not all of the speckle for noise reduction.

The speckle pattern and/or material includes the speckle pattern may be created in any number of ways. In some embodiments, tape (e.g., scotch tape) may be used to collect debris to create the speckle effect. In another example, glass with an etched image or containing a hologram may further contain marks or material to create the speckle pattern.

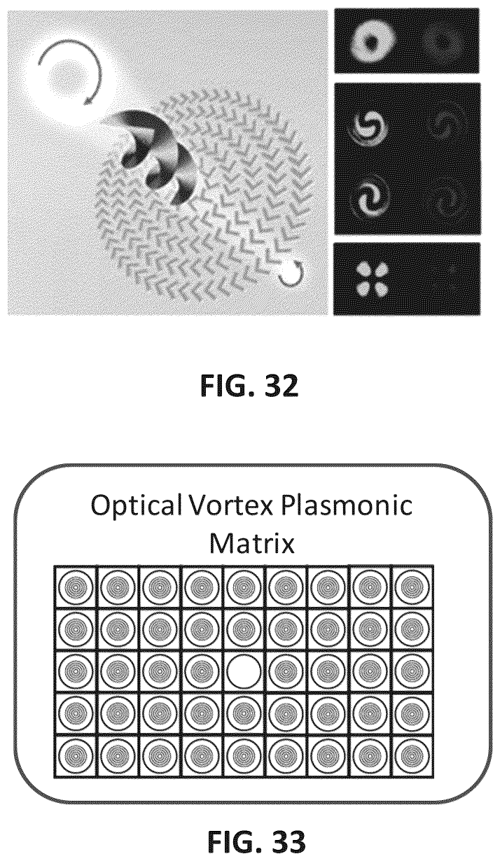

In some embodiments, instead of speckle, an optical vortex meta-surface may be utilized. FIG. 32 depicts an example optical vortex meta-surface. In some embodiments, the diversifier (e.g., diversifier 2920) may be an optical vortex meta-surface.

Like dark speckle cores, optical vortices form from the destructive interference of coherent light. An optical vortex plasmonic matrix may be a two-dimensional array of subwavelength plasmonic metasurfaces and may be used to create a uniform vortex lattice. Similar to the speckle cores, the spectral reconstruction signal contrast can be enhanced by limiting the reconstruction to pixels in the dark vortex cores.

As discussed herein, when the light passes through a scattering medium containing particles larger than the wavelength, light is scattered. The most intense scattering usually occurs in the forward direction. Light scattered along the optical axis is often difficult to distinguish from the superimposed unscattered laser beam, especially when there is a dilute concentration of weak scatterers. This scattered light may interfere with the light of the principal beam and, as a result, speckle (i.e., noise) may be formed.

In some cases, particular wavelengths may be absorbed by the scattering media. This occurs because the light at those particular wavelengths excite the rotational or vibrational state of the molecules in the media. Therefore, the chemical makeup of an absorbing media may be based on the spectral absorption signature that is present. If the medium is weakly scattering (i.e., there are few scatterers), the absorption signature may be overwhelmed by the strong on-axis unscattered light source. Therefore, in order to optimize the characterization of the scattering molecules, a light suppression technique may be utilized to attenuate the strong on-axis source while leaving the weaker scattered signal intact.

An optical vortex is a dark null of destructive interference that occurs at a spiral phase dislocation in a beam of spatially coherent light. The phase of a transmitted light beam may be twisted and light from opposite sides of the mask may coherently destructively interfere to form a dark null in the transmitted intensity pattern, much like the eye of a hurricane.

The vortex may assist in creating destructive interference of the light source, thereby enabling improved sensitivity of fainter signals.

FIG. 33 depicts an optical vortex plasmonic matrix that may be utilized rather than the diffuser, speckle pattern, or optical vortex meta-surface. The optical vortex plasmonic matrix may include any number of optical vortex meta-surfaces. Each of the optical vortex meta-surfaces may be different from each other. For example, each of the optical vortex meta-surface of the optical vortex plasmonic matrix may have different properties to allow for destructive interference of coherent wavelengths of light, thereby allowing for improved sensitivity of fainter signals of incoherent light.

It will be appreciated that different wavelengths projected by the light source may be affected differently by one or more vortexes of the optical vortex plasmonic matrix. One or more different vortexes of the optical vortex plasmonic matrix may produce patterns and signals. A detector may assess all or only specific optical vortexes of the optical vortex plasmonic matrix to identify patterns associated with different currents and/or wavelengths to enable boosting of signals of interest and spectral reconstruction to eliminate noise.

Each of the vortexes of the optical vortex plasmonic matrix may perform similarly to the function of the vortex of the vortex mask 1540 discussed regarding FIG. 15. For example, the vortex may be an optical vortex coronagraph.

An example optical vortex coronagraph uses a helical phase of the form eiϕ, with ϕ=lθ, where l is the topological charge and θ is the focal plane azimuthal coordinate. In optical systems, vortices manifest themselves as dark donut of destructive interference that occur at phase singularities. For example, E(ρ, ϕ, z, t)=A(ρ, z) exp(ilθ) exp(iωt−ikz) where (ρ, ϕ, z) are cylindrical coordinates, A(ρ, z) is a circularly symmetric amplitude function and k=2π/λ is the wavenumber of a monochromatic field of wavelength λ.

In some embodiments, the optical vortex coronagraph may utilize a rotationally symmetric half wave plate which can generate an azimuthal phase spiral reaching an even multiple of 2 pi radian.

Although the optical vortex plasmonic matrix of FIG. 33 depicts a 5×9 array of vortexes (with a missing vortex in the middle), it will be appreciated that there may be any number of vortexes in any orientation.

In some embodiments, the optical vortex plasmonic matrix of FIG. 33 includes a non-integer vortex array. A non-integer vortex array may have a random, non-integer topological charge, lₘₙ for a given desired wavelength λ₀ given by:

lₘₙ=l₀+Δlₘₙ

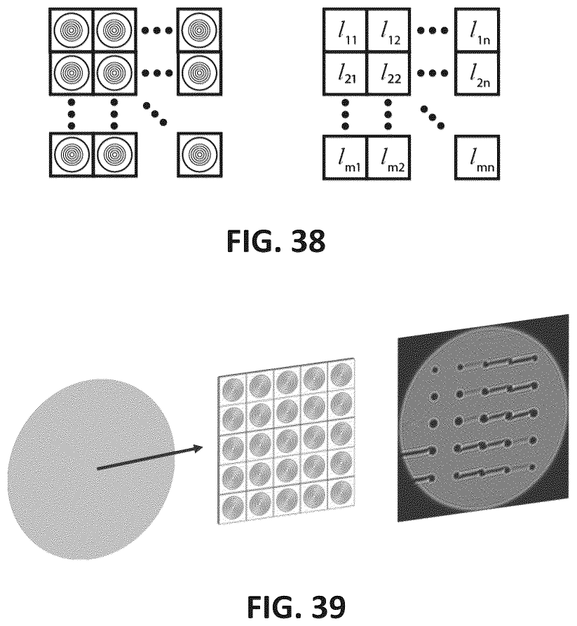

FIG. 38 depicts an example mapping of an optical vortex plasmonic array to a non-integer topological charge lₘₙ.

Light transmitted through the non-integer vortex array waveplate may gain spatially distributed chromatic dispersion, with wavelength dependent phase fronts for each vortex given by:

$$Φ ( x , y ) = {l}_{mn} \cdot ( \frac{λ}{{λ}_{0}} ) \cdot {θ}_{mn} ( x , y )$$

where λ₀ is the design wavelength that corresponds to the topological charge l₀ and θ(x, y) is the azimuthal polar coordinate centered on each vortex in the array. FIG. 39 is an example graphic of light shining on a non-integer vortex array waveplate and an example resulting image in some embodiments.

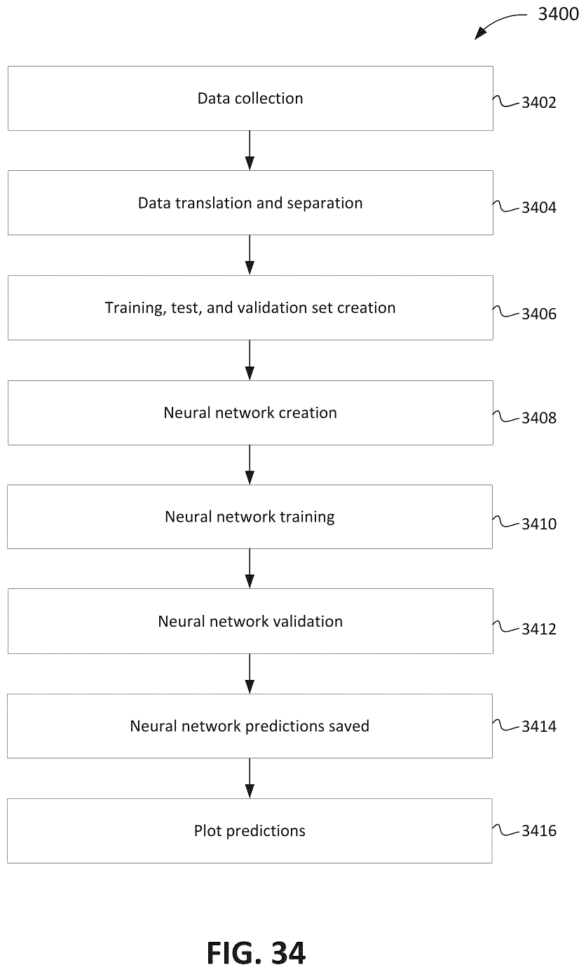

FIG. 34 is a flowchart for creating filtering for spectral reconstruction in some embodiments. As discussed herein, the speckle pattern may be measured as a function of spectrum and current. The measurement(s) may then be modeled to create a filter to improve accuracy of spectral measurements when passed through a sample (e.g., a biological sample) and the diffuser or speckle pattern. In various embodiments, a processor may receive simultaneous or near simultaneous measurements of current and wavelength as the light passes through the speckle pattern. The processor may then determine a variation of speckle pattern as a function of wavelength.

The speckle patterns generated after transmitting light through the diffuser or speckle pattern may be different for each wavelength, with the presence of inherent noise (e.g., caused by instruments and/or environmental factors). In various embodiments, neural networks, such as a deep neural network, may be used for classification. Through training, the DNN may learn to reject variations in the speckle patterns which do not correspond to wavelength.

In step 3402, a spectral reconstruction device performs data collection. A spectral reconstruction device may be any digital device. In one example, the spectral reconstruction device is a cloud-based system configured to collect information, translate/separate data, train/test/and validate set creation, utilize a neural network for predictions, and plot predictions. The spectral reconstruction device may provide plot predictions for any number of spectrometers. In some embodiments, the spectral reconstruction device is local. In various embodiments, different functions of the spectral reconstruction device may be performed locally while other functions may be performed in the cloud.

In various embodiments, the spectral reconstruction device communicates with a spectrometer (e.g., the spectrometer discussed in FIG. 29 and the data processing unit 2924 may be the spectral reconstruction device).

Data from a detector of the spectrometer may be stored in any number of ways. In one example, the spectral reconstruction device creates a Pandas DataFrame to store the data received from a laser diode controller (e.g., a controller that controls a light source of the spectrometer) and detector of the spectrometer. The spectral reconstruction device may include a resource manager to speak with the laser diode controller (e.g., Using PyVisa and SCPI commands from Thorlabs). Following this, the spectral reconstruction device may create variables to set parameters for the laser diode controller and detector of the spectrometer. This may include, for example, the IntegrationTime (e.g., 30 μs), NumberOfAverages (e.g., 9), Laser1Temperature bounds and tolerance (e.g., 18° C.), and Laser1Current bounds and tolerance (e.g., 28 mA-34.3 mA, 0.1 mA). From here, contact with the laser diode controller and detector of the spectrometer may be initiated, data may be gathered on the wavelengths of the detector, and the data passed through configuration parameters.

In some embodiments, the data collection is enclosed in a loop that starts at the lower bound of the current and iterates to the upper bounds. Temperature bounds may be provided to the controller and then read back to ensure correctness. The same may be done for the current. When both bounds are within their set tolerances, the spectrometer may capture spectral data. The array is then interpolated over the set wavelengths of the spectrometer. The current, temperature, spectral data, and wavelengths may be stored (e.g., saved to a new row of a Pandas DataFrame). Once the iterations are complete, the data is saved (e.g., DataFrame is then saved out to a CSV file), and the Laser Diode is turned off.

In step 3404, data is translated and separated. For example, the spectral data may be placed in its own DataFrame and split up into cells (e.g., into 2050 cells). The first and last cells may be deleted as they may contain no information. Individual cells may be processed to turn them from string objects into float objects for later use in arrays (e.g., NumPy arrays). The original DataFrame may be referenced again to extract the current data and stored in its own DataFrame. The current data and spectral data may be concatenated into one structure for use in the neural network. The combined DataFrame and spectral data DataFrame may be saved (e.g., into Pickle files) for later use.

In step 3406, the data set is created. For example, current data and spectral data may be loaded into a DataFrame. The first column that correlates to the current data may be translated to a Numpy Array. The same may be done for the spectral data in its own Numpy Array. These arrays may be then passed into a function that splits the data into training and test sets. The test sets may then be split again into test sets and validation sets.

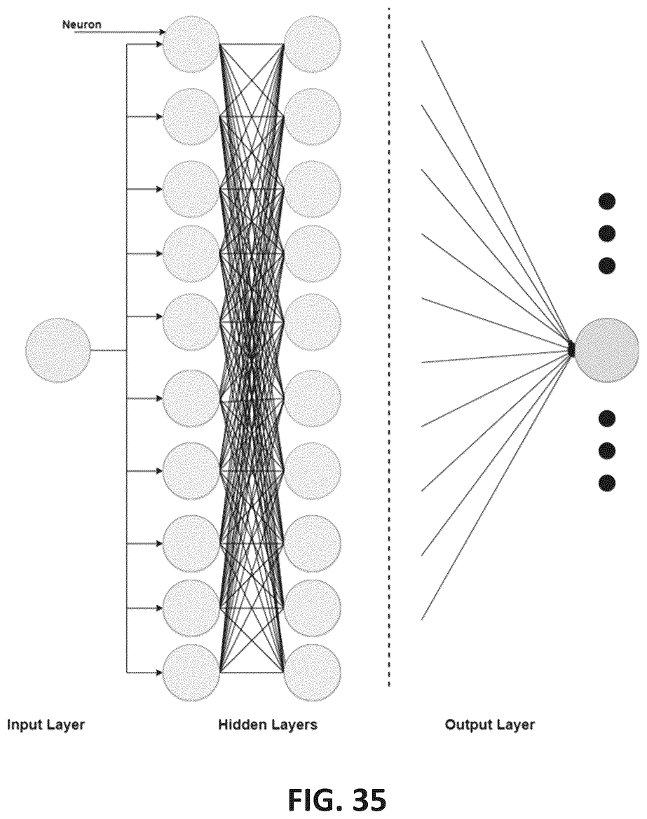

In step 3408, individual layers of the neural network are created. In some embodiments, the neural network consists of an input layer of one neuron that matches the dimension of the current data array. In some embodiments, there may be a number of fully connected dense layers (e.g., 9 fully connected dense layers) each with a number of neurons (e.g., 100 neurons) with the ReLu activation function. The output layer may be another dense layer but with a number of neurons (e.g., 2048 neurons) that correspond to the points (e.g., 2048 points) in the output spectral data. FIG. 35 depicts an example architecture of the neural network.

In some embodiments, to improve computation and structural efficiency, these layers may be added to a Tensorflow model. An Adam optimizer (e.g., with a learning rate of 0.01) may be created and added to a compilation of the model. The loss function used may be, for example, mean squared error with the accuracy model being tracked. The model may then fit on the training set (e.g., over 250 epochs with a batch size of 20).

In step 3410 many different wavelengths are repeated projected through the diversifier and scattering patterns detected to confirm and train the neural network. As discussed herein, spectral patterns of scattered light may be reproduceable for identifying wavelengths projected by the light source. Light at specific wavelengths may be projected for specific durations of time (e.g., some longer durations and other shorter durations), and the spectral information provided to the neural network for testing with known data and training.

In step 3412, the neural network results are validated against the validation set that was created earlier (e.g., against known wavelengths as well as the patterns that are expected based on the neural network).

In step 3414, predictions of the neural networks are saved and used to generate filtering information to allow for spectral reconstruction. Each different spectrometer may have different filtering information that has been tuned or configured based on noise inherent in that spectrometer's components, light sources, detectors, and/or light path. The filtering information may be stored in the cloud for each different spectrometer. In some embodiments, the filtering information associated with a particular spectrometer may be stored at a digital device proximate to the spectrometer or by the spectrometer (assuming the spectrometer has a processor and memory for utilizing the filtering information).

In step 3416, predictions are plotted, and noise reduced or eliminated by using the filtering information during testing of user's samples. The predictions may then be saved as a NumPy array for further analysis and graphing.

Training may be validated against the validation set that was created earlier. Once scattering patterns are recognized and associated with current and wavelength (e.g., using the neural network discussed herein), the associations may be tested with known wavelengths and expected scattering patterns in step.

Once training is complete, the test set may be used to test the model's completed predictions.

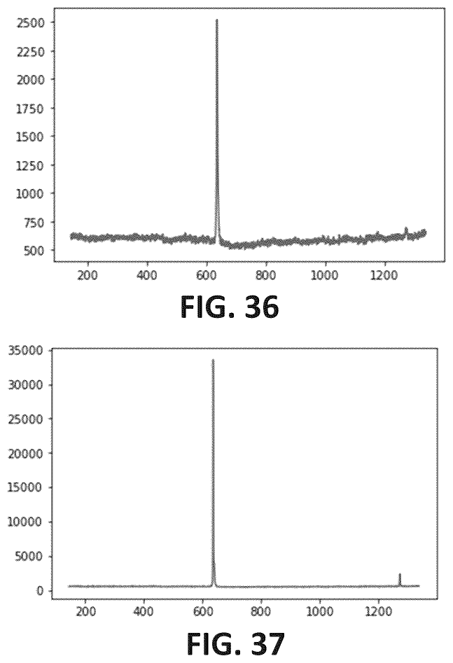

FIG. 36 is an example spectra taken from the data collected from the spectrometer. In this example, there was a low current input which is why the 635 nm peak only reaches ˜2500 counts.

FIG. 37 is a spectra that was reconstructed by the neural network. While the spectra reaches a much higher peak power, the signal-to-noise ratio is about 10 times greater than the initial data that it was given. It will be appreciated that further refinements may further improve the signal-to-noise ratio.

Once filtering information is established for a spectrometer, the external spectrometer and/or external light sources may be removed.

Any number of spectrometers may be utilized to assess spectral patterns associated with a pathogen (e.g., covid-19). In various embodiments, as discussed herein, the spectrometer(s) and related filtering information may be used to eliminate noise. Vortexes may further be used to identify signals (e.g., spectral scattering caused by the pathogen) that may be otherwise too faint to detect (e.g., because the pathogen being tested is very small). The signature of thumbprint of the spectral signature may be detected and trained such that the pathogen may be confidently and accurately detected in future samples.

Steps for generating a pathogen signature may be similar to that discussed regarding FIG. 22. Using a spectrometer with a diversifier (e.g., speckle pattern, diffuser, vortex plasmonic array, and/or the like) as well as the filtering information to eliminate/reduce noise, samples containing the pathogen may be repeated tested and spectral intensities detected. Any number of spectrometers may be used (e.g., each with their own diversifier and filtering information). The filtering information may be used to reconstruct the spectral signal of the wavelengths of light provided by the light source so to isolate those signals caused by the presence of the pathogen in the sample. By using the system described with the diversifier and filtering information, signal qualities related to the pathogen may be more apparent and signature acquired may be more accurately defined and detected.

In various embodiments, a statistical process and/or machine learning process may be utilized to identify patterns of spectral scattering/absorption caused by the presence of a known pathogen in a sample. Concentration of pathogen and duration of testing may be controlled to assist in pattern recognition for pathogen signature identification. In various embodiments, a CNN (e.g., DNN) may be trained to identify and recognize patterns related to pathogen presence in the sample based on repeated testing of known concentrations of pathogens in a sample, known wavelengths, and the filtering information.

In various embodiments, a plurality of spectrometers (each with their own diversifiers) may test any number of samples with a known pathogen. Results of the tests may be provided to a central system on a network (e.g., in the cloud) which may then utilize results (and/or filtering information associated with each spectrogram) for pathogen signature detection. The centralized system may be utilized to leverage decentralized testing of any number of pathogens over any number of remote spectrometers. By centralizing the system on a network, the system may be able to detect patterns, define signatures, improve signatures, and provide consistent pathogen results to any number of remote operators and/or patients.

It will be appreciated that the centralized system may allow for results of tests of samples to be confidentially handled. For example, results and testing information may be securely (e.g., via encryption) stored and communication handled to ensure effective, confidential processes are conducted for information security and privacy in accordance with state, national, and international standards. This may also ensure processes are in place to communicate results to those who have a right to the information.

Once signatures are created, the centralized system may validate and test pathogen signatures against samples of known pathogens to confirm accuracy and robustness. It will be appreciated that the centralized system may test new samples with the signatures for presence of the particular pathogen and then utilize information of the sample, results, and any other information to update signatures or create new signatures as pathogens evolve or separate into new strains.

Further, once signatures are created, the centralized system may provide pathogen signatures to other centralized systems on a network and/or spectrometers for use in pathogen detection in new samples. Alternately, the centralized system may maintain pathogen signatures and compare sample results from any number of spectrometers to the signatures for centralized pathogen detection. In various embodiments, the centralized system may utilize probabilistic methods to determine a likelihood of match between the results from the spectrometers (e.g., the spectrometers including diversifiers and the results being adjusted with the filtering information) and the pathogen signature. In some embodiments, the centralized system may utilize a CNN, DNN, boosted decision trees, or the like to determine a likelihood of match between the results from the spectrometers and the pathogen signature.

In some embodiments, the centralized system may test results from a spectrometer against any number of pathogen signatures. In one example, the centralized system may receive results from a spectrometer with a diversifier and may compare or analyze the results against a set of two or more pathogen signatures to detect the presence of one or more pathogens in the sample.

In various embodiments, a pathogen signature may be created for a class or group of pathogens (e.g., five different variants of E. coli). If a sample is determined to have pathogens based on a comparison or analysis using the pathogen signature (a “group pathogen signature”), then indications of infection may be provided to the user or operator. In some embodiments, after a sample is determined to have pathogens, further pathogen signatures may be used to identify which pathogen(s) of the class or group of pathogens is present in the sample.

As discussed herein, measurements conducted by a spectrometer with a diversifier may be performed over any duration of time (e.g., while the light source is projecting light over a period of time). In some embodiments, the light source may direct light to a portion of the cuvette containing the sample and/or to a particular portion of the diversifier. In some embodiments, the detector or lenses may be configured to analyze only a portion of the light being transmitted through the diversifier for scattering pattern detection, testing, validation, and/or biological sample spectral detection.

FIG. 42 depicts a block diagram of an example digital device 4200 according to some embodiments. The digital device 4200 is shown in the form of a general-purpose computing device. The digital device 4200 includes at least one processor 4202, RAM 4204, communication interface 4206, input/output device 4208, storage 4210, and a system bus 4212 that couples various system components including storage 4210 to the at least one processor 4202. A system, such as a computing system, may be or include one or more of the digital device 4200.

System bus 4212 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

The digital device 4200 typically includes a variety of computer system readable media, such as computer system readable storage media. Such media may be any available media that is accessible by any of the systems described herein and it includes both volatile and nonvolatile media, removable and non-removable media.

In some embodiments, the at least one processor 4202 is configured to execute executable instructions (for example, programs). In some embodiments, the at least one processor 4202 comprises circuitry or any processor capable of processing the executable instructions.

In some embodiments, RAM 4204 stores programs and/or data. In various embodiments, working data is stored within RAM 4204. The data within RAM 4204 may be cleared or ultimately transferred to storage 4210, such as prior to reset and/or powering down the digital device 4200.

In some embodiments, the digital device 4200 is coupled to a network, such as the communication network 108, via communication interface 4206. Still yet, the light intensity measuring apparatus 102, the foodborne pathogen detection system 104, the food processing apparatus 106, and the computing device 110 can communicate with one or more networks such as a local area network (LAN), a general wide area network (WAN), and/or a public network (for example, the Internet).

In some embodiments, input/output device 4208 is any device that inputs data (for example, mouse, keyboard, stylus, sensors, etc.) or outputs data (for example, speaker, display, virtual reality headset).

In some embodiments, storage 4210 can include computer system readable media in the form of non-volatile memory, such as read only memory (ROM), programmable read only memory (PROM), solid-state drives (SSD), flash memory, and/or cache memory. Storage 4210 may further include other removable/non-removable, volatile/non-volatile computer system storage media. By way of example only, storage 4210 can be provided for reading from and writing to a non-removable, non-volatile magnetic media. The storage 4210 may include a non-transitory computer-readable medium, or multiple non-transitory computer-readable media, which stores programs or applications for performing functions such as those described herein with reference to, for example, FIG. 3A and FIG. 3B. Although not shown, a magnetic disk drive for reading from and writing to a removable, non-volatile magnetic disk (for example, a “floppy disk”), and an optical disk drive for reading from or writing to a removable, non-volatile optical disk such as a CDROM, DVD-ROM or other optical media can be provided. In such instances, each can be connected to system bus 4212 by one or more data media interfaces. As will be further depicted and described below, storage 4210 may include at least one program product having a set (for example, at least one) of program modules that are configured to carry out the functions of embodiments of the invention. In some embodiments, RAM 4204 is found within storage 4210.

Programs/utilities, having a set (at least one) of program modules, such as foodborne pathogen detection system 104, may be stored in storage 4210 by way of example, and not limitation, as well as an operating system, one or more application programs, other program modules, and program data. Each of the operating system, one or more application programs, other program modules, and program data or some combination thereof, may include an implementation of a networking environment. Program modules generally carry out the functions and/or methodologies of embodiments of the invention as described herein.

It should be understood that although not shown, other hardware and/or software components could be used in conjunction with the digital device 4200. Examples include, but are not limited to microcode, device drivers, redundant processing units, and external disk drive arrays, RAID systems, tape drives, and data archival storage systems, etc.

Exemplary embodiments are described herein in detail with reference to the accompanying drawings. However, the present disclosure can be implemented in various manners, and thus should not be construed to be limited to the embodiments disclosed herein. On the contrary, those embodiments are provided for the thorough and complete understanding of the present disclosure, and completely conveying the scope of the present disclosure.

It will be appreciated that aspects of one or more embodiments may be embodied as a system, method, or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), a solid state drive (SSD), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program or data for use by or in connection with an instruction execution system, apparatus, or device.

A transitory computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++, Python, or the like and conventional procedural programming languages, such as the “C” programming language or similar programming languages. The computer program code may execute entirely on any of the systems described herein or on any combination of the systems described herein.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

While specific examples are described above for illustrative purposes, various equivalent modifications are possible. For example, while processes or blocks are presented in a given order, alternative implementations may perform routines having steps, or employ systems having blocks, in a different order, and some processes or blocks may be deleted, moved, added, subdivided, combined, and/or modified to provide alternative or sub-combinations. Each of these processes or blocks may be implemented in a variety of different ways. Also, while processes or blocks are at times shown as being performed in series, these processes or blocks may instead be performed or implemented concurrently or in parallel or may be performed at different times. Further any specific numbers noted herein are only examples: alternative implementations may employ differing values or ranges.

Throughout this specification, plural instances may implement components, operations, or structures described as a single instance. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. Furthermore, any specific numbers noted herein are only examples: alternative implementations may employ differing values or ranges.

Components may be described or illustrated as contained within or connected with other components. Such descriptions or illustrations are examples only, and other configurations may achieve the same or similar functionality. Components may be described or illustrated as “coupled”, “couplable”, “operably coupled”, “communicably coupled” and the like to other components. Such description or illustration should be understood as indicating that such components may cooperate or interact with each other, and may be in direct or indirect physical, electrical, or communicative contact with each other.

Components may be described or illustrated as “configured to”, “adapted to”, “operative to”, “configurable to”, “adaptable to”, “operable to” and the like. Such description or illustration should be understood to encompass components both in an active state and in an inactive or standby state unless required otherwise by context.

The use of “or” in this disclosure is not intended to be understood as an exclusive “or.” Rather, “or” is to be understood as including “and/or.” For example, the phrase “providing products or services” is intended to be understood as having several meanings: “providing products,” “providing services”, and “providing products and services.”

It may be apparent that various modifications may be made, and other embodiments may be used without departing from the broader scope of the discussion herein. For example, while the foodborne pathogen detection system 104 is described as providing reports via websites, the foodborne pathogen detection system 104 may provide reports via applications executing on computing devices, such as apps executing on phones and/or mobile devices and/or native applications executing on laptop or desktop computers.

As another example, the foodborne pathogen detection system 104 may detect pathogens and/or contaminants in water, such as water provided by community or municipal water systems, private wells, and/or bottled water producers. For example, the foodborne pathogen detection system 104 may utilize the techniques described herein to test for microorganisms such as cryptosporidium, giardia lamblia, legionella, and enteric viruses, inorganic chemicals such as cadmium, chromium, mercury, selenium, organic chemicals such as benzene, glyphosate, vinyl chloride, disinfectants, disinfection byproducts, and/or radionuclides. It will be understood that the foodborne pathogen detection system 104 may utilize the techniques described herein to detect regulated pathogens and/or contaminants according to standards set by government agencies such as the U.S. Environmental Protection Agency as well as pathogens and/or contaminants unregulated by such government agencies.

Therefore, these and other variations upon the example embodiments are intended to be covered by the disclosure herein.

1. A non-transitory computer-readable medium comprising executable instructions, the executable instructions being executable by one or more processors to perform a method, the method comprising:
receiving a first set of values, the first set of values based on a set of intensity measurements for a set of wavelengths of light, the set of intensity measurements for the set of wavelengths of light obtained by an apparatus configured to generate light, detect the light that has passed through at least a portion of a sample of a food processing byproduct, and measure intensities of the light to obtain the set of intensity measurements for the set of wavelengths of light;
generating a second set of values based on the first set of values;
applying a set of trained decision trees to the second set of values to obtain a result, the set of trained decision trees trained on a set of training samples, a first subset of training samples of the set of training samples containing a foodborne pathogen at a first concentration and a second subset of training samples of the set of training samples containing the foodborne pathogen at a second concentration different from the first concentration;
based on the result, determining either a positive foodborne pathogen detection or a negative foodborne pathogen detection for the foodborne pathogen in the sample of the food processing byproduct;
generating a foodborne pathogen detection notification that indicates either the positive foodborne pathogen detection or the negative foodborne pathogen detection for the foodborne pathogen in the sample of the food processing byproduct; and
providing the foodborne pathogen detection notification.

2. The non-transitory computer-readable medium of claim 1, the method further comprising based on the result, determining an approximate concentration or an approximate range of concentrations for the foodborne pathogen in the sample of the food processing byproduct, and wherein the foodborne pathogen detection notification further indicates the approximate concentration or the approximate range of concentrations for the foodborne pathogen in the sample of the food processing byproduct.

3. The non-transitory computer-readable medium of claim 1 wherein applying the set of trained decision trees to the second set of values to obtain the result further obtains a confidence value for the foodborne pathogen in the sample of the food processing byproduct, and wherein the foodborne pathogen detection notification further indicates the confidence value for the foodborne pathogen in the sample of the food processing byproduct.

4. The non-transitory computer-readable medium of claim 1 wherein the set of trained decision trees is a first set of trained decision trees, the set of training samples is a first set of training samples, the foodborne pathogen is a first foodborne pathogen, the result is a first result, the positive foodborne pathogen detection is a first positive foodborne pathogen detection, the negative foodborne pathogen detection is a first negative foodborne pathogen detection, and the foodborne pathogen detection notification is a first foodborne pathogen detection notification, the method further comprising:
applying a second set of trained decision trees to the second set of values to obtain a second result, the second set of trained decision trees trained on a second set of training samples, a third subset of training samples of the second set of training samples containing a second foodborne pathogen at a third concentration and a fourth subset of training samples of the second set of training samples containing the second foodborne pathogen at a fourth concentration different from the third concentration, the second foodborne pathogen different from the first foodborne pathogen;
based on the second result, determining either a second positive foodborne pathogen detection or a second negative foodborne pathogen detection for the second foodborne pathogen in the sample of the food processing byproduct;
generating a second foodborne pathogen detection notification indicating either the second positive foodborne pathogen detection or the second negative foodborne pathogen detection for the second foodborne pathogen in the sample of the food processing byproduct; and
providing the second foodborne pathogen detection notification.

5. The non-transitory computer-readable medium of claim 1 wherein the set of intensity measurements for the set of wavelengths of light is a first set of intensity measurements for the set of wavelengths of light, the result is a first result, the method further comprising:
receiving at least one third set of values, the at least one third set of values based on at least one second set of intensity measurements for the set of wavelengths of light, the at least one second set of intensity measurements for the set of wavelengths of light obtained by the apparatus;
generating at least one fourth set of values based on the at least one third set of values; and
applying the set of trained decision trees to the at least one fourth set of values to obtain at least one second result,
wherein based on the first result, determining either the positive foodborne pathogen detection or the negative foodborne pathogen detection for the foodborne pathogen in the sample of the food processing byproduct includes based on the first result and the at least one second result, determining either the positive foodborne pathogen detection or the negative foodborne pathogen detection for the foodborne pathogen in the sample of the food processing byproduct.

6. The non-transitory computer-readable medium of claim 1 wherein generating the second set of values based on the first set of values includes normalizing each value in the second set of values to be between zero, inclusive, and one, inclusive.

7. The non-transitory computer-readable medium of claim 1, the method further comprising training a set of decision trees on the set of training samples to obtain the set of trained decision trees.

8. The non-transitory computer-readable medium of claim 1 wherein at least some training samples of the set of training samples correspond to a particular food processing facility, a region that includes multiple food processing facilities, or one or more classes of food processing facilities.

9. The non-transitory computer-readable medium of claim 1 wherein values in the first set of values are one of absorbance values and transmittance values.

10. The non-transitory computer-readable medium of claim 1 wherein the sample of the food processing byproduct is mixed with a reagent.

11. The non-transitory computer-readable medium of claim 1 wherein the result indicates the positive foodborne pathogen detection if the result meets or exceeds a threshold.

12. The non-transitory computer-readable medium of claim 1 wherein the set of wavelengths of light includes wavelengths of light in ultraviolet, visible, and infrared spectrums.

13. The non-transitory computer-readable medium of claim 1 wherein the set of wavelengths of light includes wavelengths of light ranging from approximately 300 nanometers to approximately 1100 nanometers.

14. A system comprising at least one processor and memory containing executable instructions, the executable instructions being executable by the at least one processor to:
receive a first set of values, the first set of values based on a set of intensity measurements for a set of wavelengths of light, the set of intensity measurements for the set of wavelengths of light obtained by an apparatus configured to generate light, detect the light that has passed through at least a portion of a sample of a food processing byproduct, and measure intensities of the light to obtain the set of intensity measurements for the set of wavelengths of light;
generate a second set of values based on the first set of values;
apply a set of trained decision trees to the second set of values to obtain a result, the set of trained decision trees trained on a set of training samples, a first subset of training samples containing a foodborne pathogen at a first concentration and a second subset of training samples containing the foodborne pathogen at a second concentration different from the first concentration;
based on the result, determine either a positive foodborne pathogen detection or a negative foodborne pathogen detection for the foodborne pathogen in the sample of the food processing byproduct;
generate a foodborne pathogen detection notification that indicates either the positive foodborne pathogen detection or the negative foodborne pathogen detection for the foodborne pathogen in the sample of the food processing byproduct; and
provide the foodborne pathogen detection notification.

15. The system of claim 14, the executable instructions being further executable by the at least one processor to based on the result, determine an approximate concentration or an approximate range of concentrations for the foodborne pathogen in the sample of the food processing byproduct, and wherein the foodborne pathogen detection notification further indicates the approximate concentration or the approximate range of concentrations for the foodborne pathogen in the sample of the food processing byproduct.

16. The system of claim 14 wherein the executable instructions to apply the set of trained decision trees to the second set of values to obtain the result include executable instructions to obtains a confidence value for the foodborne pathogen in the sample of the food processing byproduct, and wherein the foodborne pathogen detection notification further indicates the confidence value for the foodborne pathogen in the sample of the food processing byproduct.

17. The system of claim 14 wherein the set of trained decision trees is a first set of trained decision trees, the set of training samples is a first set of training samples, the foodborne pathogen is a first foodborne pathogen, the result is a first result, the positive foodborne pathogen detection is a first positive foodborne pathogen detection, the negative foodborne pathogen detection is a first negative foodborne pathogen detection, and the foodborne pathogen detection notification is a first foodborne pathogen detection notification, and the executable instructions being further executable by the at least one processor to:
apply a second set of trained decision trees to the second set of values to obtain a second result, the second set of trained decision trees trained on a second set of training samples, a third subset of training samples containing a second foodborne pathogen at a third concentration and a fourth subset of training samples containing the second foodborne pathogen at a fourth concentration different from the third concentration, the second foodborne pathogen different from the first foodborne pathogen;
based on the second result, determine either a second positive foodborne pathogen detection or a second negative foodborne pathogen detection for the second foodborne pathogen in the sample of the food processing byproduct;
generate a second foodborne pathogen detection notification indicating either the second positive foodborne pathogen detection or the second negative foodborne pathogen detection for the second foodborne pathogen in the sample of the food processing byproduct; and
provide the second foodborne pathogen detection notification.

18. The system of claim 14 wherein the executable instructions being executable by the at least one processor to generate the second set of values based on the first set of values include executable instructions being executable by the at least one processor to normalize each value in the second set of values to be between zero, inclusive, and one, inclusive.

19. The system of claim 14, the executable instructions being further executable by the at least one processor to train a set of decision trees on the set of training samples to obtain the set of trained decision trees.

20. The system of claim 14 wherein at least some training samples of the set of training samples correspond to a particular food processing facility, a region that includes multiple food processing facilities, or one or more classes of food processing facilities.

21. The system of claim 14 wherein values in the first set of values are one of absorbance values and transmittance values.

22. The system of claim 14 wherein the sample of the food processing byproduct is mixed with a reagent.

23. The system of claim 14 wherein the result indicates the positive foodborne pathogen detection if the result meets or exceeds a threshold.

24. The system of claim 14 wherein the set of wavelengths of light includes wavelengths of light in ultraviolet, visible, and infrared spectrums.

25. A method comprising:
receiving a first set of values, the first set of values based on a set of intensity measurements for a set of wavelengths of light, the set of intensity measurements for the set of wavelengths of light obtained by an apparatus configured to generate light, detect the light that has passed through at least a portion of a sample of a food processing byproduct, and measure intensities of the light to obtain the set of intensity measurements for the set of wavelengths of light;
generating a second set of values based on the first set of values;
applying a set of trained decision trees to the second set of values to obtain a result, the set of trained decision trees trained on a set of training samples, a first subset of training samples containing a foodborne pathogen at a first concentration and a second subset of training samples containing the foodborne pathogen at a second concentration different from the first concentration;
based on the result, determining either a positive foodborne pathogen detection or a negative foodborne pathogen detection for the foodborne pathogen in the sample of the food processing byproduct;
generating a foodborne pathogen detection notification indicating either the positive foodborne pathogen detection or the negative foodborne pathogen detection for the foodborne pathogen in the sample of the food processing byproduct; and
providing the foodborne pathogen detection notification.
